SEQUENCE LISTING (1 sequence)

SEQ ID NO: 1 - protein, 25 aa, Artificial
  Synthetic
  MOD_RES (25)..(25): Biotinylation
SGRGKGGKGLGKGGAKRHRKVLRDK

(19) United States
(12) Patent Application Publication
(10) Pub. No.: US 2020/0123147 A1
(43) Pub. Date: Apr. 23, 2020

(54) THIAZOLE COMPOUNDS USEFUL AS PRMT5 INHIBITORS

(71) Applicants: Bayer Aktiengesellschaft, Leverkusen (DE); Bayer Pharma Aktiengesellschaft, Berlin (DE)

(72) Inventors: Lea Aurelie BOUCHE, Berlin (DE); Duy NGUYEN, Berlin (DE); Daniel KORR, Berlin (DE); Marcus KOPPITZ, Berlin (DE); Antonius TER LAAK, Berlin (DE); Amaury Ernesto FERNANDEZ-MONTALVAN, Le Pecq (FR); Thi Thanh Uyen NGUYEN, Berlin (DE); Stefan PRECHTL, Berlin (DE); Andreas JANZER, Berlin (DE)

(21) Appl. No.: 16/623,935

(22) PCT Filed: Jun. 21, 2018
(86) PCT No.: PCT/EP2018/066538

§ 371 Date: Dec. 18, 2019

(30) Foreign Application Priority Data
Jun. 29, 2017 (EP) 17178851.6

(51) Int. Cl. C07D 417/14; C07D 417/12; C07D 277/56; A61P 35/00

(52) U.S. Cl. CPC C07D 417/14; C07D 417/12; A61K 45/06; A61P 35/00; C07D 277/56

(57) ABSTRACT

The present invention provides PRMT5 inhibiting compounds of general formula (I): A compound of general formula (I),

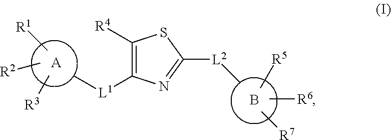

in which R¹, R², R³ R⁴, R⁵, R⁶, R⁷ L¹ and L² are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of disorders, in particular of hyperproliferative disorders, as a sole agent or in combination with other active ingredients.

The present invention provides thiazole compounds compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of disorders, in particular of hyperproliferative disorders, more particularly cancer disorders, as a sole agent or in combination with other active ingredients.

### BACKGROUND

The present invention provides Thiazole Compounds compounds of general formula (I) which inhibit PRMT5.

The PRMT family is comprised of eleven members and all PRMT members share common characteristics such as a highly conserved methyltransferase domain and the use of S-adenosyl-I-methionine (SAM) as a methyl donor. PRMTs catalyse methylation of guanidine nitrogen atoms of arginine residues leading to three different products: monomethylarginine (MMA), symmetric dimethylarginine (SDMA), and asymmetric dimethylarginine (ADMA). According to their substrate specificity they can be grouped into Type I (ADMA) and Type II (SDMA) PRMTs [Peng, C. and C. C. L. Wong (2017). Expert Review of Proteomics 14(2): 157-170.].

PRMT5, also named HRMT1L5, IBP72, JBP1, SKB1 or SKB1Hs, is the primary enzyme responsible for mono- and symmetric methylation in a cell and is conserved in all eukaryotic species investigated. Other Type II enzymes are PRMT7, PRMT9/FBXO11, PRMT10, and PRMT11/FBXO10. A unique feature of PRMT5 is the complex formation with the methylosome protein 50 (MEP50) that is essential for enzymatic activity [Stopa, N., et al. (2015). Cellular and Molecular Life Sciences 72(11): 2041-2059.]. The crystal structure of the human hetero-octameric complex (PRMT5)₄(MEP50)₄ co-crystallised with SAH and a histone H4 peptide was published and illustrates substrate binding and the mechanism of methylation [Antonysamy, S., et al. (2012). Proceedings of the National Academy of Sciences 109(44): 17960-17965.].

Histone tail modifications are major components of the epigenetic regulation of gene transcription. Arginine methylation of histones by PRMT5 occur at H2A and H4 on Arg3 and histone H3 on Arg2 and Arg8 and induces gene silencing at the respective promoters of PRMT5 target genes [Majumder, Sarmila et al. (2010). Journal of cellular biochemistry 109.3 (2010): 553-563; Burgos, E. S., et al. (2015). Journal of Biological Chemistry 290(15): 9674-9689.]. Known PRMT5 target genes include cyclin E1, Retinoblastoma protein and ribosomal genes [Fabbrizio, E., et al. (2002). EMBO reports 3(7): 641-645; Wang et al., Mol. Cell. Biol. October 2008 vol. 28 no. 20 6262-6277]. PRMT5 also regulates transcription through methylation of transcription factors, like p53, NFkB and E2F1 [Cho, E. C., et al. (2012). The EMBO Journal 31(7): 1785-1797; Jansson, M., et al. (2008). Nat Cell Biol 10(12): 1431-1439; Wei, H., et al. (2013). Proceedings of the National Academy of Sciences 110(33): 13516-13521.] Another major role of PRMT5 is the regulation of the spiceosome by methylation of the spiling factors SmD1, SmD3 and SmB/B′ on their C-termini. Methylation increases binding of these factors to Tudor domain-containing protein SMN (survival of motor neuron). SMN has in complex with other proteins a chaperone function for Sm proteins which is necessary for correct splicing [Meister, G., et al. (2001). Current Biology 11(24): 1990-1994.]. PRMT5 absence leads to selective retention of introns and skipping of exons with weak 5′ donor sites [Bezzi, Marco et al. Genes & Development 27.17 (2013): 1903-1916.]. Other PRMT5 substrates include ribosomal protein RPS10 (influencing ribosomal assembly), FEN1 (influencing DNA replication and repair), EGFR (reduces autophosphorylation) and Nucleoplasmin and Nucleolin (methylation effect unknown) [Ren, Jinqi et al. The Journal of Biological Chemistry 285.17 (2010): 12695-12705; Guo, Shaoshi, and Shilai Bao. The Journal of Biological Chemistry 285.45 (2010): 35133-35141; Hsu, Jung-Mao et al. Nature cell biology 13.2 (2011): 174-181.].

Increased expression of PRMT5 is found in prostate, ovarian, lung, lymphoid, lymphoma, glioblastoma multiforme, melanoma, colon, gastric, bladder cancer and germ cell tumors. Elevated PRMT5 expression in epithelial melanoma, ovarian and non-small cell lung cancer correlates with a poor prognosis [Stopa, N., et al. (2015). Cellular and Molecular Life Sciences 72(11): 2041-2059.]. Overexpression of PRMT5 results in increased proliferation and anchorage-independent growth. In nude mice overexpression of PRMT5 leads to tumor. On the opposite, PRMT5 knockdown inhibits cellular proliferation and colony formation in melanoma, lung and breast cancer cell lines [Wei, T.-Y. W., et al. (2012). Cancer Science 103(9): 1640-1650; Pal et al. (2004) Mol. Cell. Biol. November vol. 24 no. 21 9630-9645; Scoumanne, A., et al. (2009). Nucleic Acids Research 37(15): 4965-4976.] Therefore, PRMT5 is a valid target for the identification of novel cancer therapeutics.

Potential PRMT5 inhibitors are known in the literature [Hu, H., et al. (2016). Expert Opinion on Investigational Drugs 25(3): 335-358.]

From WO2011077133 PRMT5 modulators for the treatment of a proliferative disorder are known.

However, the state of the art does not describe the thiazole compounds of general formula (I) of the present invention as described and defined herein and up to now no clinical candidates are known so that it seems to be more than reasonable to provide further PRMT5 inhibitors for the treatment of hyperproliferative disorders.

In particular, the compounds of the present invention have surprisingly been found to effectively inhibit PRMT5 for which data are given in biological experimental section and may therefore for the reason as outlined above be used for the treatment or prophylaxis of hyperproliferative disorders, more particular cancer disorders, for example.

### DESCRIPTION OF THE INVENTION

In accordance with a first aspect, the present invention provides compounds of general formula (I):

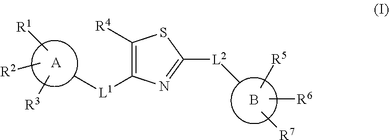

in which:

R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted with a halogen atom, a C₁-C₄-alkoxy group, a C₁-C₄-alkyl group;

R² is selected from a hydrogen atom, a C₁-C₆-alkyl group, a —O—(CH₂)₂—NR⁸R⁹ group and a cyano group;

R³ is selected from a hydrogen atom, a cyano group, a NR⁸R⁹ group, a C₁-C₆-alkyl group, a C₁-C₆-hydroxyalkyl group and a heterocycloalkyl group;

R⁴ is hydrogen;

R⁵ is selected from a hydrogen atom, a halogen atom, a cyano group, a C₁-C₆-alkyl group and a N(O)₂ group;

R⁶ is selected from a hydrogen atom, a halogen atom, a C₁-C₆-alkyl group, a C₂-C₆-alkenyl group and a C₂-C₆-alkynyl group;

R⁷ is selected from a hydrogen atom, a halogen atom and a C₁-C₆-alkyl group;

R⁸, R⁹ is independently selected from a hydrogen atom and a C₁-C₆-alkyl group;

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

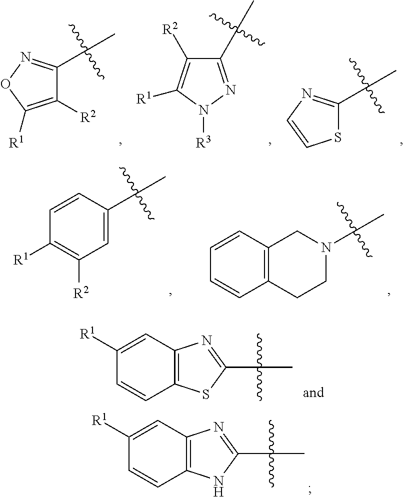

L¹ is selected from ^{$}—NH—C(O)—^{$$}, ^{$}—(CH₂)₃—NH—C(O)—^{$$}, ^{$}—(CH₂)₄—NH—C(O)—^{$$},

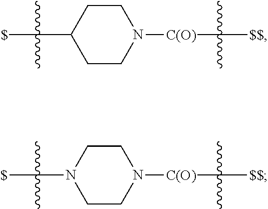

^{$} is the point of attachment of L¹ to ring A and ^{$$} is the point of attachment of L¹ to the thiazole carbon atom;

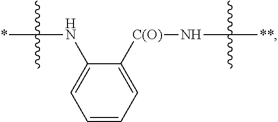

L² is selected from a bond, *—NH—**, *—NH—CH₂—**, *—NH—(CH₂)₂—**, *—CH₂—**; and * is the point of attachment of L² to the thiazole carbon atom and ** is the point of attachment of L² to ring B;

B is selected from

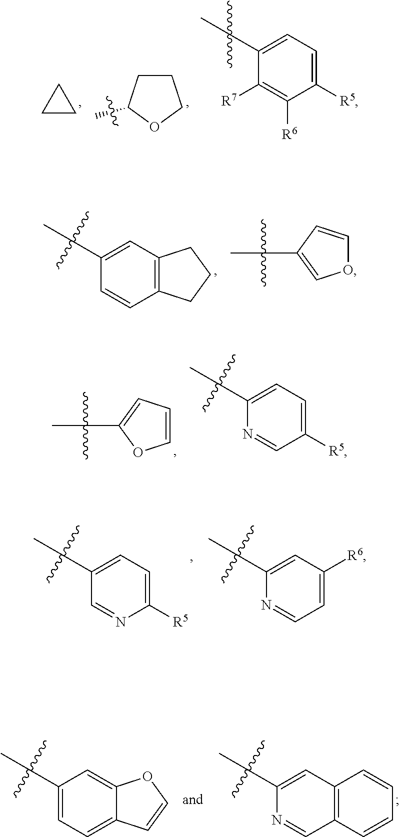

or a stereoisomer, a tautomer, a hydrate, a solvate, or a salt thereof, or a mixture of same.

In accordance with an embodiment of the first aspect, the present invention provides compounds of general formula (I) wherein

R¹ is selected from a hydrogen atom, a halogen atom, a C₁-C₄-alkyl group, a C₁-C₄-haloalkyl group, a C₃-C₆-cycloalkyl group, a C₁-C₄-alkoxy group and a phenyl group which is optionally substituted with a halogen atom, a C₁-C₃-alkoxy group, a C₁-C₃-alkyl group;

R² is selected from a hydrogen atom, a C₁-C₄-alkyl group, a —O—(CH₂)₂—NR⁸R⁹ group and a cyano group;

R³ is selected from a hydrogen atom, a cyano group, a NR⁸R⁹ group, a C₁-C₄-alkyl group, a C₁-C₄-hydroxyalkyl group and a heterocycloalkyl group;

R⁴ is hydrogen;

R⁵ is selected from a hydrogen atom, a halogen atom, a cyano group, a C₁-C₄-alkyl group and a N(O)₂ group;

R⁶ is selected from a hydrogen atom, a halogen atom, a C₁-C₄-alkyl group, a C₂-C₄-alkenyl group and a C₂-C₄-alkynyl group;

R⁷ is selected from a hydrogen atom, a halogen atom and a C₁-C₄-alkyl group;

R⁸, R⁹ is independently selected from a hydrogen atom and a C₁-C₄-alkyl group;

A is selected from

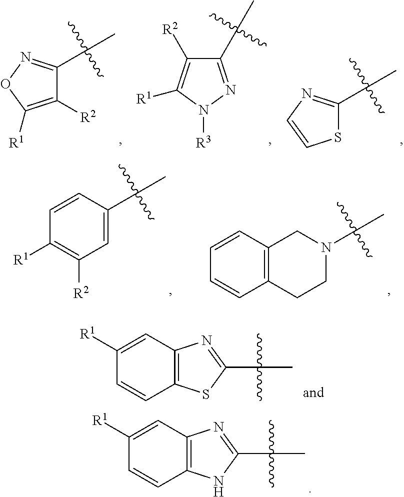

L¹ is selected from ^{$}—NH—C(O)—^{$$}, ^{$}—(CH₂)₃—NH—C(O)—^{$$}, ^{$}—(CH₂)₄—NH—C(O)—^{$$},

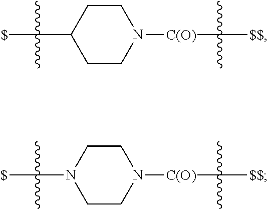

^{$} is the point of attachment of L¹ to ring A and ^{$$} is the point of attachment of L¹ to the thiazole carbon atom;

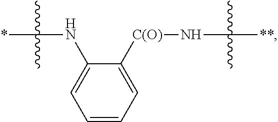

L² is selected from a bond, *—NH—**, *—NH—CH₂—**, *—NH—(CH₂)₂—**, *—CH₂—**; and * is the point of attachment of L² to the thiazole carbon atom and ** is the point of attachment of L² to ring B;

B is selected from

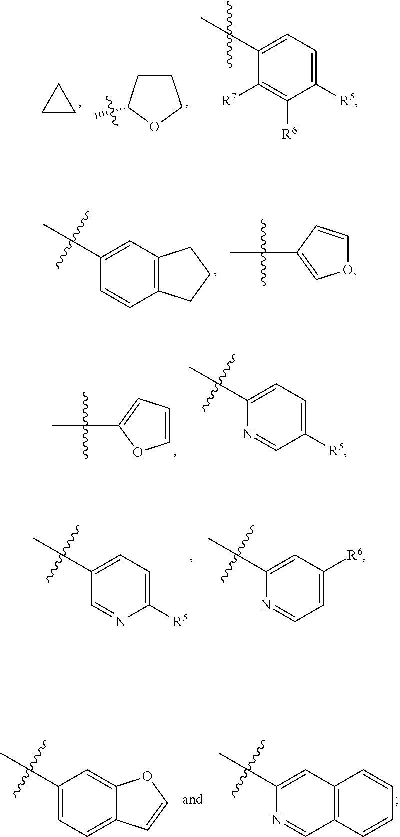

or a stereoisomer, a tautomer, a hydrate, a solvate, or a salt thereof, or a mixture of same.

In accordance with a further embodiment of the first aspect, the present invention provides compounds of general formula (I) wherein

R¹ is selected from a hydrogen atom, a C₁-C₄-alkyl group, a C₁-C₃-haloalkyl group, a C₃-C₆-cycloalkyl group, a C₁-C₃-alkoxy group and a phenyl group which is optionally substituted with a halogen atom, a C₁-C₃-alkoxy group, a C₁-C₃-alkyl group;

R² is selected from a hydrogen atom, a C₁-C₃-alkyl group, a —O—(CH₂)₂—NR⁸R⁹ group and a cyano group;

R³ is selected from a hydrogen atom, a cyano group, a NR⁸R⁹ group, a C₁-C₃-alkyl group, a C₁-C₃-hydroxyalkyl group and a heterocycloalkyl group;

R⁴ is hydrogen;

R⁵ is selected from a hydrogen atom, a fluorine atom, a chlorine atom, a cyano group, a C₁-C₃-alkyl group and a N(O)₂ group;

R⁶ is selected from a hydrogen atom, a fluorine atom, a chlorine atom, a C₁-C₃-alkyl group and a C₂-C₃-alkynyl group;

R⁷ is selected from a hydrogen atom, a fluorine atom and a C₁-C₃-alkyl group;

R⁸, R⁹ is independently selected from a hydrogen atom and a C₁-C₃-alkyl group;

A is selected from

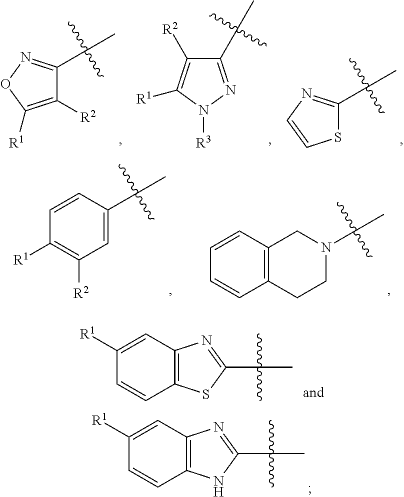

L¹ is selected from ^{$}—NH—C(O)—^{$$}, ^{$}—(CH₂)₃—NH—C(O)—^{$$}, ^{$}—(CH₂)₄—NH—C(O)—^{$$},

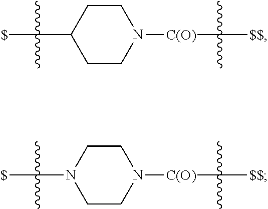

and ^{$} is the point of attachment of L¹ to ring A and ^{$$} is the point of attachment of L¹ to the thiazole carbon atom;

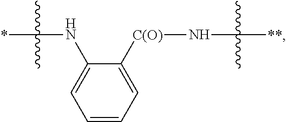

L² is selected from a bond, *—NH—**, *—NH—CH₂—**, *—NH—(CH₂)₂—**, *—CH₂—**; and * is the point of attachment of L² to the thiazole carbon atom and ** is the point of attachment of L² to ring B;

B is selected from

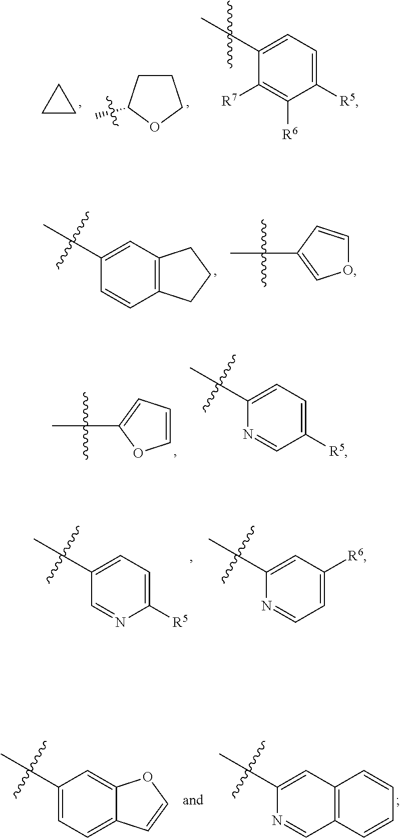

or a stereoisomer, a tautomer, a hydrate, a solvate, or a salt thereof, or a mixture of same.

In accordance with another embodiment of the a first aspect, the present invention provides compounds of general formula (I) wherein

R¹ is selected from a hydrogen atom, a C₁-C₄-alkyl group, a C₁-C₃-haloalkyl group, a C₃-C₆-cycloalkyl group, a C₁-C₄-alkoxy group and a phenyl group which is optionally substituted with a C₁-C₄-alkyl group;

R² is selected from a hydrogen atom, a C₁-C₃-alkyl group, a —O—(CH₂)₂—NR⁸R⁹ group and a cyano group;

R³ is selected from a hydrogen atom, a cyano group, a C₁-C₃-alkyl group, a heterocycloalkyl group and a C₁-C₃-hydroxyalkyl group;

R⁴ is hydrogen;

R⁵ is selected from a hydrogen atom, a fluorine atom, a chlorine atom, a cyano group, a C₁-C₃-alkyl group, and a N(O)₂ group,

R⁶ is selected from a hydrogen atom, a fluorine atom, a chlorine atom, a C₁-C₃-alkyl group, and a C₂-C₄-alkynyl group,

R⁷ is selected from a hydrogen atom, a fluorine atom, and a C₁-C₃-alkyl group,

R⁸, R⁹ is independently selected from a hydrogen atom and a C₁-C₃-alkyl group;

A is selected from

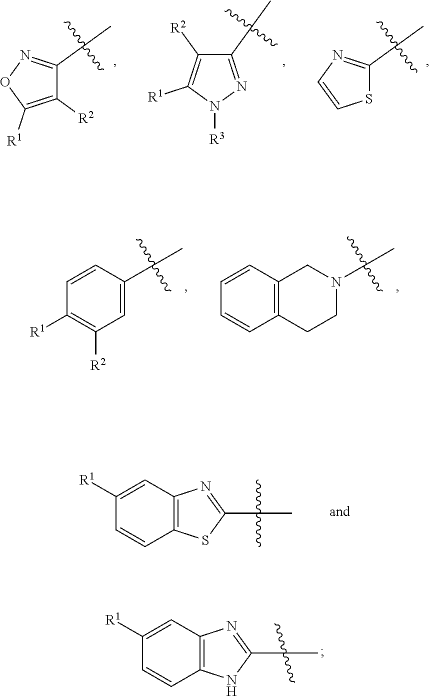

L¹ is selected from ^{$}—NH—C(O)—^{$$}, ^{$}—(CH₂)₃—NH—C(O)—^{$$}, ^{$}—(CH₂)₄—NH—C(O)—^{$$},

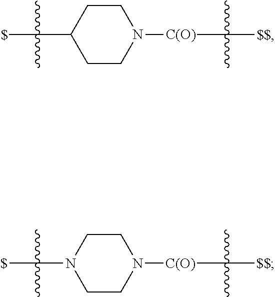

and ^{$} is the point of attachment of L¹ to ring A and ^{$$} is the point of attachment of L¹ to the thiazole carbon atom;

L² is selected from a bond, *—NH—**, *—NH—CH₂—**, *—NH—(CH₂)₂—**,

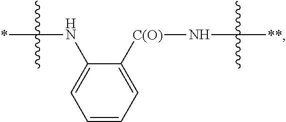

*—CH₂—**; and * is the point of attachment of L² to the thiazole carbon atom and ** is the point of attachment of L² to ring B;

B is selected from

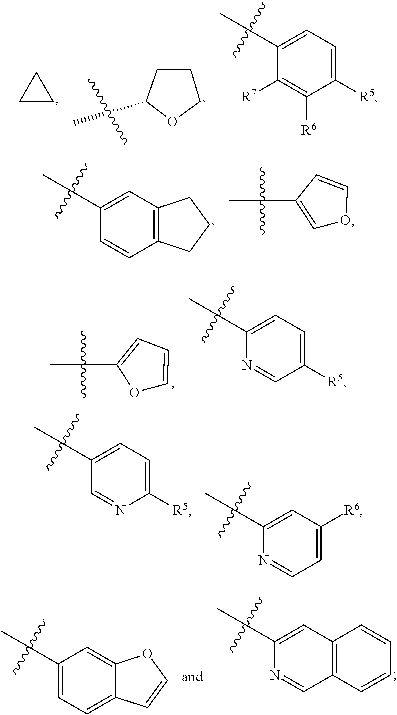

or a stereoisomer, a tautomer, a hydrate, a solvate, or a salt thereof, or a mixture of same.

In accordance with an embodiment of the a first aspect, the present invention provides compounds of general formula (I) wherein

R¹ is selected from a hydrogen atom, a methyl group, a tert-butyl group, a trifluoromethyl group, a cyclopropyl group, a methoxy group, a phenyl group and a 4-methylphenyl group;

R² is selected from a hydrogen atom, a methyl group, a —O—(CH₂)₂—N(CH₂—CH₃)₂ group and a cyano group;

R³ is selected from a hydrogen atom, a cyano group, a methyl group, a oxetan-3-yl-group and a hydroxyethyl group;

R⁴ is hydrogen;

R⁵ is selected from a hydrogen atom, a fluorine atom, a chlorine atom, a cyano group, a methyl group and a N(O)₂ group,

R⁶ is selected from a hydrogen atom, a fluorine atom, a chlorine atom, a methyl group and a ethinyl group,

R⁷ is selected from a hydrogen atom, a fluorine atom, and a methyl group,

R⁸, R⁹ is independently selected from a hydrogen atom and a methyl group;

A is selected from

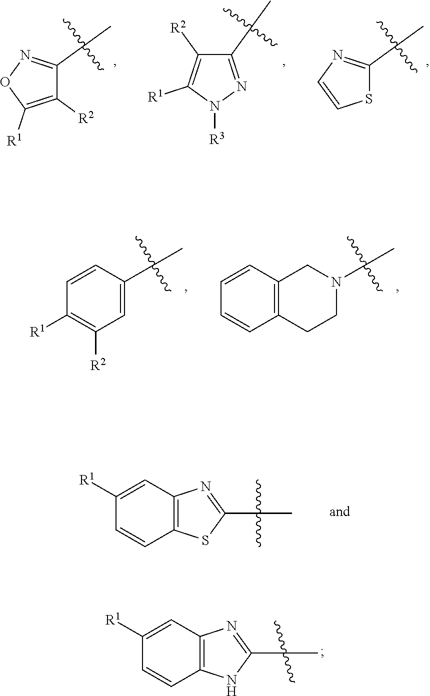

L¹ is selected from ^{$}—NH—C(O)—^{$$}, ^{$}—(CH₂)₃—NH—C(O)—^{$$}, ^{$}—(CH₂)₄—NH—C(O)—^{$$},

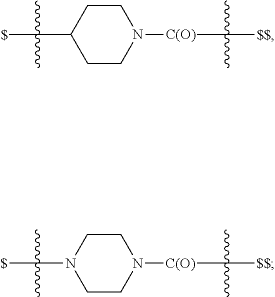

and ^{$} is the point of attachment of L¹ to ring A and ^{$$} is the point of attachment of L¹ to the thiazole carbon atom;

L² is selected from a bond, *—NH—**, *—NH—CH₂—**, *—NH—(CH₂)₂—**,

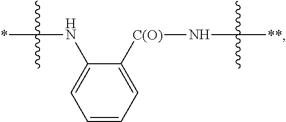

*—CH₂—**; and * is the point of attachment of L² to the thiazole carbon atom and ** is the point of attachment of L² to ring B;

B is selected from

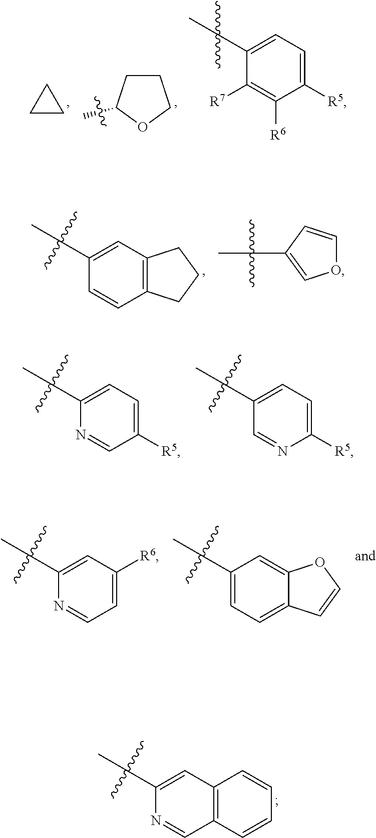

or a stereoisomer, a tautomer, a hydrate, a solvate, or a salt thereof, or a mixture of same.

In accordance with a further aspect, the present invention provides compounds of general formula (I):

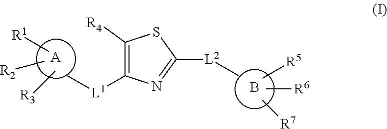

in which:

R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted with a halogen atom, a C₁-C₄-alkoxy group, a C₁-C₄-alkyl group;

R² is selected from a hydrogen atom, a C₁-C₆-alkyl group, a —O—(CH₂)₂—NR⁸R⁹ group and a cyano group;

R³ is selected from a hydrogen atom, a cyano group, a NR⁸R⁹ group, a C₁-C₆-alkyl group, a C₁-C₆-hydroxyalkyl group and a heterocycloalkyl group;

R⁴ is hydrogen;

R⁵ is selected from a hydrogen atom, a halogen atom, a cyano group, a C₁-C₆-alkyl group and a N(O)₂ group;

R⁶ is selected from a hydrogen atom, a halogen atom, a C₁-C₆-alkyl group, a C₂-C₆-alkenyl group and a C₂-C₆-alkynyl group;

R⁷ is selected from a hydrogen atom, a halogen atom and a C₁-C₆-alkyl group;

R⁸, R⁹ is independently selected from a hydrogen atom and a C₁-C₆-alkyl group;

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

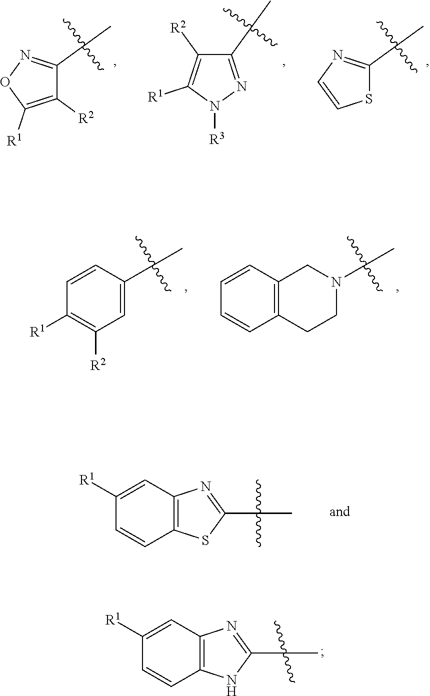

L¹ is selected from ^{$}—NH—C(O)—^{$$}, ^{$}—(CH₂)₃—NH—C(O)—^{$$}, ^{$}—(CH₂)₄—NH—C(O)—^{$$},

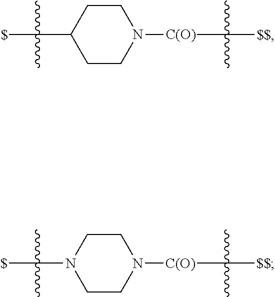

^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

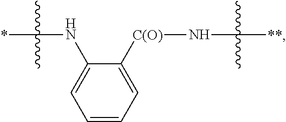

—CH₂—; and * is the point of attachment of L² to the thiazole carbon atom and ** is the point of attachment of L² to ring B;

B is selected from

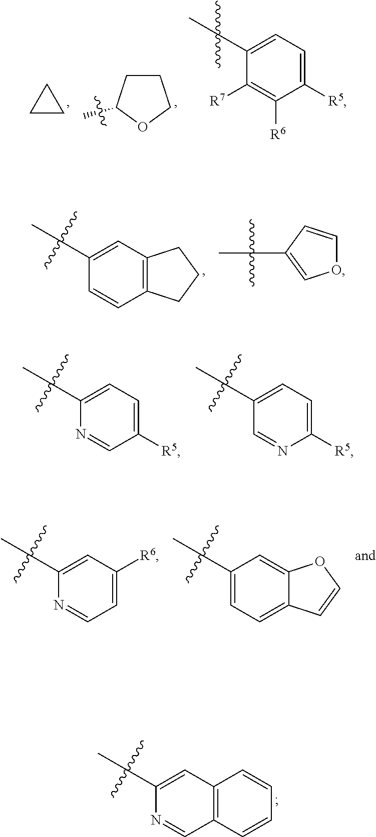

or a stereoisomer, a tautomer, a hydrate, a solvate, or a salt thereof, or a mixture of same.

In accordance with an embodiment of the first aspect, the present invention provides compounds of general formula (I) wherein

R¹ is selected from a hydrogen atom, a halogen atom, a C₁-C₄-alkyl group, a C₁-C₄-haloalkyl group, a C₃-C₆-cycloalkyl group, a C₁-C₄-alkoxy group and a phenyl group which is optionally substituted with a halogen atom, a C₁-C₃-alkoxy group, a C₁-C₃-alkyl group;

R² is selected from a hydrogen atom, a C₁-C₄-alkyl group, a —O—(CH₂)₂—NR⁸R⁹ group and a cyano group;

R³ is selected from a hydrogen atom, a cyano group, a NR⁸R⁹ group, a C₁-C₄-alkyl group, a C₁-C₄-hydroxyalkyl group and a heterocycloalkyl group;

R⁴ is hydrogen;

R⁵ is selected from a hydrogen atom, a halogen atom, a cyano group, a C₁-C₄-alkyl group and a N(O)₂ group;

R⁶ is selected from a hydrogen atom, a halogen atom, a C₁-C₄-alkyl group, a C₂-C₄-alkenyl group and a C₂-C₄-alkynyl group;

R⁷ is selected from a hydrogen atom, a halogen atom and a C₁-C₄-alkyl group;

R⁸, R⁹ is independently selected from a hydrogen atom and a C₁-C₄-alkyl group;

A is selected from

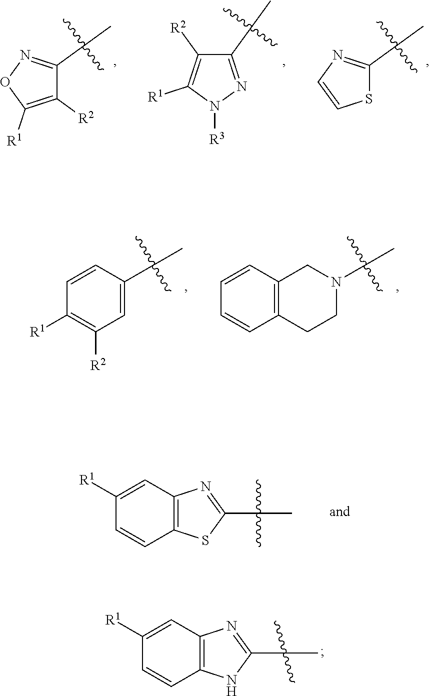

L¹ is selected from ^{$}—NH—C(O)—^{$$}, ^{$}—(CH₂)₃—NH—C(O)—^{$$}, ^{$}—(CH₂)₄—NH—C(O)—^{$$},

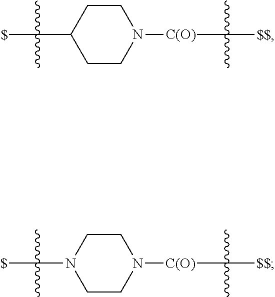

^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

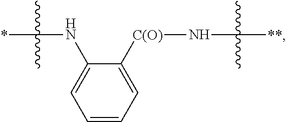

, —CH₂—; and * is the point of attachment of L² to the thiazole carbon atom and ** is the point of attachment of L² to ring B;

B is selected from

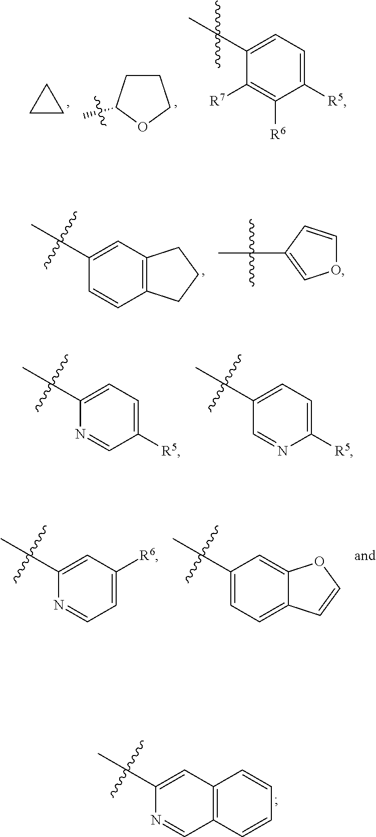

or a stereoisomer, a tautomer, a hydrate, a solvate, or a salt thereof, or a mixture of same.

In accordance with a further embodiment of the first aspect, the present invention provides compounds of general formula (I) wherein

R¹ is selected from a hydrogen atom, a C₁-C₄-alkyl group, a C₁-C₃-haloalkyl group, a C₃-C₆-cycloalkyl group, a C₁-C₃-alkoxy group and a phenyl group which is optionally substituted with a halogen atom, a C₁-C₃-alkoxy group, a C₁-C₃-alkyl group;

R² is selected from a hydrogen atom, a C₁-C₃-alkyl group, a —O—(CH₂)₂—NR⁸R⁹ group and a cyano group;

R³ is selected from a hydrogen atom, a cyano group, a NR⁸R⁹ group, a C₁-C₃-alkyl group, a C₁-C₃-hydroxyalkyl group and a heterocycloalkyl group;

R⁴ is hydrogen;

R⁵ is selected from a hydrogen atom, a fluorine atom, a chlorine atom, a cyano group, a C₁-C₃-alkyl group and a N(O)₂ group;

R⁶ is selected from a hydrogen atom, a fluorine atom, a chlorine atom, a C₁-C₃-alkyl group and a C₂-C₃-alkynyl group;

R⁷ is selected from a hydrogen atom, a fluorine atom and a C₁-C₃-alkyl group;

R⁸, R⁹ is independently selected from a hydrogen atom and a C₁-C₃-alkyl group;

A is selected from

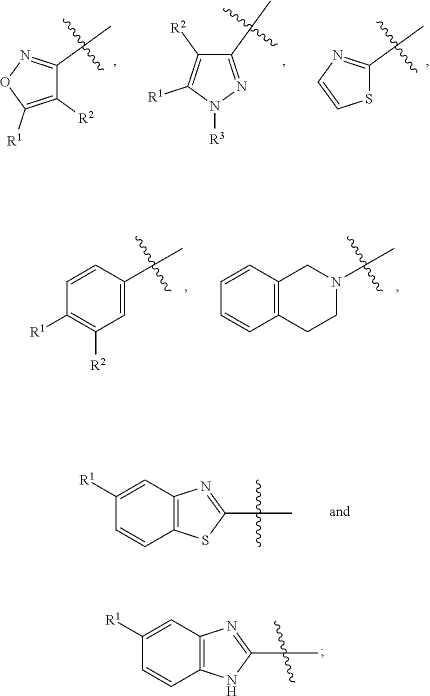

L¹ is selected from ^{$}—NH—C(O)—^{$$}, ^{$}—(CH₂)₃—NH—C(O)—^{$$}, ^{$}—(CH₂)₄—NH—C(O)—^{$$},

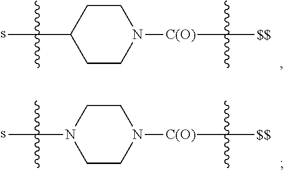

and ^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

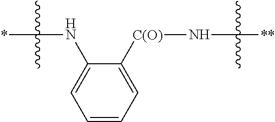

, —CH₂—; and * is the point of attachment of L² to the thiazole carbon atom and ** is the point of attachment of L² to ring B;

B is selected from

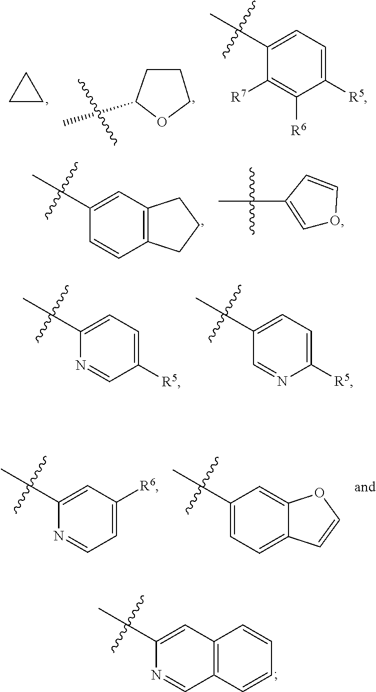

or a stereoisomer, a tautomer, a hydrate, a solvate, or a salt thereof, or a mixture of same.

In accordance with an embodiment of the a first aspect, the present invention provides compounds of general formula (I) wherein

R¹ is selected from a hydrogen atom, a methyl group, a tert-butyl group, a trifluoromethyl group, a cyclopropyl group, a methoxy group, a phenyl group and a 4-methylphenyl group;

R² is selected from a hydrogen atom, a methyl group, a —O—(CH₂)₂—N(CH₂—CH₃)₂ group and a cyano group;

R³ is selected from a hydrogen atom, a cyano group, a methyl group, a oxetan-3-yl-group and a hydroxyethyl group;

R⁴ is hydrogen;

R⁵ is selected from a hydrogen atom, a fluorine atom, a chlorine atom, a cyano group, a methyl group and a N(O)₂ group,

R⁶ is selected from a hydrogen atom, a fluorine atom, a chlorine atom, a methyl group and a ethinyl group,

R⁷ is selected from a hydrogen atom, a fluorine atom, and a methyl group,

R⁸, R⁹ is independently selected from a hydrogen atom and a methyl group;

A is selected from

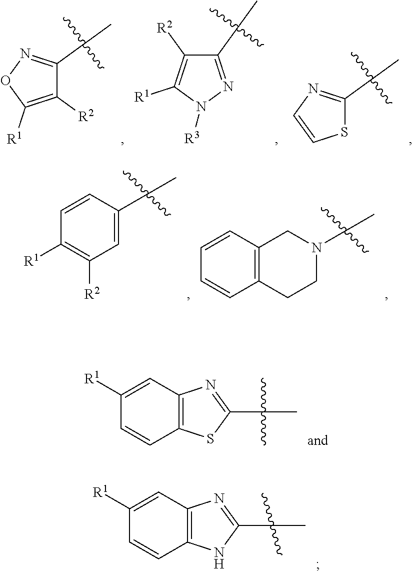

L¹ is selected from ^{$}—NH—C(O)—^{$$}, ^{$}—(CH₂)₃—NH—C(O)—^{$$}, ^{$}—(CH₂)₄—NH—C(O)—^{$$},

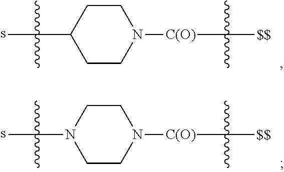

and ^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

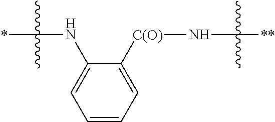

, —CH₂—; and * is the point of attachment of L² to the thiazole carbon atom and ** is the point of attachment of L² to ring B;

B is selected from

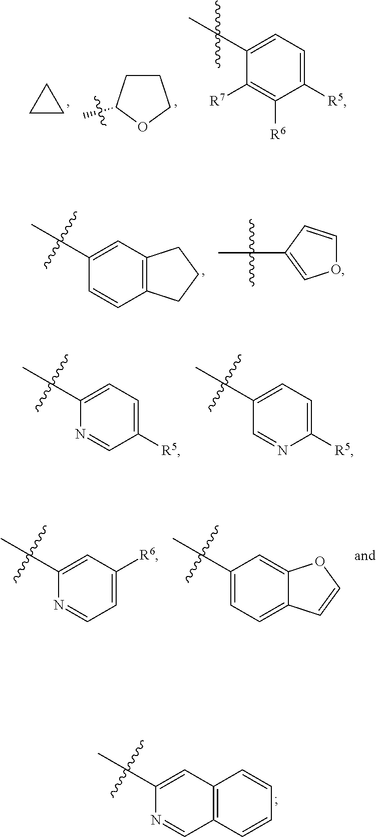

or a stereoisomer, a tautomer, a hydrate, a solvate, or a salt thereof, or a mixture of same.

In accordance with an embodiment of the a first aspect, the present invention provides compounds of general formula (I) wherein

R¹ is selected from a hydrogen atom, a methyl group, a tert-butyl group, a trifluoromethyl group, a cyclopropyl group, a methoxy group, a phenyl group and a 4-methylphenyl group;

R² is selected from a hydrogen atom, a methyl group, a —O—(CH₂)₂—N(CH₂—CH₃)₂ group and a cyano group;

R³ is selected from a hydrogen atom, a cyano group, a methyl group, a oxetan-3-yl-group and a hydroxyethyl group;

R⁴ is hydrogen;

R⁵ is selected from a hydrogen atom, a fluorine atom, a chlorine atom, a cyano group, a methyl group and a N(O)₂ group,

R⁶ is selected from a hydrogen atom, a fluorine atom, a chlorine atom, a methyl group and a ethinyl group,

R⁷ is selected from a hydrogen atom, a fluorine atom, and a methyl group,

R⁸, R⁹ is independently selected from a hydrogen atom and a methyl group;

A is selected from

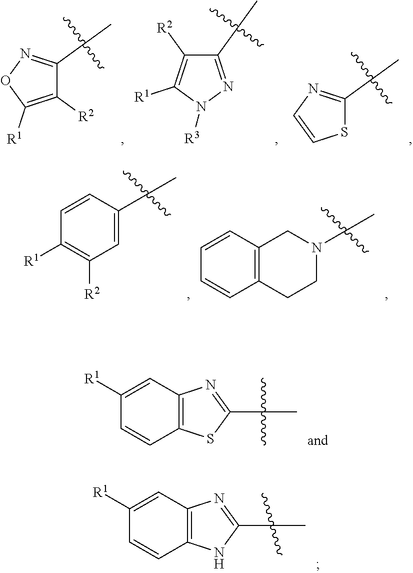

L¹ is selected from ^{$}—NH—C(O)—^{$$}, ^{$}—(CH₂)₃—NH—C(O)—^{$$}, ^{$}—(CH₂)₄—NH—C(O)—^{$$},

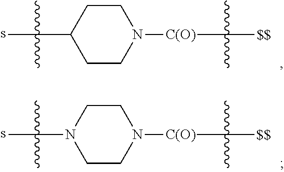

and ^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

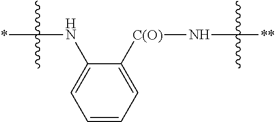

, —CH₂—; and * is the point of attachment of L² to the thiazole carbon atom and ** is the point of attachment of L² to ring B;

B is selected from

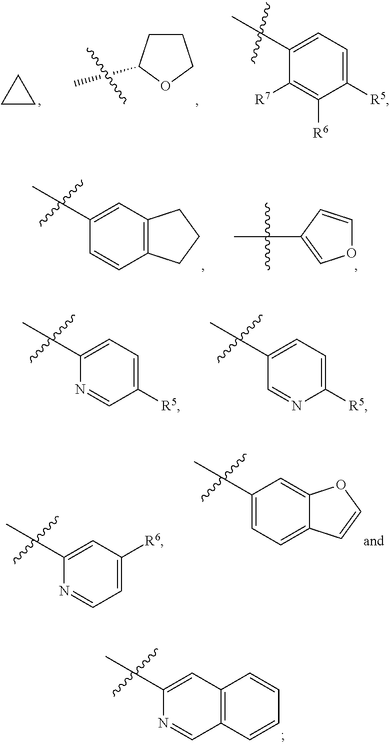

or a stereoisomer, a tautomer, a hydrate, a solvate, or a salt thereof, or a mixture of same.

In accordance with an embodiment of the a first aspect, the present invention provides compounds of general formula (I) selected from

2-[(4-Chlorophenyl)amino]-N-(5-phenyl-1H-pyrazol-3-yl)-1,3-thiazole-4-carboxamide,

N-(5-tert-Butyl-1,2-oxazol-3-yl)-2-(pyridin-2-ylamino)-1,3-thiazole-4-carboxamide,

N-(5-tert-Butyl-1,2-oxazol-3-yl)-2-(phenylamino)-1,3-thiazole-4-carboxamide,

N-(5-tert-Butyl-1,2-oxazol-3-yl)-2-[(4-chlorophenyl)amino]-1,3-thiazole-4-carboxamide,

2-[(4-Chlorophenyl)amino]-N-(3-cyclopropyl-1H-pyrazol-5-yl)-1,3-thiazole-4-carboxamide,

N-(5-tert-Butyl-1,2-oxazol-3-yl)-2-[(5-fluoropyridin-2-yl)amino]-1,3-thiazole-4-carboxamide,

N-(5-tert-Butyl-1,2-oxazol-3-yl)-2-[(4-fluorophenyl)amino]-1,3-thiazole-4-carboxamide,

N-(1,3-Benzothiazol-2-yl)-2-[(4-chlorophenyl)amino]-1,3-thiazole-4-carboxamide,

2-[(4-Chlorophenyl)amino]-N-(5-methoxy-1H-benzimidazol-2-yl)-1,3-thiazole-4-carboxamide,

N-[1-Methyl-5-(trifluoromethyl)-1H-pyrazol-3-yl]-2-(phenylamino)-1,3-thiazole-4-carboxamide,

2-[(4-Chlorophenyl)amino]-N-(1,3-thiazol-2-yl)-1,3-thiazole-4-carboxamide,

N-(5-tert-Butyl-1H-pyrazol-3-yl)-2-[(4-chlorophenyl)amino]-1,3-thiazole-4-carboxamide,

N-(5-tert-Butyl-1,2-oxazol-3-yl)-2-[(4-nitrophenyl)amino]-1,3-thiazole-4-carboxamide,

N-(3-Cyclopropyl-1H-pyrazol-5-yl)-2-(phenylamino)-1,3-thiazole-4-carboxamide,

2-[(4-Chlorophenyl)amino]-N-(1-ethyl-1H-pyrazol-3-yl)-1,3-thiazole-4-carboxamide,

N-(5-tert-Butyl-1H-pyrazol-3-yl)-2-(phenylamino)-1,3-thiazole-4-carboxamide,

2-[(4-Chlorophenyl)amino]-N[4-(3,4-dihydroisoquinolin-2(1H)-yl)butyl]-1,3-thiazole-4-carboxamide,

N-(5-tert-Butyl-1,2-oxazol-3-yl)-2-[(3-chloro-4-methylphenyl)amino]-1,3-thiazole-4-carboxamide,

2-[(4-Chlorophenyl)amino]-N-(1,2-oxazol-3-yl)-1,3-thiazole-4-carboxamide,

2-[(4-Chlorophenyl)amino]-N-(5-methyl-1,2-oxazol-3-yl)-1,3-thiazole-4-carboxamide,

N-(5-tert-Butyl-1,2-oxazol-3-yl)-2-[(3,4-dichlorophenyl)amino]-1,3-thiazole-4-carboxamide,

N-{2-[2-(Diethylamino)ethoxy]-4′-methylbiphenyl-4-yl}-2-(phenylamino)-1,3-thiazole-4-carboxamide,

N-(1-Methyl-1H-pyrazol-3-yl)-2-(phenylamino)-1,3-thiazole-4-carboxamide,

N-(5-tert-Butyl-1,2-oxazol-3-yl)-2-[(4-cyanophenyl)amino]-1,3-thiazole-4-carboxamide,

N-(5-tert-Butyl-1,2-oxazol-3-yl)-2-[(2-fluorophenyl)amino]-1,3-thiazole-4-carboxamide,

2-[(4-Chlorophenyl)amino]-N-[3-(3,4-dihydroisoquinolin-2(1H)-yl)propyl]-1,3-thiazole-4-carboxamide,

{2-[(4-Chlorophenyl)amino]-1,3-thiazol-4-yl}{4-[3,4-dihydroisoquinolin-2(1H)-yl]piperidin-1-yl}methanone,

N-[1-(2-Hydroxyethyl)-1H-pyrazol-3-yl]-2-(phenylamino)-1,3-thiazole-4-carboxamide,

N-(5-tert-Butyl-4-methyl-1,2-oxazol-3-yl)-2-[(4-chlorophenyl)amino]-1,3-thiazole-4-carboxamide,

N-(4-Cyano-1H-pyrazol-3-yl)-2-(phenylamino)-1,3-thiazole-4-carboxamide,

2-[(4-Chlorophenyl)amino]-N-[1-(oxetan-3-yl)-1H-pyrazol-4-yl]-1,3-thiazole-4-carboxamide,

N-(5-tert-Butyl-1,2-oxazol-3-yl)-2-(cyclopropylamino)-1,3-thiazole-4-carboxamide, and

{2-[(4-Chlorophenyl)amino]-1,3-thiazol-4-yl}[4-(5-methyl-1,3-benzothiazol-2-yl)piperazin-1-yl]methanone

N-(5-tert-Butyl-1,2-oxazol-3-yl)-2-[(5-methylpyridin-2-yl)amino]-1,3-thiazole-4-carboxamide,

N-(5-tert-Butyl-1,2-oxazol-3-yl)-2-[(5-chloropyridin-2-yl)amino]-1,3-thiazole-4-carboxamide,

N-(5-tert-Butyl-1,2-oxazol-3-yl)-2-[(4-chloropyridin-2-yl)amino]-1,3-thiazole-4-carboxamide,

N-(5-tert-Butyl-1,2-oxazol-3-yl)-2-[(2-phenylethyl)amino]-1,3-thiazole-4-carboxamide,

N-(5-tert-Butyl-1,2-oxazol-3-yl)-2-(2,3-dihydro-1H-inden-5-ylamino)-1,3-thiazole-4-carboxamide,

N-(5-tert-Butyl-1,2-oxazol-3-yl)-2-[(4-chloro-3-fluorophenyl)amino]-1,3-thiazole-4-carboxamide,

2-(1-Benzofuran-6-ylamino)-N-(5-tert-butyl-1,2-oxazol-3-yl)-1,3-thiazole-4-carboxamide,

N-(5-tert-Butyl-1,2-oxazol-3-yl)-2-[(2-fluoro-4-methylphenyl)amino]-1,3-thiazole-4-carboxamide,

N-(5-tert-Butyl-1,2-oxazol-3-yl)-2-[(3-fluorobenzyl)amino]-1,3-thiazole-4-carboxamide,

N-(5-tert-Butyl-1,2-oxazol-3-yl)-2-[(3-methylphenyl)amino]-1,3-thiazole-4-carboxamide,

N-(5-tert-Butyl-1,2-oxazol-3-yl)-2-[(furan-2-ylmethyl)amino]-1,3-thiazole-4-carboxamide,

N-(5-tert-Butyl-1,2-oxazol-3-yl)-2-[(3,4-difluorophenyl)amino]-1,3-thiazole-4-carboxamide,

N-(5-tert-Butyl-1,2-oxazol-3-yl)-2-[(3-chloro-4-fluorophenyl)amino]-1,3-thiazole-4-carboxamide,

N-(5-tert-Butyl-1,2-oxazol-3-yl)-2-{[2-(isoquinolin-3-ylcarbamoyl)phenyl]amino}-1,3-thiazole-4-carboxamide,

N-(5-tert-Butyl-1,2-oxazol-3-yl)-2-[(3-chlorophenyl)amino]-1,3-thiazole-4-carboxamide,

(rac)-N-(5-tert-Butyl-1,2-oxazol-3-yl)-2-{[-tetrahydrofuran-2-ylmethyl]amino}-1,3-thiazole-4-carboxamide,

N-(5-tert-Butyl-1,2-oxazol-3-yl)-2-[(4-fluoro-2-methylphenyl)amino]-1,3-thiazole-4-carboxamide,

N-(5-tert-Butyl-1,2-oxazol-3-yl)-2-[(3-ethynylphenyl)amino]-1,3-thiazole-4-carboxamide,

N-(5-tert-Butyl-1,2-oxazol-3-yl)-2-[(2,3-difluorophenyl)amino]-1,3-thiazole-4-carboxamide and

N-(5-tert-Butyl-1,2-oxazol-3-yl)-2-[(6-fluoropyridin-3-yl)amino]-1,3-thiazole-4-carboxamide

or a stereoisomer, a tautomer, a hydrate, a solvate, or a salt thereof, or a mixture of same.

Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, or 3, in particular 1, or 2.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom, more particularly a chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, or 2 hydrogen atoms, more particularly 1 hydrogen atom, are/is replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₂-C₆-alkenyl” means a linear or branched, monovalent hydrocarbon group, which contains one double bond, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl, more particularly said alkynyl group is ethynyl.

The term “C₃-C₆-cycloalkyl” means a saturated, monovalent, mono- or bicyclic hydrocarbon ring which contains 3, 4, 5, or 6 carbon atoms (“C₃-C₆-cycloalkyl”). Said C₃-C₆-cycloalkyl group is for example, a monocyclic hydrocarbon ring, e.g. a cyclopropyl, cyclobutyl, cyclopentyl or cyclohexyl group, or a bicyclic hydrocarbon ring, e.g. a bicyclo[4.2.0]octyl or octahydropentalenyl. Particularly said cycloalkyl group is cyclopropyl.

The term “heterocycloalkyl” includes “4- to 7-membered heterocycloalkyl”, “4- to 6-membered heterocycloalkyl” and “fused heterocycloalkyl”. Said heterocycloalkyl group can be e.g. oxetanyl, tetrahydrofuranyl, tetrahydropyranyl, piperidinyl, piperazinyl, or morpholinyl, particularly oxetanyl.

The terms “4- to 7-membered heterocycloalkyl” and “4- to 6-membered heterocycloalkyl” mean a monocyclic, saturated heterocycle with 4, 5, 6 or 7 or, respectively, 4, 5 or 6 ring atoms in total, which contains one or two identical or different ring heteroatoms from the series N, 0 and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as oxetanyl, tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, a heterocycloalkyl group means a “4- to 6-membered heterocycloalkyl” meaning a 4- to 6-membered heterocycloalkyl as defined supra containing one ring nitrogen atom or an oxygen atom. More particularly, “4- or 6-membered heterocycloalkyl” means a monocyclic, saturated heterocycle with 4 to 6 ring atoms in total, containing one of two oxygen atom, even more particularly heterocycloalkyl means oxetan-3-yl.

The term “fused heterocycloalkyl” means a bicyclic, saturated or partially heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl or any partially unsaturated derivative thereof.

The term “aryl” means a monocyclic or bicyclic aromatic group, particularly a phenyl group or a naphthyl group, more particularly a phenyl group. It includes also partially saturated groups such as e.g. dihydrophenyl, tetrahydrophenyl, indanyl or tetrahydronaphthyl. Possible substituents could be placed at any position chemically addressable either in the aromatic part or in the non-aromatic part of the residue.

The term “partially saturated monocyclic or bicyclic aryl group” means any group as defined for the term “aryl” of which one or two double bonds are hydrogenated, possibly substituted further with a C₁-C₄-alkyl group or a halogen atom.

The term “heteroaryl” means a monovalent, monocyclic, or bicyclic aromatic ring having 5, 6, 8, 9, 10, ring atoms (a “5- to 10-membered heteroaryl” group), which contains at least one ring heteroatom and optionally one or two further ring heteroatoms from the series: N, 0 and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly, the heteroaryl group is a furanyl group, a pyrolyl group, thiophenyl group, a pyrazolyl group, a oxazolyl group, a thiazolyl group, a pyridyl group, a benzimidazolyl group, a benzothiazolyl group and a isoquinolinyl group.

Where not mentioned separately the term “heteroaryl” includes partially saturated ring systems such as e.g. dihydrofuranyl, dihydrothiophenyl, dihydropyrrolyl, dihydropyrazolyl, dihydrooxazolyl, dihydropyridyl, tetrahydropyridyl, dihydroquinolinyl, tetrahydroquinolinyl, dihydroisoquinolinyl, tetrahydroisoquinolinyl, dihydrobenzopyranyl.

The term “partially saturated monocyclic or bicyclic heteroaryl group” includes e.g. dihydrofuranyl, dihydrothiophenyl, dihydropyrrolyl, dihydropyrazolyl, dihydrooxazolyl, dihydropyridyl, tetrahydropyridyl, dihydroquinolinyl, tetrahydroquinolinyl, dihydroisoquinolinyl, tetrahydroisoquinolinyl, dihydrobenzopyranyl

The term “C₁-C₆”, as used in the present text, e.g. in the context of the definition of e.g. “C₁-C₆-alkyl”, “C₁-C₆-haloalkyl”, “C₁-C₆-hydroxyalkyl”, or “C₁-C₆-alkoxy” means an alkyl group having a finite number of carbon atoms of 1 to 6, i.e. 1, 2, 3, 4, 5 or 6 carbon atoms. Any definitions deviating from this definition only in chain length are considered to be encompassed by the definition e.g. C₁-C₄-alkyl is encompassed by the definition of C₁-C₆-alkyl.

Further, as used herein, the term “C₃-C₆”, as used in the present text, e.g. in the context of the definition of “C₃-C₆-cycloalkyl”, means a cycloalkyl group having a finite number of carbon atoms of 3 to 8, i.e. 3, 4, 5 or 6 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₆” encompasses C₃, C₄, C₅, C₆, C₃-C₆, C₃-C₅, C₃-C₄, C₄-C₆, C₄-C₅, and C₅-C₆;

“C₄-C₆” encompasses C₄, C₅, C₆, C₄-C₆, C₄-C₅ and C₅-C₆;

“C₄-C₆” encompasses C₄, C₅, C₄-C₆, C₄-C₅, and C₅-C₆;

As used herein, the term “leaving group” means an atom or a group of atoms that is displaced in a chemical reaction as stable species taking with it the bonding electrons. In the particular situations of the present invention, such a leaving group is a halide, in particular a chloride, a bromide or a iodide, more particularly a bromide or a iodide, even more particularly a bromide.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

If only one isomer (enantiomer) displays the desired biological activity, and the second isomer (enantiomer) is inactive: Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem, 45,11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazol moiety as a heteroaryl group for example can exist as a 1H tautomer, or a 3H tautomer, or even a mixture in any amount of the two tautomers, namely:

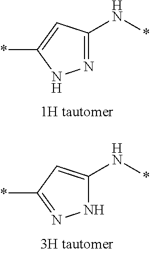 [SMILES: *Nc1cc(*)[nH]n1 ; *Nc1cc(*)n[nH]1]

The present invention includes all possible tautomers of the compounds of the present invention as single tautomers, or as any mixture of said tautomers, in any ratio.

The present invention also provides useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulf uric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethyl-ammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

### Further Embodiments of the Invention

In accordance with a further embodiment of the first aspect, the present invention provides compounds of general formula (I), supra, in which

R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted with a halogen atom, a C₁-C₄-alkoxy group, a C₁-C₄-alkyl group;

R² is selected from a hydrogen atom, a C₁-C₃-alkyl group, a —O—(CH₂)₂—NR⁸R⁹ group and a cyano group;

R³ is selected from a hydrogen atom, a cyano group, a C₁-C₃-alkyl group, a heterocycloalkyl group and a C₁-C₃-hydroxyalkyl group;

R⁴ is hydrogen;

R⁵ is selected from a hydrogen atom, a fluorine atom, a chlorine atom, a cyano group, a C₁-C₃-alkyl group, and a N(O)₂ group,

R⁶ is selected from a hydrogen atom, a fluorine atom, a chlorine atom, a C₁-C₃-alkyl group, and a C₂-C₄-alkynyl group,

R⁷ is selected from a hydrogen atom, a fluorine atom, and a C₁-C₃-alkyl group,

R⁸, R⁹ is independently selected from a hydrogen atom and a C₁-C₃-alkyl group;

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

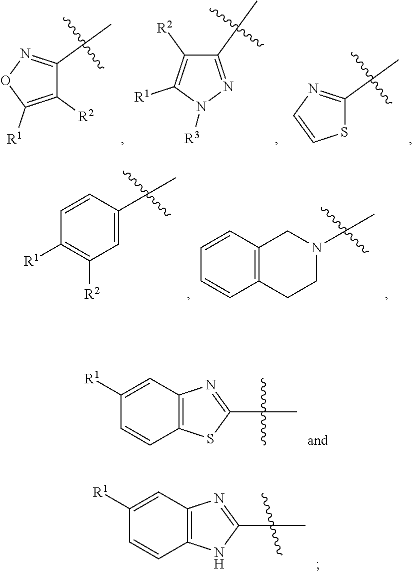

L¹ is selected from ^{$}—NH—C(O)—^{$$}, ^{$}—(CH₂)₃—NH—C(O)—^{$$}, ^{$}—(CH₂)₄—NH—C(O)—^{$$},

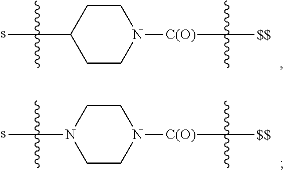

^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

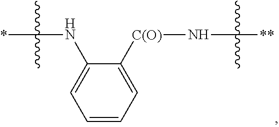

—CH₂-; and * is the point of attachment of L² to the thiazole carbon atom and ** is the point of attachment of L² to ring B;

B is selected from

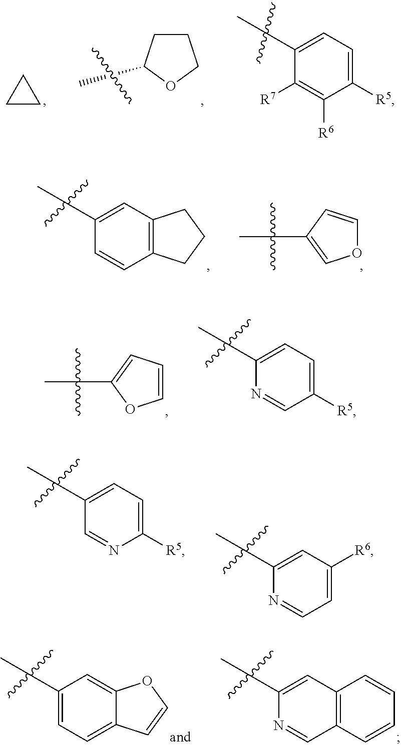

or a stereoisomer, a tautomer, a hydrate, a solvate, or a salt thereof, or a mixture of same.

In accordance with a further embodiment of the first aspect, the present invention provides compounds of general formula (I), supra, in which

R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted with a halogen atom, a C₁-C₄-alkoxy group, a C₁-C₄-alkyl group;

R² is selected from a hydrogen atom, a C₁-C₃-alkyl group, a —O—(CH₂)₂—NR⁸R⁹ group and a cyano group;

R³ is selected from a hydrogen atom, a cyano group, a C₁-C₃-alkyl group, a heterocycloalkyl group and a C₁-C₃-hydroxyalkyl group;

R⁴ is hydrogen;

R⁵ is selected from a hydrogen atom, a fluorine atom, a chlorine atom, a cyano group, a C₁-C₃-alkyl group, and a N(O)₂ group,

R⁶ is selected from a hydrogen atom, a fluorine atom, a chlorine atom, a C₁-C₃-alkyl group, and a C₂-C₄-alkynyl group,

R⁷ is selected from a hydrogen atom, a fluorine atom, and a C₁-C₃-alkyl group,

R⁸, R⁹ is independently selected from a hydrogen atom and a C₁-C₃-alkyl group;

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

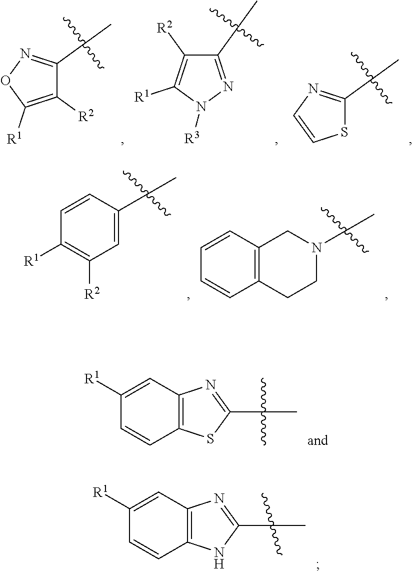

L¹ is selected from ^{$}—NH—C(O)—^{$$}, ^{$}—(CH₂)₃—NH—C(O)—^{$$}, ^{$}—(CH₂)₄—NH—C(O)—^{$$},

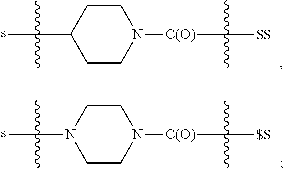

^{$} is the point of attachment of L¹ to ring A and ^{$$} is the point of attachment of L¹ to the thiazole carbon atom;

L² is selected from a bond, —NH—, *—NH—CH₂—**, *—NH—(CH₂)₂—**,

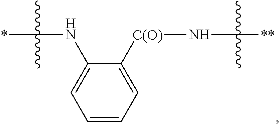

CH₂—; and * is the point of attachment of L² to the thiazole carbon atom and ** is the point of attachment of L² to ring B;

B is selected from

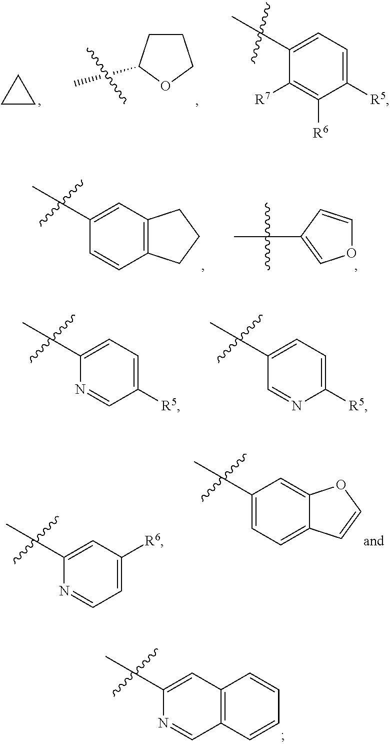

or a stereoisomer, a tautomer, a hydrate, a solvate, or a salt thereof, or a mixture of same.

In accordance with an embodiment of the a first aspect, the present invention provides compounds of general formula (I) wherein

R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted a C₁-C₄-alkyl group;

R² is selected from a hydrogen atom, a C₁-C₆-alkyl group, a —O—(CH₂)₂—NR⁸R⁹ group and a cyano group;

R³ is selected from a hydrogen atom, a cyano group, a NR⁸R⁹ group, a C₁-C₆-alkyl group, a C₁-C₆-hydroxyalkyl group and a heterocycloalkyl group;

R⁴ is hydrogen;

R⁵ is selected from a hydrogen atom, a halogen atom, a cyano group, a C₁-C₆-alkyl group and a N(O)₂ group;

R⁶ is selected from a hydrogen atom, a halogen atom;

R⁷ is selected from a hydrogen atom

R⁸, R⁹ is independently selected from a hydrogen atom and a C₁-C₆-alkyl group;

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

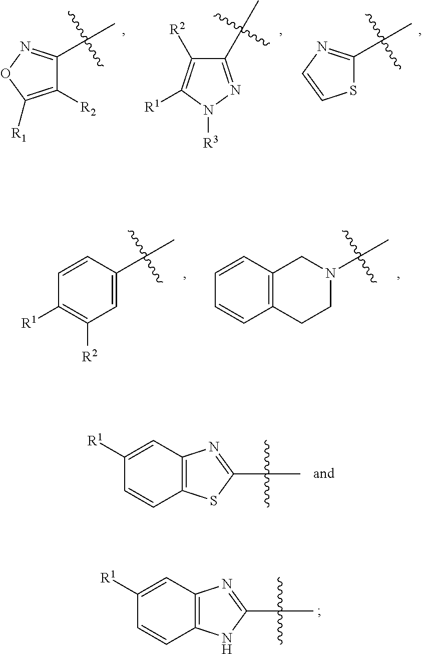

L¹ is selected from ^{$}—NH—C(O)—^{$$}, ^{$}—(CH₂)₃—NH—C(O)—^{$$}, ^{$}—(CH₂)₄—NH—C(O)—^{$$},

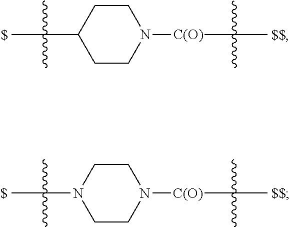

^{$} is the point of attachment of L¹ to ring A and ^{$$} is the point of attachment of L¹ to the thiazole carbon atom;

L² is —NH—; and * is the point of attachment of L² to the thiazole carbon atom and ** is the point of attachment of L² to ring B;

B is

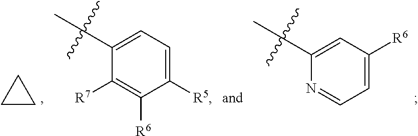

or a stereoisomer, a tautomer, a hydrate, a solvate, or a salt thereof, or a mixture of same.

In accordance with a further embodiment of the first aspect, the present invention provides compounds of general formula (I), supra, which are selected from:

or a stereoisomer, a tautomer, a hydrate, a solvate, or a salt thereof, or a mixture of same.

In accordance with a further embodiment of the first aspect, the present invention provides compounds of general formula (I), supra, which are selected from:

2-[(4-Chlorophenyl)amino]-N-(5-phenyl-1H-pyrazol-3-yl)-1,3-thiazole-4-carboxamide,

N-(5-tert-Butyl-1,2-oxazol-3-yl)-2-(pyridin-2-ylamino)-1,3-thiazole-4-carboxamide,

N-(5-tert-Butyl-1,2-oxazol-3-yl)-2-(phenylamino)-1,3-thiazole-4-carboxamide,

N-(5-tert-Butyl-1,2-oxazol-3-yl)-2-[(4-chlorophenyl)amino]-1,3-thiazole-4-carboxamide,

2-[(4-Chlorophenyl)amino]-N-(3-cyclopropyl-1H-pyrazol-5-yl)-1,3-thiazole-4-carboxamide,

N-(5-tert-Butyl-1,2-oxazol-3-yl)-2-[(5-fluoropyridin-2-yl)amino]-1,3-thiazole-4-carboxamide,

N-(5-tert-Butyl-1,2-oxazol-3-yl)-2-[(4-fluorophenyl)amino]-1,3-thiazole-4-carboxamide,

N-(1,3-Benzothiazol-2-yl)-2-[(4-chlorophenyl)amino]-1,3-thiazole-4-carboxamide,

2-[(4-Chlorophenyl)amino]-N-(5-methoxy-1H-benzimidazol-2-yl)-1,3-thiazole-4-carboxamide,

N-[1-Methyl-5-(trifluoromethyl)-1H-pyrazol-3-yl]-2-(phenylamino)-1,3-thiazole-4-carboxamide,

2-[(4-Chlorophenyl)amino]-N-(1,3-thiazol-2-yl)-1,3-thiazole-4-carboxamide,

N-(5-tert-Butyl-1H-pyrazol-3-yl)-2-[(4-chlorophenyl)amino]-1,3-thiazole-4-carboxamide,

N-(5-tert-Butyl-1,2-oxazol-3-yl)-2-[(4-nitrophenyl)amino]-1,3-thiazole-4-carboxamide,

N-(3-Cyclopropyl-1H-pyrazol-5-yl)-2-(phenylamino)-1,3-thiazole-4-carboxamide,

2-[(4-Chlorophenyl)amino]-N-(1-ethyl-1H-pyrazol-3-yl)-1,3-thiazole-4-carboxamide,

N-(5-tert-Butyl-1H-pyrazol-3-yl)-2-(phenylamino)-1,3-thiazole-4-carboxamide,

2-[(4-Chlorophenyl)amino]-N-[(4-(3,4-dihydroisoquinolin-2(1H)-yl)butyl]-1,3-thiazole-4-carboxamide,

N-(5-tert-Butyl-1,2-oxazol-3-yl)-2-[(3-chloro-4-methylphenyl)amino]-1,3-thiazole-4-carboxamide,

2-[(4-Chlorophenyl)amino]-N-(1,2-oxazol-3-yl)-1,3-thiazole-4-carboxamide,

2-[(4-Chlorophenyl)amino]-N-(5-methyl-1,2-oxazol-3-yl)-1,3-thiazole-4-carboxamide,

N-(5-tert-Butyl-1,2-oxazol-3-yl)-2-[(3,4-dichlorophenyl)amino]-1,3-thiazole-4-carboxamide,

N-{2-[2-(Diethylamino)ethoxy]-4′-methylbiphenyl-4-yl}-2-(phenylamino)-1,3-thiazole-4-carboxamide,

N-(1-Methyl-1H-pyrazol-3-yl)-2-(phenylamino)-1,3-thiazole-4-carboxamide,

N-(5-tert-Butyl-1,2-oxazol-3-yl)-2-[(4-cyanophenyl)amino]-1,3-thiazole-4-carboxamide,

N-(5-tert-Butyl-1,2-oxazol-3-yl)-2-[(2-fluorophenyl)amino]-1,3-thiazole-4-carboxamide,

2-[(4-Chlorophenyl)amino]-N-[3-(3,4-dihydroisoquinolin-2(1H)-yl)propyl]-1,3-thiazole-4-carboxamide,

{2-[(4-Chlorophenyl)amino]-1,3-thiazol-4-yl}{4-[3,4-dihydroisoquinolin-2(1H)-yl]piperidin-1-yl}methanone,

N-[1-(2-Hydroxyethyl)-1H-pyrazol-3-yl]-2-(phenylamino)-1,3-thiazole-4-carboxamide,

N-(5-tert-Butyl-4-methyl-1,2-oxazol-3-yl)-2-[(4-chlorophenyl)amino]-1,3-thiazole-4-carboxamide,

N-(4-Cyano-1H-pyrazol-3-yl)-2-(phenylamino)-1,3-thiazole-4-carboxamide,

2-[(4-Chlorophenyl)amino]-N-[1-(oxetan-3-yl)-1H-pyrazol-4-yl]-1,3-thiazole-4-carboxamide,

N-(5-tert-Butyl-1,2-oxazol-3-yl)-2-(cyclopropylamino)-1,3-thiazole-4-carboxamide, and

{2-[(4-Chlorophenyl)amino]-1,3-thiazol-4-yl}[4-(5-methyl-1,3-benzothiazol-2-yl)piperazin-1-yl]methanone

or a stereoisomer, a tautomer, a hydrate, a solvate, or a salt thereof, or a mixture of same.

In accordance with a further embodiment of the first aspect, the present invention provides compounds of general formula (I), supra, which are selected from:

N-(5-tert-Butyl-1,2-oxazol-3-yl)-2-[(5-methylpyridin-2-yl)amino]-1,3-thiazole-4-carboxamide,

N-(5-tert-Butyl-1,2-oxazol-3-yl)-2-[(5-chloropyridin-2-yl)amino]-1,3-thiazole-4-carboxamide,

N-(5-tert-Butyl-1,2-oxazol-3-yl)-2-[(4-chloropyridin-2-yl)amino]-1,3-thiazole-4-carboxamide,

N-(5-tert-Butyl-1,2-oxazol-3-yl)-2-[(2-phenylethyl)amino]-1,3-thiazole-4-carboxamide,

N-(5-tert-Butyl-1,2-oxazol-3-yl)-2-(2,3-dihydro-1H-inden-5-ylamino)-1,3-thiazole-4-carboxamide,

N-(5-tert-Butyl-1,2-oxazol-3-yl)-2-[(4-chloro-3-fluorophenyl)amino]-1,3-thiazole-4-carboxamide,

2-(1-Benzofuran-6-ylamino)-N-(5-tert-butyl-1,2-oxazol-3-yl)-1,3-thiazole-4-carboxamide,

N-(5-tert-Butyl-1,2-oxazol-3-yl)-2-[(2-fluoro-4-methylphenyl)amino]-1,3-thiazole-4-carboxamide,

N-(5-tert-Butyl-1,2-oxazol-3-yl)-2-[(3-fluorobenzyl)amino]-1,3-thiazole-4-carboxamide,

N-(5-tert-Butyl-1,2-oxazol-3-yl)-2-[(3-methylphenyl)amino]-1,3-thiazole-4-carboxamide,

N-(5-tert-Butyl-1,2-oxazol-3-yl)-2-[(furan-2-ylmethyl)amino]-1,3-thiazole-4-carboxamide,

N-(5-tert-Butyl-1,2-oxazol-3-yl)-2-[(3,4-difluorophenyl)amino]-1,3-thiazole-4-carboxamide,

N-(5-tert-Butyl-1,2-oxazol-3-yl)-2-[(3-chloro-4-fluorophenyl)amino]-1,3-thiazole-4-carboxamide,

N-(5-tert-Butyl-1,2-oxazol-3-yl)-2-{[2-(isoquinolin-3-ylcarbamoyl)phenyl]amino}-1,3-thiazole-4-carboxamide,

N-(5-tert-Butyl-1,2-oxazol-3-yl)-2-[(3-chlorophenyl)amino]-1,3-thiazole-4-carboxamide,

(rac)-N-(5-tert-Butyl-1,2-oxazol-3-yl)-2-{[-tetrahydrofuran-2-ylmethyl]amino}-1,3-thiazole-4-carboxamide,

N-(5-tert-Butyl-1,2-oxazol-3-yl)-2-[(4-fluoro-2-methylphenyl)amino]-1,3-thiazole-4-carboxamide,

N-(5-tert-Butyl-1,2-oxazol-3-yl)-2-[(3-ethynylphenyl)amino]-1,3-thiazole-4-carboxamide,

N-(5-tert-Butyl-1,2-oxazol-3-yl)-2-[(2,3-difluorophenyl)amino]-1,3-thiazole-4-carboxamide and

N-(5-tert-Butyl-1,2-oxazol-3-yl)-2-[(6-fluoropyridin-3-yl)amino]-1,3-thiazole-4-carboxamide

or a stereoisomer, a tautomer, a hydrate, a solvate, or a salt thereof, or a mixture of same.

In further embodiments, the present invention provides compounds of formula (I), supra, in which:

R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted with a halogen atom, a C₁-C₄-alkoxy group, a C₁-C₄-alkyl group; and stereoisomers, tautomers, hydrates, solvates, and salts thereof, and mixtures of same.

In further embodiments, the present invention provides compounds of formula (I), supra, in which:

R¹ is selected from a hydrogen atom, a halogen atom, a C₁-C₄-alkyl group, a C₁-C₄-haloalkyl group, a C₃-C₆-cycloalkyl group, a C₁-C₄-alkoxy group and a phenyl group which is optionally substituted with a halogen atom, a C₁-C₄-alkoxy group, a C₁-C₄-alkyl group; and stereoisomers, tautomers, hydrates, solvates, and salts thereof, and mixtures of same.

In further embodiments, the present invention provides compounds of formula (I), supra, in which:

R¹ is selected from a hydrogen atom, a C₁-C₄-alkyl group, a C₁-C₃-haloalkyl group, a C₃-C₆-cycloalkyl group, a C₁-C₃-alkoxy group and a phenyl group which is optionally substituted with a C₁-C₃-alkyl group; and stereoisomers, tautomers, hydrates, solvates, and salts thereof, and mixtures of same.

In further embodiments, the present invention provides compounds of formula (I), supra, in which:

R¹ is selected from a hydrogen atom, a halogen atom, a C₁-C₃-alkyl group, a C₁-C₃-haloalkyl group, a C₃-C₆-cycloalkyl group, a C₁-C₃-alkoxy group and a phenyl group which is optionally substituted with a C₁-C₃-alkyl group; and stereoisomers, tautomers, hydrates, solvates, and salts thereof, and mixtures of same.

In further embodiments, the present invention provides compounds of formula (I), supra, in which:

R¹ is selected from a hydrogen atom, a halogen atom, a C₁-C₄-alkyl group, a C₁-C₂-haloalkyl group, a C₃-C₄-cycloalkyl group, a C₁-C₂-alkoxy group and a phenyl group which is optionally substituted with a C₁-C₂-alkyl group; and stereoisomers, tautomers, hydrates, solvates, and salts thereof, and mixtures of same.

In further embodiments, the present invention provides compounds of formula (I), supra, in which:

R¹ is selected from a hydrogen atom, a methyl group, a tert-butyl group, a trifluoromethyl group, a cyclopropyl group, a methoxy group, a phenyl group and a 4-methylphenyl group; and stereoisomers, tautomers, hydrates, solvates, and salts thereof, and mixtures of same. In yet further embodiments, the present invention provides compounds of formula (I), supra, in which:

R² is selected from a hydrogen atom, a C₁-C₆-alkyl group, a —O—(CH₂)₂—NR⁸R⁹ group and a cyano group and R⁸, R⁹ are independently selected from a hydrogen atom and a C₁-C₆-alkyl group; and stereoisomers, tautomers, hydrates, solvates, and salts thereof, and mixtures of same.

In further embodiments, the present invention provides compounds of formula (I), supra, in which:

R² is selected from a hydrogen atom, a C₁-C₃-alkyl group, a —O—(CH₂)₂—NR⁸R⁹ group and a cyano group and R⁸, R⁹ are independently selected from a hydrogen atom and a C₁-C₃-alkyl group; and stereoisomers, tautomers, hydrates, solvates, and salts thereof, and mixtures of same.

In other embodiments, the present invention provides compounds of formula (I), supra, in which:

R² is selected from a hydrogen atom, a methyl group, a —O—(CH₂)₂—N(CH₂CH3)2 group and a cyano group and stereoisomers, tautomers, hydrates, solvates, and salts thereof, and mixtures of same.

In further embodiments, the present invention provides compounds of formula (I), supra, in which:

R³ is selected from a hydrogen atom, a cyano group, a NR⁸R⁹ group, a C₁-C₆-alkyl group, a C₁-C₆-hydroxyalkyl group and a heterocycloalkyl group and stereoisomers, tautomers, hydrates, solvates, and salts thereof, and mixtures of same.

In further embodiments, the present invention provides compounds of formula (I), supra, in which:

R³ is selected from a hydrogen atom, a cyano group, a NR⁸R⁹ group, a C₁-C₃-alkyl group, a C₁-C₃-hydroxyalkyl group and a heterocycloalkyl group and stereoisomers, tautomers, hydrates, solvates, and salts thereof, and mixtures of same.

In further embodiments, the present invention provides compounds of formula (I), supra, in which:

R³ is selected from a hydrogen atom, a cyano group, a NR⁸R⁹ group, a C₁-C₃-alkyl group, a C₁-C₃-hydroxyalkyl group and a heterocycloalkyl group and stereoisomers, tautomers, hydrates, solvates, and salts thereof, and mixtures of same.

In further embodiments, the present invention provides compounds of formula (I), supra, in which:

R³ is selected from a hydrogen atom, a cyano group, a NR⁸R⁹ group, a C₁-C₂-alkyl group, a C₁-C₂-hydroxyalkyl group and a 1,4-oxetanyl group and stereoisomers, tautomers, hydrates, solvates, and salts thereof, and mixtures of same.

In further embodiments, the present invention provides compounds of formula (I), supra, in which:

R⁵ is selected from a hydrogen atom, a halogen atom, a cyano group, a C₁-C₆-alkyl group and a N(O)₂ group and stereoisomers, tautomers, hydrates, solvates, and salts thereof, and mixtures of same.

In further embodiments, the present invention provides compounds of formula (I), supra, in which:

R⁵ is selected from a hydrogen atom, a halogen atom, a cyano group, a C₁-C₃-alkyl group and a N(O)₂ group and stereoisomers, tautomers, hydrates, solvates, and salts thereof, and mixtures of same.

In further embodiments, the present invention provides compounds of formula (I), supra, in which:

R⁵ is selected from a hydrogen atom, a fluorine atom, a chlorine atom, a cyano group, a C₁-C₃-alkyl group and a N(O)₂ group and stereoisomers, tautomers, hydrates, solvates, and salts thereof, and mixtures of same.

In further embodiments, the present invention provides compounds of formula (I), supra, in which:

R⁵ is selected from a hydrogen atom, a fluorine atom, a chlorine atom, a cyano group, a methyl group and a N(O)₂ group and stereoisomers, tautomers, hydrates, solvates, and salts thereof, and mixtures of same.

In further embodiments, the present invention provides compounds of formula (I), supra, in which:

R⁵ is selected from a hydrogen atom, a fluorine atom, a chlorine atom, a cyano group and a methyl group and stereoisomers, tautomers, hydrates, solvates, and salts thereof, and mixtures of same.

In further embodiments, the present invention provides compounds of formula (I), supra, in which:

R⁶ is selected from a hydrogen atom, a halogen atom, a C₁-C₆-alkyl group, a C₂-C₆-alkenyl group and a C₂-C₆-alkynyl group; and stereoisomers, tautomers, hydrates, solvates, and salts thereof, and mixtures of same.

In further embodiments, the present invention provides compounds of formula (I), supra, in which:

R⁶ is selected from a hydrogen atom, a halogen atom, a C₁-C₃-alkyl group, a C₂-C₃-alkenyl group and a C₂-C₃-alkynyl group; and stereoisomers, tautomers, hydrates, solvates, and salts thereof, and mixtures of same.

In further embodiments, the present invention provides compounds of formula (I), supra, in which:

R⁶ is selected from a hydrogen atom, a halogen atom, a C₁-C₃-alkyl group and a C₂-C₃-alkynyl group; and stereoisomers, tautomers, hydrates, solvates, and salts thereof, and mixtures of same.

In further embodiments, the present invention provides compounds of formula (I), supra, in which:

R⁶ is selected from a hydrogen atom, a fluorine atom, a chlorine atom, a methyl group and a ethynyl group and stereoisomers, tautomers, hydrates, solvates, and salts thereof, and mixtures of same.

In further embodiments, the present invention provides compounds of formula (I), supra, in which:

R⁷ is selected from a hydrogen atom, a halogen atom and a C₁-C₆-alkyl group and stereoisomers, tautomers, hydrates, solvates, and salts thereof, and mixtures of same.

In further embodiments, the present invention provides compounds of formula (I), supra, in which:

R⁷ is selected from a hydrogen atom, a fluorine atom, a chlorine atom and a C₁-C₆-alkyl group and stereoisomers, tautomers, hydrates, solvates, and salts thereof, and mixtures of same.

In further embodiments, the present invention provides compounds of formula (I), supra, in which:

R⁷ is selected from a hydrogen atom, a fluorine atom and a methyl group and stereoisomers, tautomers, hydrates, solvates, and salts thereof, and mixtures of same.

In further embodiments, the present invention provides compounds of formula (I), supra, in which:

R⁸, R⁹ is independently selected from a hydrogen atom and a C₁-C₆-alkyl group and stereoisomers, tautomers, hydrates, solvates, and salts thereof, and mixtures of same.

In yet further embodiments, the present invention provides compounds of formula (I), supra, in which:

R⁸, R⁹ is independently selected from a hydrogen atom and a C₁-C₃-alkyl group and stereoisomers, tautomers, hydrates, solvates, and salts thereof, and mixtures of same.

In further embodiments, the present invention provides compounds of formula (I), supra, in which:

R⁸, R⁹ is independently selected from a hydrogen atom and a C₁-C₂-alkyl group and stereoisomers, tautomers, hydrates, solvates, and salts thereof, and mixtures of same.

In yet further embodiments, the present invention provides compounds of formula (I), supra, in which:

R⁸, R⁹ is independently selected from a hydrogen atom and a ethyl group and stereoisomers, tautomers, hydrates, solvates, and salts thereof, and mixtures of same.

In yet further embodiments, the present invention provides compounds of formula (I), supra, in which:

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

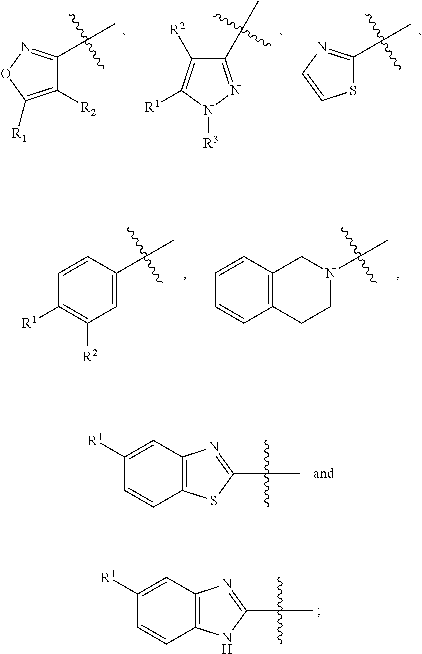

and stereoisomers, tautomers, hydrates, solvates, and salts thereof, and mixtures of same.

In yet further embodiments, the present invention provides compounds of formula (I), supra, in which:

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

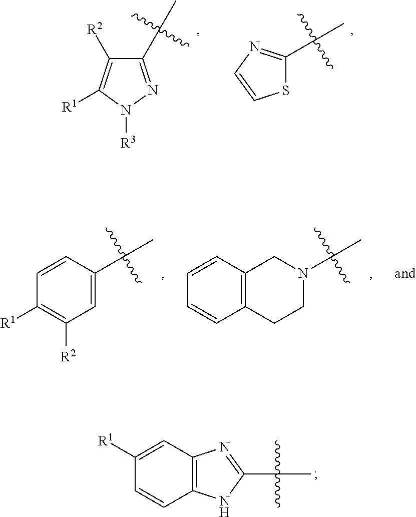

and stereoisomers, tautomers, hydrates, solvates, and salts thereof, and mixtures of same.

In yet further embodiments, the present invention provides compounds of formula (I), supra, in which:

A is selected from a monocyclic or bicyclic aryl group, a partially saturated monocyclic or bicyclic aryl group, a monocyclic or bicyclic heteroaryl group, a partially saturated monocyclic or bicyclic heteroaryl group,

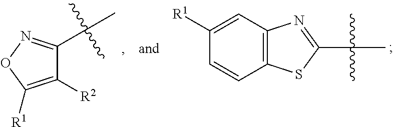

and stereoisomers, tautomers, hydrates, solvates, and salts thereof, and mixtures of same.

In yet further embodiments, the present invention provides compounds of formula (I), supra, in which

L¹ is selected from ^{$}—NH—C(O)—^{$$}, ^{$}—(CH₂)₃—NH—C(O)—^{$$}, ^{$}—(CH₂)₄—NH—C(O)—^{$$},

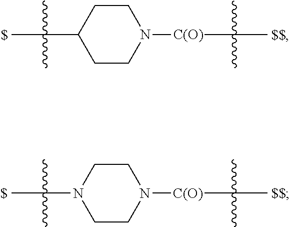

^{$} is the point of attachment of L¹ to ring A and ^{$$} is the point of attachment of L¹ to the thiazole carbon atom; and stereoisomers, tautomers, hydrates, solvates, and salts thereof, and mixtures of same.

In yet further embodiments, the present invention provides compounds of formula (I), supra, in which

L¹ is from ^{$}—NH—C(O)—^{$$};
^{$} is the point of attachment of L¹ to ring A and ^{$$} is the point of attachment of L¹ to the thiazole carbon atom; and stereoisomers, tautomers, hydrates, solvates, and salts thereof, and mixtures of same.

In yet further embodiments, the present invention provides compounds of formula (I), supra, in which

L² is selected from a bond, —NH—, *—NH—CH₂—**, *—NH—(CH₂)₂—**,

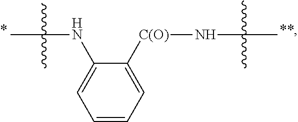

—CH₂-; and * is the point of attachment of L² to the thiazole carbon atom and ** is the point of attachment of L² to ring B; and stereoisomers, tautomers, hydrates, solvates, and salts thereof, and mixtures of same.

In yet further embodiments, the present invention provides compounds of formula (I), supra, in which

L² is —NH—; and * is the point of attachment of L² to the thiazole carbon atom and ** is the point of attachment of L² to ring B;

and stereoisomers, tautomers, hydrates, solvates, and salts thereof, and mixtures of same.

In yet further embodiments, the present invention provides compounds of formula (I), supra, in which

B is selected from

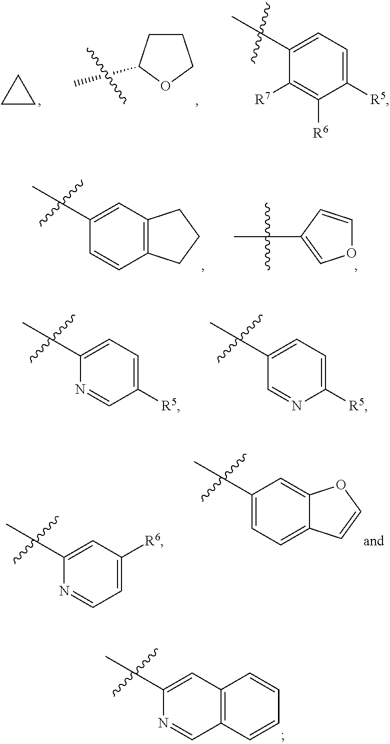

or a stereoisomer, a tautomer, a hydrate, a solvate, or a salt thereof, or a mixture of same.

In yet further embodiments, the present invention provides compounds of formula (I), supra, in which

B is selected from

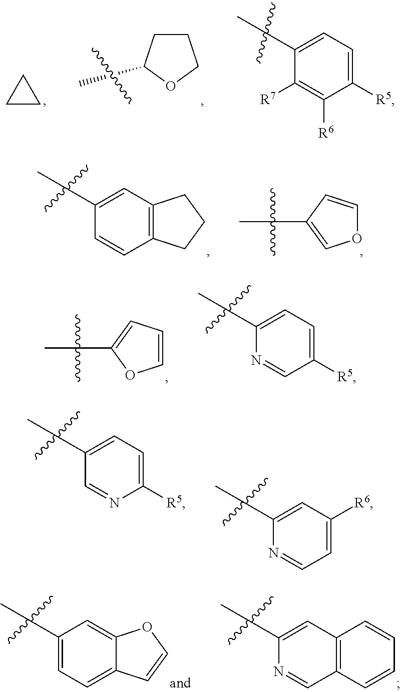

or a stereoisomer, a tautomer, a hydrate, a solvate, or a salt thereof, or a mixture of same.

In further embodiments, the present invention provides compounds of formula (I), supra, in which

B is selected from

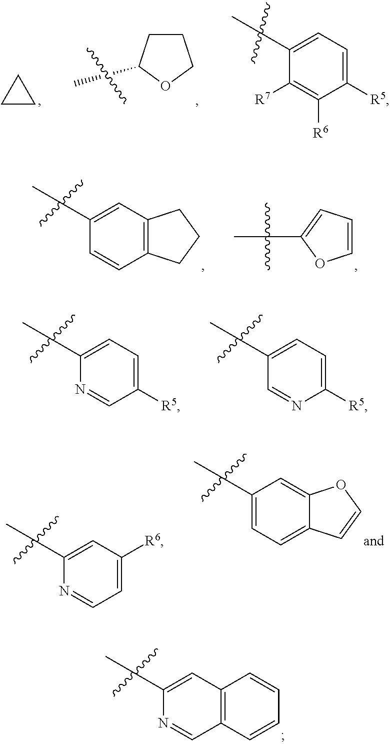

or a stereoisomer, a tautomer, a hydrate, a solvate, or a salt thereof, or a mixture of same.

In yet further embodiments, the present invention provides compounds of formula (I), supra, in which

B is selected from

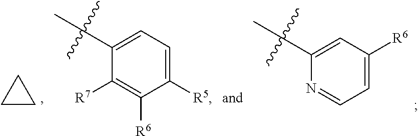

or a stereoisomer, a tautomer, a hydrate, a solvate, or a salt thereof, or a mixture of same.

In yet further embodiments, the present invention provides compounds of formula (I), supra, in which

B is selected from

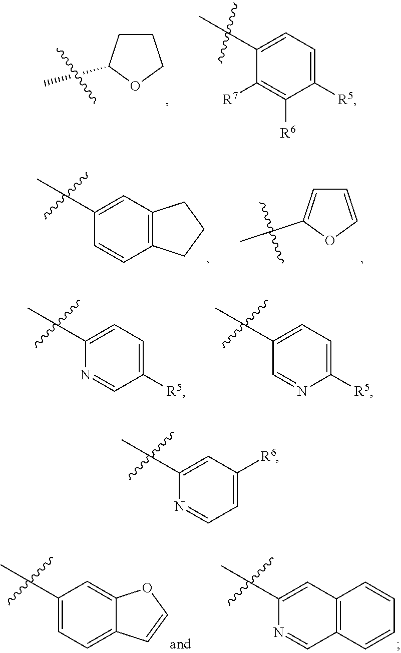

or a stereoisomer, a tautomer, a hydrate, a solvate, or a salt thereof, or a mixture of same.

In yet further embodiments, the present invention provides compounds of formula (I), supra, in which

B is selected from

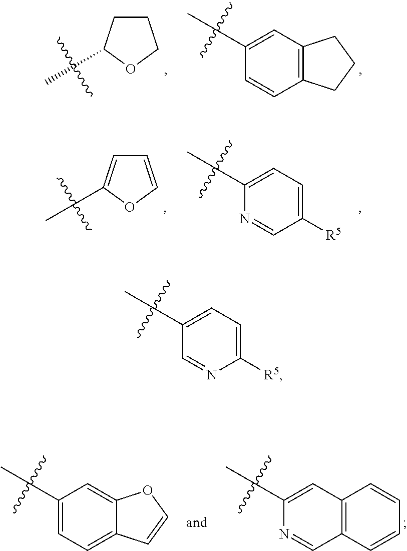

or a stereoisomer, a tautomer, a hydrate, a solvate, or a salt thereof, or a mixture of same.

In particular further embodiments, the present invention provides combinations of two or more of the above mentioned embodiments under the heading “further embodiments”.

In accordance with a further embodiment of the first aspect, the present invention provides compounds of general formula (I), supra, in which

R⁶ is selected from a C₁-C₆-alkyl group, and a C₂-C₆-alkynyl group;

L² is selected from *—NH—CH₂—**, *—NH—(CH₂)₂—**,

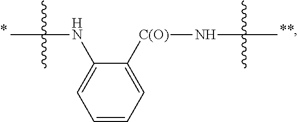

and * is the point of attachment of L² to the thiazole carbon atom and ** is the point of attachment of L² to ring B;

B is selected from

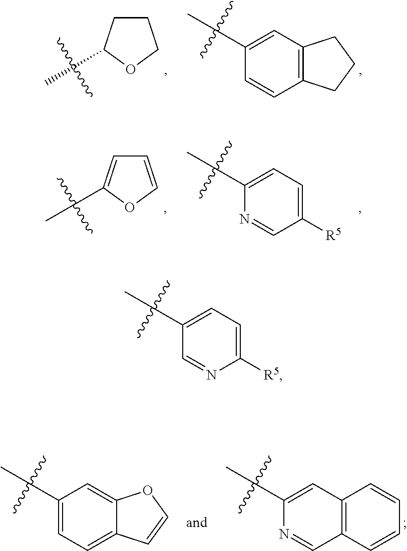

or a stereoisomer, a tautomer, a hydrate, a solvate, or a salt thereof, or a mixture of same.

The present invention provides any sub-combination within any embodiment or aspect of the present invention of compounds of general formula (I), supra.

The present invention provides any sub-combination within any embodiment or aspect of the present invention of intermediate compounds of general formulae (II), (III), (IV) and (V).

The present invention provides the compounds of general formula (I) which are disclosed in the Example Section of this text, infra

General Syntheses of Compounds of Formula (I)

The compounds according to the invention of general formula (I) can be prepared according to the following schemes 1, 2 and 3. The schemes and procedures described below illustrate synthetic routes to the compounds of general formula (I) of the invention and are not intended to be limiting. It is clear to the person skilled in the art that the order of transformations as exemplified in schemes 1, 2 and 3 can be modified in various ways. The order of transformations exemplified in these schemes is therefore not intended to be limiting. In addition, interconversion of any of the substituents, R¹, R², R³, R⁴, L¹ or L² can be achieved before and/or after the exemplified transformations. These modifications can be such as the introduction of protecting groups, cleavage of protecting groups, reduction or oxidation of functional groups, halogenation, metallization, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Three routes for the preparation of compounds of general formula (I) are described in schemes 1, 2 and 3.

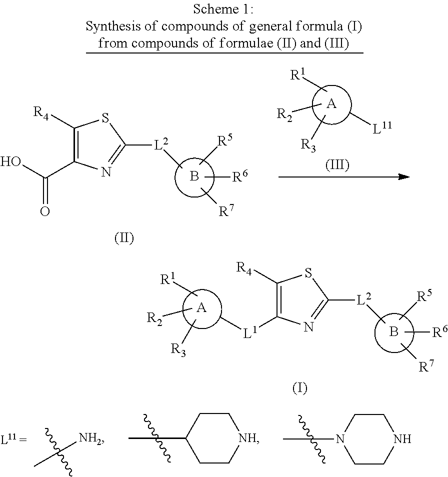

As outlined in Scheme 1, compounds of general formula (I) can be prepared by reacting starting materials of formulae (II) and (III), in which L¹¹ represents an amino group as depicted in Scheme 1 using a coupling agent well known to the person skilled in the art, such as e.g. O-(7-azabenzotriazol-1-yl)-N,N,N′,N′-tetramethyluronium hexafluorophosphate (HATU), 2,4,6-tripropyl-1,3,5,2,4,6-trioxatriphosphorinane-2,4,6-trioxide (T3P), O-(benzotriazol-1-yl)-N,N,N′,N′-tetramethyluronium tetrafluoroborate (TBTU), ({[(Z)-(1-cyano-2-ethoxy-2-oxoethylidene)amino]oxy}-N,N-dimethylmorpholin-4-ylmethaniminium hexafluorophosphate (COMU) or N,N′-dicyclohexyl carbodiimide (DCC) in the presence of a base, such as e.g. a tertiary aliphatic amine of the formula N(C₁-C₄-alkyl)₃, preferably N,N-diisopropylethylamine or triethylamine, in a solvent, particularly a dipolar aprotic solvent such as e.g. acetonitrile, N,N-dimethylformamide, N,N-dimethylacetamide or N-methylpyrrolidin-2-one, to give the carboxamide of formula general formula (I) at room temperature up to 40° C., preferably at room temperature. Compounds of formulae (II), (III), respectively, and methods for their preparation are well known to the person skilled in the art or are described in the experimental section. Further, compounds of formulae (II), (III) are commercially available in considerable structural variety.

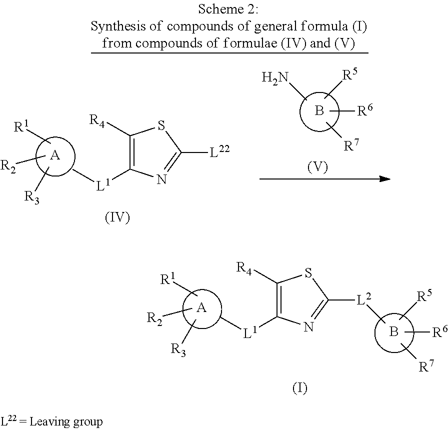

Alternatively according to Scheme 2, compounds of formula (IV), in which L²² represents a suitable leaving group, e.g. a chlorine, bromine, iodine atom, preferably a bromine atom, and in which R¹, R², R³, R⁴ and L¹ are as defined for the compounds of general formula (I), can be converted into the final compounds of general formula (I). Said conversion can be

a) either accomplished by nucleophilic substitution as described in the general procedure GP2, i.e. by reacting bromo compounds of formula (IV), in which L²² represents a suitable leaving group, such as a halogenide such as a chloride, a bromide or an iodide, more preferably a bromide, and in which R¹, R², R³, R⁴ and L¹ are as defined for the compounds of general formula (I), with an amine (V), in which R⁵, R⁶ and R⁷ are as defined for compounds of formula (I), in the presence of a base, such as sodium hydride or an alkali carbonate or alkali phosphate, e.g. sodium carbonate, potassium carbonate, cesium carbonate, sodium phosphate, potassium phosphate, preferably potassium carbonate, in the presence of a polar aprotic solvent such as e.g. a cyclic ether like tetrahydofurane (THF), 1,4-dioxane or dimethylsulfoxide at a temperature range between 60° C. and 80° C., preferably at 67° C.

b) by a palladium or copper catalyzed amination reaction, i.e. by reacting bromo compounds of formula (IV), in which L²² represents a suitable leaving group, such as a chloride, a bromide or an iodide, preferably a bromine atom and in which R¹, R², R³, R⁴ and L¹ are as defined for the compounds of general formula (IV), with an amine of general formula (V) in which R⁵, R⁶ and R⁷ are as defined for compounds of formula (I), in the presence of a palladium catalyst, such as e.g. tris(dibenzylideneacetone)dipalladium chloroform complex in combination with 9,9-dimethyl-4,5-bis(diphenylphosphino)xanthene as a ligand, and in the presence of a base, such as an alkali carbonate or alkali phosphate, preferably cesium carbonate or potassium phosphate, or in the presence of tris-(dibenzylideneacetone)dipalladium(0) in combination with (5-diphenylphosphanyl-9,9-dimethyl-xanthen-4-yl)-diphenyl-phosphane (WO2006/101977 A2, 2006) or

in the presence of a copper catalyst, such as e.g. copper(i) iodide or copper(ii) acetate, and in the presence of a base such aliphatic amines as 1,2-diaminocyclohexane, glycine, or proline or with alkali bases such as sodium carbonate, potassium carbonate, caesium carbonate, preferably cesium carbonate

in the presence of a solvent such as toluene, 1,4-dioxane, dimethylsulfoxide or N,N-dimethylformamide at a temperature between 60° C. and 130° C., using a sealed vessel if needed to arrive at compound of formula (I).

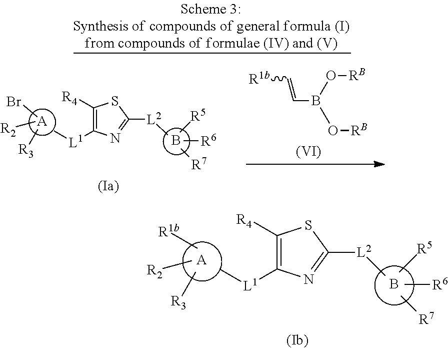

Further, according to Scheme 3, compounds of general formula (I) in which R², R³ R⁴, R⁵, R⁶, R⁷ L¹ and L² are as defined in claim 1 and R¹ is an optionally substituted phenyl group which is named as Rib could also be obtained by using palladium coupling reactions as Suzuki-Miyaura which is well known to the person skilled in the art by reaction with boronic acid derivatives of formula (VI), e.g. by reaction with phenylboronic acid pinacol ester in the presence of a palladium catalyst, such as commercially available tetrakis(triphenylphosphine)palladium, bis(triphenylphosphine)palladium(ii) dichloride or palladium(ii)chloride, a said boronic acid derivative of formula (VI) which may be a boronic acid (R^{B}═—H) or an ester of the boronic acid, e.g. its isopropyl ester (R^{B}═C₁-C₄-alkyl, e.g. —CH(CH₃)₂), or an ester derived from a diol such as pinacol in which the boronic acid intermediate forms a cyclic boronic ester, such as a 4,4,5,5-tetramethyl-1,3,2-dioxaborolane (R^{B}—R^{B}═C₂-C₆-alkylene, e.g. —C(CH₃)₂—C(CH₃)₂—) and for which R^{1b} means a optionally substituted phenyl group. Many boronic acids and their esters are commercially available and well-known to the person skilled in the art (see e.g. D. G. Hall, Boronic Acids, 2005 WILEY-VCH Verlag GmbH & Co. KGaA, Weinheim, ISBN 3-527-30991-8 and references cited therein) and a base, such as an alkali carbonate or an alkali phosphate, e.g. sodium carbonate, potassium carbonate, cesium carbonate, sodium phosphate, potassium phosphate, preferably sodium carbonate or amines such as trimethylamine, methyldiethylamine, methyl diisopropylamine, triethylamine, triisopropylamine, preferably triiethylamine in an polar aprotic solvent, such as e.g. an ether, or N,N-dimethylformamide, the ether being such as e.g. tetrahydrofuran, tetrahydropyran, 1,2-dimethoxyethane or 1,4-dioxan, at a temperature range between 60° C. and 130° C., preferably a sealed vessel if needed.

In accordance with a second aspect, the present invention provides methods of preparing compounds of general formula (I) as defined supra, said methods comprising the step of allowing an intermediate compound of general formula (II):

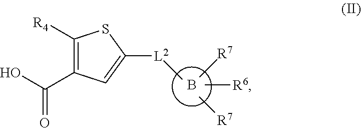

in which R⁴, R⁵, R⁶, R⁷ and L² are as defined for the compound of general formula (I) as defined supra,

to react with a compound of general formula (III):

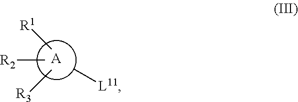

in which R¹, R², R³ are as defined for the compound of general formula (I) as defined supra, and L¹¹ is

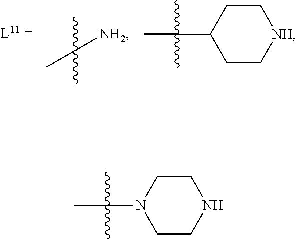

thereby giving a compound of general formula (I):

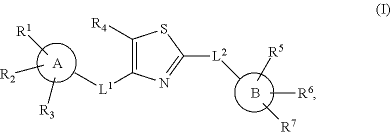

in which R¹, R², R³ R⁴, R⁵, R⁶, R⁷ L¹ and L² are as defined in claim 1.

In accordance with a third aspect, the present invention provides methods of preparing compounds of general formula (I) as defined supra, said methods comprising the step of allowing an intermediate compound of general formula (II):

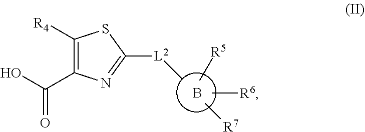

in which R⁴, R⁵, R⁶, R⁷ and L² are as defined for the compound of general formula (I) as defined supra,

to react with a compound of general formula (III):

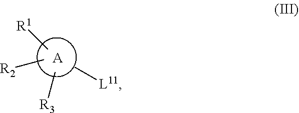

in which R¹, R², R³ are as defined for the compound of general formula (I) as defined supra, and L¹¹ is

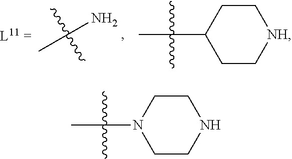

thereby giving a compound of general formula (I):

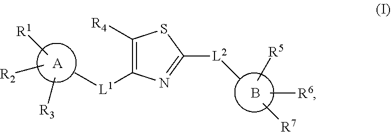

in which R¹, R², R³ R⁴, R⁵, R⁶, R⁷ L¹ and L² are as defined in claim 1.

then optionally removing any protecting groups used and optionally converting said compound into solvates, salts and/or solvates of such salts using the corresponding (i) solvents and/or (ii) bases or acids.

In accordance with a fourth aspect, the present invention provides methods of preparing compounds of general formula (I) as defined supra, said methods comprising the step of allowing an intermediate compound of general formula (IV)

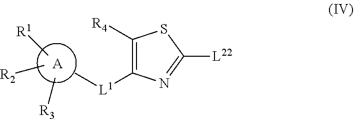

in which R¹, R², R³, R⁴ and L¹ are as defined for the compound of general formula (I) as defined supra,

L²² is a suitable leaving group, such as a halogenide such as a chloride, a bromide or a iodide, more preferably a bromide,

to react with a compound of general formula (V):

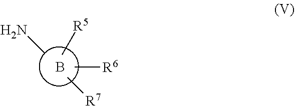

in which R⁵, R⁶, R⁷ and R⁴ are as defined for the compound of general formula (I) as defined supra, in the presence of a base or additionally in the presence of a palladium or copper catalyst,

thereby giving a compound of general formula (I):

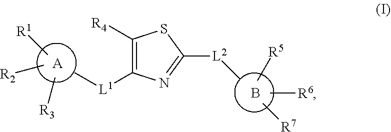

in which R¹, R², R³ R⁴, R⁵, R⁶, R⁷ L¹ and L² are as defined in claim 1,

then optionally removing any protecting groups used and optionally converting said compound into solvates, salts and/or solvates of such salts using the corresponding (i) solvents and/or (ii) bases or acids.

The present invention further provides methods of preparing compounds of the present invention of general formula (I), said methods comprising the steps as described in the Experimental Section herein.

In accordance with a fourth aspect, the present invention provides intermediate compounds which are useful for the preparation of the compounds of general formula (I), supra.

Particularly, the invention provides the intermediate compounds of general formula (II):

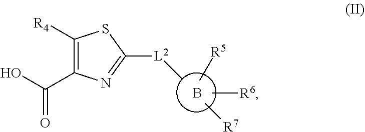

in which R⁴, R⁵, R⁶, R⁷ and L² are as defined for the compound of general formula (I) as defined supra.

In accordance with a fifth aspect, the present invention provides the use of said intermediate compounds for the preparation of a compound of general formula (I) as defined supra.

Particularly, the invention provides the use of intermediate compounds of general formula (II):

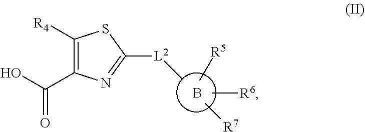

in which R⁴, R⁵, R⁶, R⁷ and L² are as defined for the compound of general formula (I) as defined supra, for the preparation of a compound of general formula (I) as defined supra.

Furthermore, the invention provides the intermediate compounds of general formula (II).

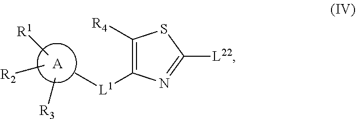

in which R¹, R², R³, R⁴ and L¹ are as defined for the compound of general formula (I) as defined supra and R²² is a suitable leaving group, such as halogenide such as a chloride, a bromide or a iodide, more preferably a bromide.

Particularly, the invention provides the use of intermediate compounds of general formula (IV):

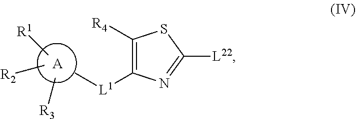

in which R¹, R², R³, R⁴ and L¹ are as defined for the compound of general formula (I) as defined supra, for the preparation of a compound of general formula (I) as defined supra and R²² is a suitable leaving group, such as a halogenide particularly a chloride, a bromide or a iodide, more preferably a bromide for the preparation of a compound of general formula (I) as defined supra.

Even further, the invention provides the intermediate compounds of general formulae (III) and (V):

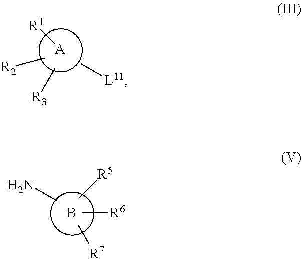

in which R¹, R², R³ are as defined for the compound of general formula (I) as defined supra, and L¹¹ is

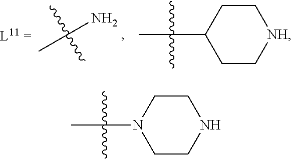

and

in which R⁵, R⁶, R⁷ and R⁴ are as defined for the compound of general formula (I) as defined supra.

Particularly, the invention provides the use of intermediate compounds of general formulae (III) and (V) as defined above for the preparation of a compound of general formula (I) as defined supra.

The present invention provides the intermediate compounds which are disclosed in the Example Section of this text, infra.

The present invention provides any sub-combination within any embodiment or aspect of the present invention of intermediate compounds of general formulae (II), (III), (IV), and (V) supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action, which could not have been predicted. Compounds of the present invention have surprisingly been found to effectively inhibit PRMT5 and it is possible therefore that said compounds be used for the treatment or prophylaxis of disorders, preferably hyperproliferative disorders in humans and animals.

Compounds of the present invention can be utilized to inhibit PRMT5 and to block proliferation of cancer cells. block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

A disorder which is characterized by an increased cell proliferation and/or cell division may be named as a hyperproliferative disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

The compounds of formula (I) or its pharmaceutical composition can be used in combination with chemotherapeutic agents and/or anti-cancer agents.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemo-therapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein to a cell is administered one or more compounds of the present invention in combination with conventional radiation therapy.

In accordance with a further aspect, the present invention provides compounds of general formula (I), as described supra, or stereoisomers, tautomers, hydrates, solvates, and salts thereof, particularly pharmaceutically acceptable salts thereof, or mixtures of same, for use in the treatment or prophylaxis of disorders, in particular hyperproliferative disorders, particularly cancer disorders.

The pharmaceutical activity of the compounds according to the invention can be explained by their activity as described in the introductory section above.

In accordance with a further aspect, the present invention provides compounds of general formula (I), as described supra, or stereoisomers, tautomers, hydrates, solvates, and salts thereof, particularly pharmaceutically acceptable salts thereof, or mixtures of same, for use in the treatment or prophylaxis of a disorder, in particular hyperproliferative disorders, particularly cancer disorders.

In accordance with a further aspect, the present invention provides the use of compounds of general formula (I), as described supra, or stereoisomers, tautomers, hydrates, solvates, and salts thereof, particularly pharmaceutically acceptable salts thereof, or mixtures of same, for the treatment or prophylaxis of disorders, in particular hyperproliferative disorders, particularly cancer disorders.

In accordance with a further aspect, the present invention provides the use of a compound of formula (I), described supra, or a stereoisomer, a tautomer, a hydrate, a solvate, or a salt thereof, particularly a pharmaceutically acceptable salt thereof, or a mixture of same, for the treatment of disorders, in particular hyperproliferative disorders, particularly cancer disorders.

In accordance with a further aspect, the present invention provides the use of compounds of general formula (I), as described supra, or stereoisomers, tautomers, hydrates, solvates, and salts thereof, particularly pharmaceutically acceptable salts thereof, or mixtures of same, in a method of treatment or prophylaxis of disorders, in particular hyperproliferative disorders, particularly cancer disorders.

In accordance with a further aspect, the present invention provides the use of compounds of general formula (I), as described supra, or stereoisomers, tautomers, hydrates, solvates, and salts thereof, particularly pharmaceutically acceptable salts thereof, or mixtures of same, for the preparation of a medicament for the treatment or prophylaxis of a disorder, in particular hyperproliferative disorders, particularly cancer disorders.

In accordance with a further aspect, the present invention provides use of a compound of general formula (I), as described supra, or stereoisomers, tautomers, hydrates, solvates, and salts thereof, particularly pharmaceutically acceptable salts thereof, or mixtures of same, for the preparation of a pharmaceutical composition, preferably a medicament, for the prophylaxis or treatment of disorders, in particular hyperproliferative disorders, particularly cancer disorders.

In accordance with a further aspect, the present invention provides a method of treatment or prophylaxis of disorders, in particular hyperproliferative disorders, particularly cancer disorders, using an effective amount of a compound of general formula (I), as described supra, or stereoisomers, tautomers, hydrates, solvates, and salts thereof, particularly pharmaceutically acceptable salts thereof, or mixtures of same.

In accordance with a further aspect, the present invention provides a method for controlling a cancer disease in humans and/or animals by administering an effective amount of at least one compound as defined in one of claims 1 to 6, or a composition comprising said compound.

In accordance with a further aspect, the present invention provides pharmaceutical compositions, in particular a medicament, comprising a compound of general formula (I), as described supra, or a stereoisomer, a tautomer, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore provides pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides, fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention provides pharmaceutical combinations, in particular medicaments, comprising at least one compound of general formula (I) of the present invention and at least one or more further active ingredients, in particular for the treatment and/or prophylaxis of a hyperproliferative disorder, more particularly for treatment and/or prophylaxis of cancer disorders.

Particularly, the present invention provides a pharmaceutical combination, which comprises:
one or more first active ingredients, in particular compounds of general formula (I) as defined supra, and
one or more further active ingredients, chemotherapeutic agents and/or anti-cancer agents, in particular hyperproliferative disorders, more particularly for treatment and/or prophylaxis of cancer disorders.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also provides such pharmaceutical combinations. For example, the compounds of the present invention can be combined with known chemotherapeutic agents and/or anti-cancer agents.

Examples of chemotherapeutic agents and/or anti-cancer agents include:

131I-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib , crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, rucaparib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporf in, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

General Remarks:

In the text, the chemical names and the numbers of the compounds are given in bold. The intermediates are defined by INT-.

Chemical names were generated using the ACD/Name software from ACD/Labs. In some cases generally accepted names of commercially available reagents were used in place of ACD/Name generated names.

If the purity of the obtained example product are not mentioned, the compounds are 90 to 100% pure.

The following table 1 lists the abbreviations used in this paragraph and in the Examples section as far as they are not explained within the text body. Other abbreviations have their meanings customary per se to the skilled person.

TABLE 1
Abbreviations
The following table lists the abbreviations used herein.

| Abbreviation | Meaning |
|---|---|
| aq. | aqueous |
| cat. | catalytic |
| conc. | concentrated |
| DAD | diode array detector |
| DCM | dichloromethane |
| DIPEA | diisopropylethylamine |
| DMF | N,N-dimethylformamide |
| DMSO | dimethylsulfoxide |
| EtOAc | ethyl acetate |
| EtOH | ethanol |
| eq | equivalent |
| ESI | electrospray (ES) ionisation |
| h | hour(s) |
| HATU | 1-[bis(dimethylamino)methylene]-1H-1,2,3-triazolo[4,5- |
| | b]pyridinium 3-oxid hexafluorophosphate |
| HCl | hydrochloric acid |
| HPLC | high performance liquid chromatography |
| LC-MS | liquid chromatography mass spectrometry |
| min | minute(s) |
| MeCN | acetonitrile |
| MeOH | methanol |
| MS | mass spectrometry |
| NMR | nuclear magnetic resonance spectroscopy: chemical |
| | shifts (δ) are given in ppm. The chemical shifts were |
| | corrected by setting the DMSO signal to 2.50 ppm |
| | unless otherwise stated. |
| PDA | Photo Diode Array |
| Pd/C | palladium on activated charcoal |
| RT | room temperature |
| rac | racemic |
| Rt | retention time (as measured either with HPLC or UPLC) |
| | in minutes |
| sat. | saturated |
| SQD | Single-Quadrupole-Detector |
| t | triplet |
| T3P | propylphosphonic anhydride |
| TEA | triethylamine |
| TFA | trifluoroacetic acid |
| THF | tetrahydrofuran |
| UPLC | ultra performance liquid chromatography |

Other abbreviations have their meanings customary per se to the skilled person.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight” <1%.

The various aspects of the invention described in this application are illustrated by the following examples which are not meant to limit the invention in any way.

The example testing experiments described herein serve to illustrate the present invention and the invention is not limited to the examples given.

### EXPERIMENTAL SECTION

### General Part

All reagents, for which the synthesis is not described in the experimental part, are either commercially available, or are known compounds or may be formed from known compounds by known methods by a person skilled in the art.

Solvents

The following solvents were purchased from commercial sources and used without further purification:

acetic acid (CAS: 64-19-7)

acetonitrile (CAS: 75-05-8)

aqueous ammonia, 25% (CAS: 1336-21-6)

chloroform (CAS: 67-66-3)

dichloromethane (CAS: 75-09-2)

dimethyl sulfoxide (CAS: 67-68-5)

1,2-dimethoxyethane (CAS: 110-71-4)

ethanol (CAS: 64-17-5)

formic acid (CAS: 64-18-6)

methanol (CAS: 67-56-1)

N,N-dimethylformamide (CAS: 68-12-2)

tetrahydrofuran (CAS: 109-99-9)

toluene (CAS: 108-88-3)

pyridin (CAS: 110-86-1)

Reagents

All reagents, for which the synthesis is not described in the experimental part, are either commercially available or synthesized according to literature procedures.

The following reactants were purchased from commercial sources and were used without further purification:

N,N-diisopropylethylamine (CAS: 7087-68-5)

1-[bis(Dimethylamin)methylen]-1H-1,2,3-triazol[4,5-b]pyridinium-3-oxid-hexafluorophosphat (HATU) (CAS: 148893-10-1)

potassium iodide (CAS: 7681-11-0)

cesium carbonate (CAS: 534-17-8)

(4-methylphenyl)boronic acid (CAS: 5720-05-8)

sodium carbonate (CAS: 497-19-8)

tetrakis(triphenylphosphin)palladium(0) (CAS: 14221-01-3)

4,4,5,5-tetramethyl-2-phenyl-1,3,2-dioxaborolane (CAS: 24388-23-6)

sodium hydride (60% in mineral oil) (CAS: 7646-69-7)

propylphosphonic anhydride solution (T3P) (50% in DMF) (CAS: 68957-94-8)

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

Chromatographic Methods

Preparative column chromatographies, in particularly flash column chromatographies were performed using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or dichloromethane/methanol were used.

Preparative HPLC (HT) were performed on a Waters Autopurification MS SingleQuad instrument using a Waters XBridge C_{18 5} μm 100×30 mm column (flow: 70 mL/min; temperature: 25° C.; DAD scan: 210-400 nm) under eit her acidic conditions [eluent A: water+0.1 vol % formic acid (99%), eluent B: acetonitrile; gradient: 0-5.5 min 5-100% B] or a basic [eluent A: water+0.2 vol % aqueous ammonia, eluent B: acetonitrile; gradient: 0-5.5 min 5-100% B]. Before injection, the crude products were dissolved in a solvent (most of the time DMSO, THF or dichloromethane) and then filtered.

Analytical LC-MS/UPLCMS methods were either performed from UPLCMS on a Waters Acquity UPLCMS SingleQuad instrument (using a Acquity UPLC BEH C₁₈ 1.7 μm, 50×2.1 mm column or a Kinetex 2.6 μm 50×2.1 mm column) or on a Agilent 1290 UPLCMS 6230 TOF instrument using a BEH C₁₈ 1.7 μm, 50×2.1 mm column. The different methods used are described below:

Method 1:

Instrument: Waters Acquity UPLCMS SingleQuad; column: Acquity UPLC BEH C₁₈ 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

Method 2:

Instrument: Waters Acquity UPLCMS SingleQuad; column: Acquity UPLC BEH C₁₈ 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

High Throughput Methods:

Method 3:

Instrument: Waters Acquity UPLCMS SingleQuad; column: Acquity UPLC BEH C₁₈ 1.7 μm 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

Method 4:

Instrument: Waters Acquity UPLCMS SingleQuad; column: Acquity UPLC BEH C₁₈ 1.7 μm 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

Method 5:

Instrument: Waters Acquity with PDA detector and ZQ mass spectrometer; column: Acquity BEH C₁₈ 1.7 μm 2.1×50 mm; solvent A: Water+0.1% formic Acid; Solvent B: acetonitrile; gradient: 99% A to 1% A (1.6 min) to 1% A (0.4 min); flow: 0.8 mL/min; temperature: 60° C.; Injection Volume: 1.0 μL (0.1 m g-1 mg/mL sample concentration); Detection: PDA Scan Region 210-400 nm—plus fixed wavelength 254 nm; MS ESI (+), Scan region 170-800 m/z.

### EXPERIMENTAL SECTION

### General Procedures

General procedures (GP):

GP1: Amide Coupling Using HATU as a Coupling Agent

The amine partner (1.0 eq.), the acid partner (1.0 eq) and the commercially available coupling agent HATU (1.1 eq.) were dissolved in DMF (0.4 mL/mmol) under argon atmosphere. After degassing with argon for 5 min, DIPEA (1.1-3.0 eq.) was added and the mixture was stirred at RT for 16 h-3 d. After reaction completion, the mixture was diluted with EtOAc, water was added and the layers were separated. The aqueous layer was extracted with EtOAc. The combined organic layers were washed with brine and dried with sodium sulfate. After filtration, the solvent was removed under vacuum. The residue was purified by HPLC HT or column chromatography to give the desired amide.

GP2: SN Reaction Using Sodium Hydride

According to literature: J. Med. Chem., 2006, 49, 6819-6832, the aryl bromide partner (1 eq.) and the nucleophile (4 eq.) were dissolved in THF (4.6 mL/mmol) under argon atmosphere. After degassing the solution with argon for 5 min, sodium hydride (60% in mineral oil, 8 eq.) was carefully added (portionwise). The resulting mixture was stirred at RT for 30 min and then further stirred at 67-70° C. overnight. In cases where the reaction was not completed, additional sodium hydride (60% in mineral oil, 8 eq.) was added and the mixture was further heated to 70° C. overnight. After cooling, two different methods were used for work up:

Method A: MeOH (11.5 mL/mmol) and very little amount of acetic acid were added and the mixture was concentrated under vacuum. Water (15 mL/mmol), MeOH (6 mL/mmol) and saturated aqueous sodium hydrogencarbonate (3 mL/mmol) were added to the resulted residue. The suspension was finally stirred for 15 min, and the resulted solid was filtered off, washed with water and dried under vacuum.

Method B (mostly for example compounds 34-53): The mixture was evaporated in vaccuo. Then the resulted residue was redissolved in DMSO and filtered. The filter was washed with ACN/water (9:1) and the combined filtrates were evaporated in vaccuo.

The resulted product was either directly submitted for profiling without any further purification or purified by HPLC HT.

### EXPERIMENTAL SECTION

### Intermediates

Intermediate INT-1:

### N-(5-Bromo-1H-pyrazol-3-yl)-2-[(4-chlorophenyl)amino]-1,3-thiazole-4-carboxamide

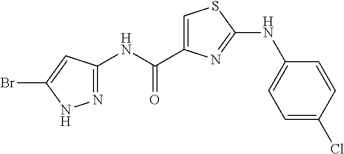 [SMILES: O=C(Nc1cc(Br)[nH]n1)c1csc(Nc2ccc(Cl)cc2)n1]

According to GP1, commercially available 5-bromo-1H-pyrazol-3-amine (CAS: 1203705-55-8, 950739-21-6, 385 mg, 2.38 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 550 mg, 2.16 mmol), HATU (903 mg, 2.38 mmol) and DIPEA (410 μL, 2.40 mmol) were stirred in DMF (4.2 mL) for 17 h. After reaction completion a colourless suspension was obtained. For work up, the reaction mixture was poured into water (50 mL), then the precipitate was filtered off and washed with water. The obtained solid was dried under vacuum giving INT-1 (795 mg, 76% yield) as a colourless, which was used in the next step without any further purification.

¹H-NMR (400 MHz, DMSO-d₆) δ [ppm]: 1.293 (0.52), 2.318 (0.60), 2.518 (6.63), 2.523 (4.84), 2.678 (0.60), 2.686 (1.95), 2.729 (1.23), 2.888 (1.51), 2.932 (0.52), 3.318 (1.75), 3.407 (0.40), 5.520 (1.51), 6.348 (1.19), 7.058 (0.48), 7.302 (0.79), 7.346 (1.39), 7.354 (14.41), 7.359 (4.33), 7.371 (5.00), 7.376 (16.00), 7.385 (1.71), 7.390 (1.11), 7.629 (0.52), 7.652 (0.44), 7.734 (1.07), 7.739 (0.56), 7.757 (2.34), 7.766 (9.81), 7.788 (10.32), 7.805 (0.75), 8.311 (1.35), 10.477 (1.11), 10.547 (5.52), 12.867 (1.07); LC-MS (method 2): Rₜ=1.13 min; MS (ESIpos): m/z=398 [M+H]⁺.

Intermediate INT-2:

### 2-Bromo-N-(5-tert-butyl-1,2-oxazol-3-yl)-1,3-thiazole-4-carboxamide

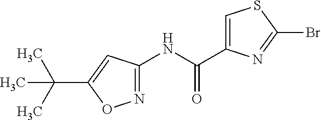 [SMILES: CC(C)(C)c1cc(NC(=O)c2csc(Br)n2)no1]

According to GP1, commercially available 2-bromo-1,3-thiazole-4-carboxylic acid (CAS: 5198-88-9, 816 mg, 3.92 mmol), commercially available 5-tert-butyl-1,2-oxazol-3-amine (CAS: 55809-36-4, 500 mg, 3.57 mmol), HATU (1.49 g, 3.92 mmol) and DIPEA (680 μL, 3.90 mmol) were stirred in DMF (7 mL) for 29 h. The obtained crude material was purified by Biotage (column: KP-SIL-100 g, gradient: hexane/EtOAc: 20% to 50% EtOAc), giving the desired amide INT-2 (928 mg, 68% yield) as an orange solid. ¹H-NMR (400 MHz, DMSO-d₆) δ [ppm]: 1.313 (16.00), 2.518 (0.58), 6.634 (2.56), 8.554 (2.32), 11.113 (0.51); LC-MS (method 2): Rₜ=1.25 min; MS (ESIpos): m/z=330 [M+H]⁺.

Intermediate INT-3:

### 2-(2-Bromo-5-nitrophenoxy)-N,N-diethylethanamine

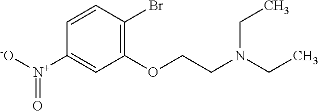

Commercially available 2-bromo-5-nitrophenol (CAS: 52427-05-1, 2.00 g, 9.17 mmol), commercially available 2-chloro-N,N-diethylethanamine hydrochloride (1:1) (CAS: 869-24-9, 1.89 g, 11.0 mmol), cesium carbonate (4.48 g, 13.8 mmol, 1.5 eq) and potassium iodide (305 mg, 1.83 mmol, 0.20 eq) were suspended in DMF (84 mL) under argon atmosphere. The resulted red reaction mixture was heated to 30° C. overnight. After reaction completion, the solvent was removed under vacuum. The resulted mixture was diluted with EtOAc and water was added. The layers were separated. The aqueous layer was extracted three times with EtOAc. The combined organic layers were washed with brine and dried with sodium sulfate. After filtration, the solvent was removed under vacuum giving the desired INT-3 (3.35 g) as an orange oil which was used in the next step without any further purification. ¹H-NMR (400 MHz, DMSO-d₆) δ [ppm]: 0.961 (7.25), 0.979 (16.00), 0.996 (7.44), 2.518 (0.52), 2.542 (2.18), 2.560 (6.82), 2.578 (6.66), 2.596 (1.98), 2.823 (1.60), 2.838 (3.52), 2.851 (1.68), 4.234 (1.65), 4.248 (3.42), 4.262 (1.59), 7.730 (1.24), 7.736 (1.30), 7.752 (1.55), 7.758 (1.68), 7.873 (2.42), 7.880 (5.25), 7.902 (2.52); LC-MS (method 2): Rₜ=1.40 min; MS (ESIpos): m/z=317 [M+H]⁺.

Intermediate INT-4:

### N,N-Diethyl-2-[(4′-methyl-4-nitrobiphenyl-2-yl)oxy]ethanamine

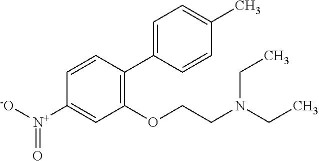

The aforementioned INT-4 (2.25 g, 94% purity, 6.67 mmol) was suspended in 1,2-dimethoxyethane (30 mL) under argon atmosphere. Then the commercially available (4-methylphenyl)boronic acid (1.18 g, 8.67 mmol), aqueous sodium carbonate solution (24 mL, 2 M, 47.8 mmol) and the commercially available catalyst tetrakis(triphenylphosphin)palladium(0) (293 mg, 0.253 mmol) were stirred overnight at 90° C. After cooling, the resulted mixture was diluted with dichloromethane and the layers were separated. The aqueous layer was extracted three times with dichloromethane. The combined organic layers were washed with brine and dried with sodium sulfate. After filtration, the solvent was removed under vacuum giving the desired product INT-4 (brown oil, 2.86 g, 75% purity, 98% yield) which was used in the next step without any further purification. ¹H-NMR (400 MHz, DMSO-d₆) δ [ppm]: 0.889 (7.30), 0.906 (16.00), 0.924 (7.58), 2.170 (1.54), 2.294 (2.74), 2.326 (1.61), 2.349 (10.87), 2.462 (1.33), 2.480 (3.68), 2.523 (0.73), 2.729 (0.96), 2.742 (1.60), 2.755 (0.91), 3.231 (3.54), 3.422 (2.34), 4.158 (1.49), 4.172 (2.94), 4.186 (1.41), 5.756 (1.85), 6.621 (0.48), 6.642 (0.56), 7.124 (0.71), 7.143 (0.74), 7.245 (2.86), 7.264 (3.23), 7.507 (4.29), 7.528 (3.83), 7.543 (0.57), 7.546 (0.69), 7.549 (0.72), 7.554 (3.26), 7.564 (1.29), 7.576 (3.34), 7.591 (0.51), 7.595 (0.89), 7.612 (0.74), 7.621 (0.85), 7.625 (0.96), 7.629 (0.51), 7.641 (0.54), 7.645 (0.51), 7.665 (0.94), 7.685 (0.85), 7.807 (0.41), 7.870 (0.96), 7.876 (2.78), 7.881 (3.04), 7.887 (2.75), 7.893 (2.38), 7.899 (0.92), 7.924 (2.03), 9.081 (0.48); LC-MS (method 2): Rₜ=1.59 min; MS (ESIpos): m/z=329 [M+H]⁺.

Intermediate INT-5:

### 2-[2-(Diethylamino)ethoxy]-4′-methylbiphenyl-4-amine

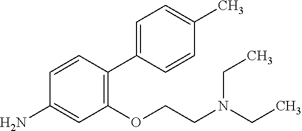 [SMILES: CCN(CC)CCOc1cc(N)ccc1-c1ccc(C)cc1]

The aforementioned INT-4 (666 mg, 75% purity, 1.52 mmol) was dissolved in ethanol (10 mL) under argon atmosphere. Then palladium on carbon (1.0 g, 5w%, 4.72 mmol) was added. The suspension was flushed with argon and vigorously shaken under an atmosphere of hydrogen (1 bar) for 4 h. For work up, the catalyst was removed by filtration and washed with ethanol. The filtrate was concentrated under vacuum giving INT-5 as a light brown oil, which was used in the next step without any further purification. ¹H-NMR (400 MHz, DMSO-d₆) δ [ppm]: 0.903 (7.36), 0.921 (16.00), 0.939 (7.65), 1.037 (4.03), 1.054 (9.15), 1.071 (4.34), 1.906 (0.42), 2.171 (2.36), 2.283 (11.71), 2.296 (5.21), 2.328 (0.60), 2.518 (1.90), 2.523 (1.23), 2.670 (0.53), 2.697 (1.18), 3.406 (0.43), 3.419 (0.52), 3.424 (1.26), 3.436 (1.29), 3.442 (1.25), 3.454 (1.25), 3.459 (0.47), 3.877 (1.34), 3.891 (2.65), 3.906 (1.30), 4.346 (0.64), 4.359 (1.21), 4.372 (0.61), 5.154 (3.20), 6.191 (1.43), 6.196 (1.57), 6.212 (1.46), 6.217 (1.65), 6.291 (2.89), 6.297 (2.64), 6.623 (0.71), 6.644 (0.84), 6.922 (3.46), 6.936 (0.74), 6.942 (3.37), 6.956 (0.56), 7.087 (3.04), 7.107 (3.60), 7.125 (1.30), 7.144 (1.34), 7.322 (4.52), 7.342 (3.72), 7.547 (0.89), 7.549 (0.86), 7.555 (0.74), 7.558 (0.66), 7.564 (1.03), 7.573 (0.86), 7.592 (0.71), 7.596 (1.31), 7.614 (1.09), 7.622 (1.17), 7.626 (1.49), 7.631 (0.74), 7.643 (0.75), 7.666 (1.63), 7.685 (1.47), 7.926 (4.28), 9.079 (0.75); LC-MS (method 2): Rₜ=1.36 min; MS (ESIpos): m/z=299 [M+H]⁺.

### EXPERIMENTAL SECTION

### EXAMPLES

### Example 1

### 2-[(4-Chlorophenyl)amino]-N-(5-phenyl-1H-pyrazol-3-yl)-1,3-thiazole-4-carboxamide

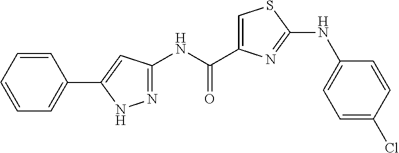 [SMILES: O=C(Nc1cc(-c2ccccc2)[nH]n1)c1csc(Nc2ccc(Cl)cc2)n1]

INT-1 (60.0 mg, 0.14 mmol) was suspended in 1,2-dimethoxyethane (0.6 mL) under argon atmosphere. Then tetrakis(triphenylphosphin)palladium(0) (15.7 mg, 0.014 mmol), aqueous sodium carbonate solution (320 μL, 2 M, 0.64 mmol) and 4,4,5,5-tetramethyl-2-phenyl-1,3,2-dioxaborolane (30.4 mg, 0.150 mmol) were added. The resulted light yellow mixture was heated to 115° C. for 20 h. To the suspension was additional tetrakis (triphenylphosphin)palladium(0) (0.5 eq) added and the mixture was further heated at 115° C. for 1 d. The reaction mixture was finally evaporated, DMSO was added and the solution was filtered (Sigma-Aldrich, Chemrus disposable filter funnel, 0.10 μm medium frit). The resulted residue was purified using preparative HPLC (method 3), giving the desired coupling product 1 (2.30 mg, 80% purity, 3% yield). ¹H-NMR (400 MHz, DMSO-d₆) δ [ppm]: 1.066 (0.44), 1.292 (1.53), 2.171 (6.11), 2.336 (1.45), 2.428 (1.31), 2.439 (0.58), 2.518 (16.00), 2.523 (11.13), 2.540 (1.67), 2.673 (3.05), 2.678 (1.38), 3.181 (0.44), 3.194 (0.44), 5.677 (0.51), 6.965 (1.02), 7.238 (0.44), 7.339 (1.60), 7.357 (3.35), 7.377 (11.71), 7.382 (4.07), 7.393 (4.22), 7.399 (11.27), 7.407 (1.89), 7.445 (3.93), 7.464 (6.18), 7.483 (3.13), 7.529 (0.73), 7.546 (0.73), 7.573 (0.51), 7.595 (0.58), 7.613 (0.51), 7.622 (0.58), 7.641 (0.51), 7.677 (0.58), 7.710 (0.73), 7.748 (9.60), 7.759 (8.73), 7.762 (9.96), 7.776 (7.05), 7.780 (7.13), 7.785 (6.69), 8.563 (0.58), 10.552 (0.51); LC-MS (method 2): Rₜ=1.21 min; MS (ESIneg): m/z=394 [M−H]⁻.

### Example 2

### N-(5-tert-Butyl-1,2-oxazol-3-yl)-2-(pyridin-2-ylamino)-1,3-thiazole-4-carboxamide

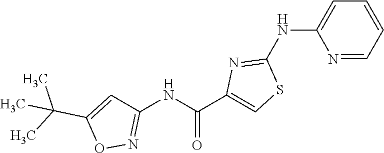 [SMILES: CC(C)(C)c1cc(NC(=O)c2csc(Nc3ccccn3)n2)no1]

According to GP1, commercially available 5-tert-butyl-1,2-oxazol-3-amine (CAS: 55809-36-4, 85.0 mg, 0.61 mmol), commercially available 2-(pyridin-2-ylamino)-1,3-thiazole-4-carboxylic acid (CAS: 232596-44-0, U.S. Pat. No. 6,100,282 A1, 2000, US2012/29029A1, 134 mg, 0.61 mmol), HATU (254 mg, 0.670 mmol) and DIPEA (0.12 mL, 0.67 mmol) were stirred in DMF (1.9 mL) overnight. After work up the crude material was purified using preparative HPLC (method 3) giving the desired amid 2 (50 mg, 22% yield) as a beige solid. ¹H-NMR (400 MHz, DMSO-d₆) δ [ppm]: 1.287 (0.98), 1.323 (16.00), 2.075 (0.86), 2.518 (0.75), 2.523 (0.49), 6.711 (2.65), 7.072 (0.50), 7.093 (0.51), 7.921 (2.04); LC-MS (method 2): Rₜ=1.29 min; MS (ESIneg): m/z=343 [M−H]⁻.

### Example 3

### N-(5-tert-Butyl-1,2-oxazol-3-yl)-2-(phenylamino)-1,3-thiazole-4-carboxamide

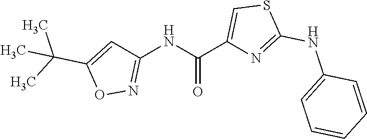 [SMILES: CC(C)(C)c1cc(NC(=O)c2csc(Nc3ccccc3)n2)no1]

According to GP1, commercially available 5-tert-butyl-1,2-oxazol-3-amine (CAS: 55809-36-4, 100 mg, 0.71 mmol), commercially available 2-(phenylamino)-1,3-thiazole-4-carboxylic acid (CAS: 165683-01-2, WO2011/109059A1, 150 mg, 95% purity, 0.65 mmol), HATU (271 mg, 0.71 mmol) and DIPEA (120 μL, 0.71 mmol) were stirred in DMF (1.6 mL). After workup, the crude product was purified using preparative HPLC (method 3) giving the desired amid 3 (121 mg, 47% yield) as a colorless solid. ¹H-NMR (400 MHz, DMSO-d₆) δ [ppm]: 1.324 (16.00), 6.696 (2.56), 6.995 (0.48), 7.321 (0.63), 7.340 (0.69), 7.343 (0.79), 7.361 (0.63), 7.716 (0.71), 7.719 (0.84), 7.738 (0.77), 7.741 (0.63), 7.792 (2.48); LC-MS (method 2): Rₜ=1.34 min; MS (ESIpos): m/z=343 [M+H]⁺.

### Example 4

### N-(5-tert-Butyl-1,2-oxazol-3-yl)-2-[(4-chlorophenyl)amino]-1,3-thiazole-4-carboxamide

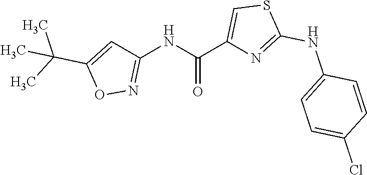 [SMILES: CC(C)(C)c1cc(NC(=O)c2csc(Nc3ccc(Cl)cc3)n2)no1]

According to GP1, commercially available 5-tert-butyl-1,2-oxazol-3-amine (CAS: 55809-36-4, 100 mg, 0.71 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 174 mg, 95% purity, 0.65 mmol), HATU (271 mg, 0.71 mmol) and DIPEA (120 μL, 0.71 mmol) were stirred in DMF (1.6 mL). After workup, the crude product was purified using preparative HPLC (method 3) giving the desired amide 4 (146 mg, 52% yield) as a colorless solid. ¹H-NMR (400 MHz, DMSO-d₆) δ [ppm]: 1.324 (16.00), 2.518 (0.44), 6.692 (2.97), 7.351 (1.28), 7.374 (1.47), 7.777 (1.36), 7.799 (1.17), 7.819 (2.51); LC-MS (method 2): Rₜ=1.43 min; MS (ESIpos): m/z=377 [M+H]⁺.

### Example 5

### 2-[(4-Chlorophenyl)amino]-N-(3-cyclopropyl-1H-pyrazol-5-yl)-1,3-thiazole-4-carboxamide

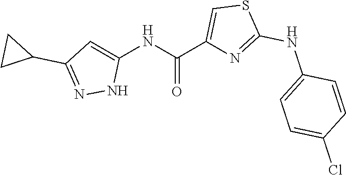 [SMILES: O=C(Nc1cc(C2CC2)n[nH]1)c1csc(Nc2ccc(Cl)cc2)n1]

According to GP1, commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 188 mg, 0.74 mmol), 3-cyclopropyl-1H-pyrazol-5-amine (CAS: 175137-46-9, 100 mg, 0.81 mmol), HATU (309 mg, 0.81 mmol) and DIPEA (0.14 mL, 0.81 mmol) were stirred in DMF (1.3 mL) for 21 h. After workup, the crude product was purified using preparative HPLC (method 3) giving the desired amide 5 (15 mg, 5% yield) as a light brown solid. ¹H-NMR (400 MHz, DMSO-d₆) δ [ppm]: 0.676 (1.93), 0.688 (6.07), 0.692 (5.87), 0.700 (6.16), 0.715 (1.96), 0.850 (0.56), 0.913 (2.04), 0.922 (4.89), 0.928 (4.86), 0.943 (5.19), 0.948 (4.62), 0.960 (1.51), 1.231 (0.92), 1.865 (0.92), 1.878 (1.66), 1.887 (1.87), 1.900 (2.84), 1.912 (1.69), 1.921 (1.42), 1.933 (0.68), 2.074 (7.05), 2.336 (0.59), 2.518 (8.39), 2.522 (5.60), 2.539 (0.95), 2.678 (0.62), 2.686 (1.04), 6.263 (5.72), 7.369 (13.87), 7.374 (4.83), 7.386 (5.10), 7.392 (16.00), 7.687 (11.91), 7.715 (9.57), 7.738 (8.65), 9.695 (6.49), 10.520 (9.04), 12.231 (4.77); LC-MS (method 2): Rₜ=1.12 min; MS (ESIpos): m/z=360 [M+H]⁺

### Example 6

### N-(5-tert-Butyl-1,2-oxazol-3-yl)-2-[(5-fluoropyridin-2-yl)amino]-1,3-thiazole-4-carboxamide

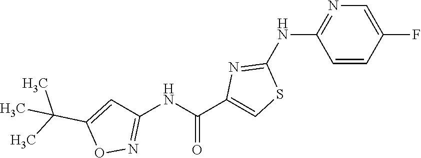 [SMILES: CC(C)(C)c1cc(NC(=O)c2csc(Nc3ccc(F)cn3)n2)no1]

According to GP2, INT-5 (60 mg, 0.17 mmol) and commercially available 5-fluoropyridin-2-amine (CAS: 21717-96-4, 77.4 mg, 0.69 mmol) sodium hydride (55.2 mg, 60% in mineral oil, 1.38 mmol) were stirred in THF (0.8 mL) under argon atmosphere. After degassing for 5 min with argon, was carefully added (portionwise). After workup, the resulted solid was washed with water and dried in vacuo, giving the desired product 6 (61.1 mg , 88% yield) as a beige solid. ¹H-NMR (400 MHz, DMSO-d₆) δ [ppm]: 1.321 (16.00), 2.518 (1.02), 2.523 (0.68), 6.706 (2.55), 7.908 (1.89), 8.331 (0.68), 8.339 (0.67); LC-MS (method 2): Rₜ=1.32 min; MS (ESIpos): m/z=362 [M+H]⁺.

### Example 7

### N-(5-tert-Butyl-1,2-oxazol-3-yl)-2-[(4-fluorophenyl)amino]-1,3-thiazole-4-carboxamide

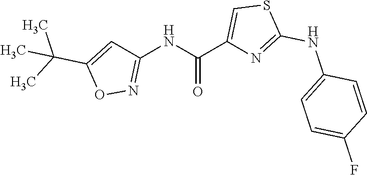 [SMILES: CC(C)(C)c1cc(NC(=O)c2csc(Nc3ccc(F)cc3)n2)no1]

According to GP1, commercially available 5-tert-butyl-1,2-oxazol-3-amine (CAS: 55809-36-4, 100 mg, 0.71 mmol), commercially available 2-[(4-fluorophenyl)amino]-1,3-thiazole-4-carboxylic acid (CAS: 887405-91-6, WO2006/58905 A1, 154 mg, 0.65 mmol), HATU (271 mg, 0.71 mmol) and DIPEA (120 μL, 0.71 mmol) were stirred in DMF (1.6 mL). After workup, the crude product was purified using preparative HPLC (method 3) giving the desired amide 7 (117 mg, 43% yield) as a colorless solid. ¹H-NMR (400 MHz, DMSO-d₆) δ [ppm]: 1.323 (16.00), 6.691 (2.61), 7.143 (0.59), 7.166 (0.93), 7.187 (0.61), 7.757 (0.55), 7.769 (0.59), 7.780 (0.67), 7.784 (2.90), 7.792 (0.57); LC-MS (method 2): Rₜ=1.35 min; MS (ESIpos): m/z=361 [M+H]⁺.

### Example 8

### N-(1,3-Benzothiazol-2-yl)-2-[(4-chlorophenyl)amino]-1,3-thiazole-4-carboxamide

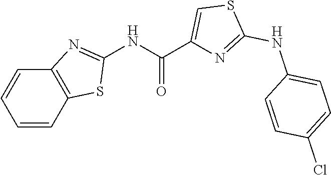 [SMILES: O=C(Nc1nc2ccccc2s1)c1csc(Nc2ccc(Cl)cc2)n1]

According to GP1, commercially available 1,3-benzothiazol-2-amine (CAS: 136-95-8, 32.4 mg, 0.22 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 50.0 mg, 0.2 mmol), HATU (82.1 mg, 0.22 mmol) and DIPEA (50 μL, 0.29 mmol) were stirred in DMF (0.35 mL) for 3 d. After workup, the crude product was purified using preparative HPLC (method 3) giving the desired amide 8 (14 mg, 15% yield) as a colorless solid. ¹H-NMR (400 MHz, DMSO-d₆) δ [ppm]: 1.229 (0.41), 2.518 (5.65), 2.523 (4.20), 7.327 (2.62), 7.329 (2.80), 7.347 (4.88), 7.364 (3.47), 7.367 (3.66), 7.383 (1.24), 7.391 (15.30), 7.397 (3.97), 7.408 (4.41), 7.413 (15.97), 7.421 (1.43), 7.456 (3.37), 7.459 (3.32), 7.475 (3.29), 7.477 (4.85), 7.480 (4.15), 7.495 (2.96), 7.498 (2.85), 7.796 (4.41), 7.816 (3.92), 7.839 (1.56), 7.847 (15.35), 7.853 (4.38), 7.864 (4.15), 7.870 (14.11), 7.878 (1.24), 8.024 (4.95), 8.035 (16.00), 8.042 (4.88), 10.589 (5.91), 12.222 (0.99); LC-MS (method 2): Rₜ=1.28 min; MS (ESIpos): m/z=387 [M+H]⁺.

### Example 9

### 2-[(4-Chlorophenyl)amino]-N-(5-methoxy-1H-benzimidazol-2-yl)-1,3-thiazole-4-carboxamide

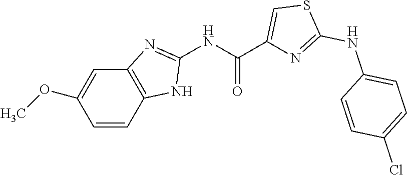 [SMILES: COc1ccc2[nH]c(NC(=O)c3csc(Nc4ccc(Cl)cc4)n3)nc2c1]

According to GP1, commercially available 5-methoxy-1H-benzimidazol-2-amine (CAS: 6232-91-3, 85.0 mg, 0.52 mmol), 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 133 mg, 0.52 mmol), HATU (218 mg, 0.57 mmol) and DIPEA (0.1 mL, 0.57 mmol) were stirred overnight. Then additional DIPEA (181 μL, 2 eq.) and HATU (218 mg, 0.57 mmol, 1 eq.) were added to the reaction mixture and the mixture was stirred for further 24 h at RT. After workup, the crude product was purified using preparative HPLC (method 3) and additional preparative HPLC (see method below) giving the desired amide 9 (10.2 mg, 80% purity, 4% yield) as a yellow solid. ¹H-NMR (400 MHz, DMSO-d₆) δ [ppm]: 0.851 (0.60), 1.230 (3.19), 1.256 (4.15), 1.295 (1.47), 1.331 (2.20), 2.518 (2.35), 2.523 (1.62), 3.768 (16.00), 6.744 (1.18), 6.751 (1.18), 6.766 (1.17), 6.772 (1.28), 7.034 (0.58), 7.353 (1.21), 7.375 (1.29), 7.381 (3.55), 7.386 (1.10), 7.398 (1.02), 7.404 (3.77), 7.810 (3.30), 7.815 (1.02), 7.827 (0.89), 7.832 (3.02), 7.913 (1.21), 10.569 (1.13); LC-MS (method 2): Rₜ=1.20 min; MS (ESIpos): m/z=400 [M+H]⁺.

Preparative HPLC method: Instrument: Waters Acquity UPLCMS SingleQuad; column: Acquity UPLC BEH C₁₈ 1.7 μm, 50×2.1 mm; eluent A: water+0.2 vol % aqueous ammoniac (32%); eluent B: acetonitrile; gradient: 0-1.6 min 1-99% B, 1.6-2.0 min 99% B; flow: 0.8 mL/min; temperature: 60° C.; DAD scan: 210-400 nm.

Preparation: Instrument: Waters Autopurificationsystem; column: Waters XBrigde C₁₈ 5.0 μm 100×30 mm; eluent A: water+0.2 vol % aqueous ammoniac (32%); eluent B: acetonitrile; gradient: 0.00-0.50 min 22% B (40 to 70 mL/min), 0.51-5.50 min 43-53% B (70 mL/min), DAD scan: 210-400 nm.

### Example 10

### N-[1-Methyl-5-(trifluoromethyl)-1H-pyrazol-3-yl]-2-(phenylamino)-1,3-thiazole-4-carboxamide

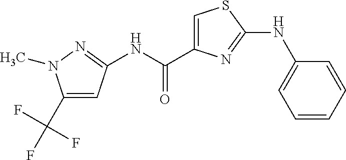 [SMILES: Cn1nc(NC(=O)c2csc(Nc3ccccc3)n2)cc1C(F)(F)F]

According to GP1, commercially available 2-(phenylamino)-1,3-thiazole-4-carboxylic acid (CAS: 165683-01-2, WO2011/109059A1, 90.0 mg, 0.41 mmol), commercially available 1-methyl-5-(trifluoromethyl)-1H-pyrazol-3-amine (CAS: 149978-42-7, 81.0 mg, 0.49 mmol), HATU (233 mg, 0.61 mmol) and DIPEA (0.12 mL, 0.67 mmol) were stirred in DMF (1.9 mL) overnight. After workup, the crude product was purified using preparative HPLC (method 4) giving the desired amide 10 (70 mg, 47% yield). ¹H-NMR (400 MHz, DMSO-d₆) δ [ppm]: 2.074 (0.80), 2.518 (2.05), 2.522 (1.36), 3.928 (16.00), 6.975 (0.92), 6.978 (1.51), 6.980 (0.86), 6.996 (3.18), 7.015 (1.79), 7.105 (7.09), 7.327 (3.76), 7.331 (1.25), 7.346 (4.52), 7.348 (4.85), 7.362 (1.23), 7.367 (3.65), 7.715 (4.74), 7.717 (5.30), 7.736 (5.79), 7.740 (15.24), 10.286 (4.92), 10.391 (4.69); LC-MS (method 1): Rₜ=1.25 min; MS (ESIpos): m/z=368 [M+H]⁺.

### Example 11

### 2-[(4-Chlorophenyl)amino]-N-(1,3-thiazol-2-yl)-1,3-thiazole-4-carboxamide

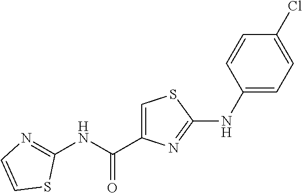 [SMILES: O=C(Nc1nccs1)c1csc(Nc2ccc(Cl)cc2)n1]

According to GP1, commercially available 1,3-thiazol-2-amine (CAS: 96-50-4, 120 mg, 1.20 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 305 mg, 1.20 mmol), HATU (501 mg, 1.32 mmol) and DIPEA (230 μL, 1.3 mmol) were stirred in DMF (3.8 mL) overnight. After workup, the crude product was purified using preparative HPLC (method 3) giving the desired amide 11 (175 mg, 41% yield) as a light brown solid. ¹H-NMR (400 MHz, DMSO-d₆) δ [ppm]: 2.074 (0.44), 2.518 (0.73), 2.523 (0.49), 7.296 (8.22), 7.305 (8.81), 7.361 (0.85), 7.369 (9.96), 7.374 (2.75), 7.386 (3.02), 7.391 (10.56), 7.399 (0.96), 7.564 (8.54), 7.573 (8.03), 7.806 (1.03), 7.814 (10.73), 7.820 (2.90), 7.832 (2.85), 7.837 (9.18), 7.845 (0.85), 7.937 (16.00), 10.571 (0.42); LC-MS (method 2): Rₜ=1.17 min; MS (ESIpos): m/z=337 [M+H]⁺.

### Example 12

### N-(5-tert-Butyl-1H-pyrazol-3-yl)-2-[(4-chlorophenyl)amino]-1,3-thiazole-4-carboxamide

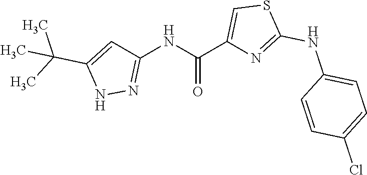 [SMILES: CC(C)(C)c1cc(NC(=O)c2csc(Nc3ccc(Cl)cc3)n2)n[nH]1]

According to GP1, commercially available, 5-tert-butyl-1H-pyrazol-3-amine (CAS: 82560-12-1, 30.1 mg, 0.22 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 50.0 mg, 0.2 mmol), HATU (82.1 mg, 0.22 mmol) and DIPEA (51 μL, 0.29 mmol) were stirred in DMF (0.35 mL) for 3 d. After workup, the crude product was purified using preparative HPLC (method 3) giving the desired amide 12 (20 mg, 23% yield) as a colourless solid. ¹H-NMR (400 MHz, DMSO-d₆) δ [ppm]: 1.280 (16.00), 6.377 (0.45), 7.362 (1.76), 7.367 (0.60), 7.379 (0.63), 7.384 (1.97), 7.694 (2.14), 7.722 (1.25), 7.744 (1.10), 10.529 (0.97), 12.215 (0.49); LC-MS (method 2): Rₜ=1.19 min; MS (ESIpos): m/z=375 [M+H]⁺.

### Example 13

### N-(5-tert-Butyl-1,2-oxazol-3-yl)-2-[(4-nitrophenyl)amino]-1,3-thiazole-4-carboxamide

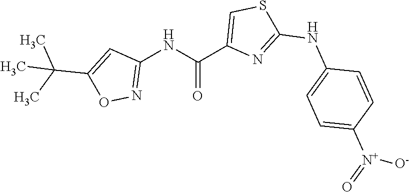 [SMILES: CC(C)(C)c1cc(NC(=O)c2csc(Nc3ccc([N+](=O)[O-])cc3)n2)no1]

According to GP1, commercially available 5-tert-butyl-1,2-oxazol-3-amine (CAS: 55809-36-4, 100 mg, 0.71 mmol), commercially available 2-[(4-nitrophenyl)amino]-1,3-thiazole-4-carboxylic acid (CAS: 886496-56-6, Eur. J. Med. Chem., 2016, 123, 718-726, 172 mg, 0.65 mmol), HATU (271 mg, 0.71 mmol) and DIPEA (120 μL, 0.71 mmol) were stirred in DMF (1.6 mL) overnight. After workup, the crude product was purified using preparative HPLC (method 3) giving the desired amide 13 (170 mg, 59% yield) as a yellow solid. ¹H-NMR (400 MHz, DMSO-d₆) δ [ppm]: 1.331 (16.00), 2.518 (0.49), 6.703 (2.49), 7.981 (2.66), 7.989 (1.06), 8.012 (1.28), 8.212 (1.59), 8.235 (1.27), 10.819 (0.55); LC-MS (method 2): Rₜ=1.31 min; MS (ESIpos): m/z=388 [M+H]⁺.

### Example 14

### N-(3-Cyclopropyl-1H-pyrazol-5-yl)-2-(phenylamino)-1,3-thiazole-4-carboxamide

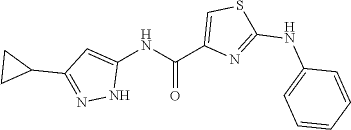 [SMILES: O=C(Nc1cc(C2CC2)n[nH]1)c1csc(Nc2ccccc2)n1]

According to GP1, commercially available 3-cyclopropyl-1H-pyrazol-5-amine (CAS: 175137-46-9, 36.9 mg, 0.3 mmol), commercially available 2-(phenylamino)-1,3-thiazole-4-carboxylic acid (CAS: 165683-01-2, WO2011/109059A1, 60.0 mg, 0.27 mmol), HATU (114 mg, 0.3 mmol) and DIPEA (52 μL, 0.3 mmol) were stirred in DMF (0.6 mL) overnight. After workup, the crude product was purified using preparative HPLC (method 3) giving the desired amide 14 (44.2 mg, 43% yield) as a light orange solid. ¹H-NMR (400 MHz, DMSO-d₆) δ [ppm]: 0.686 (4.02), 0.690 (3.98), 0.698 (4.18), 0.924 (2.97), 0.941 (3.12), 1.865 (0.59), 1.877 (1.13), 1.886 (1.29), 1.898 (1.99), 1.910 (1.21), 1.919 (1.01), 1.931 (0.47), 2.518 (2.77), 2.523 (1.95), 2.540 (0.74), 2.685 (1.01), 3.307 (0.43), 3.328 (1.48), 3.802 (1.72), 6.269 (2.11), 6.979 (1.60), 6.982 (2.77), 6.984 (1.60), 7.000 (5.66), 7.016 (1.87), 7.019 (3.20), 7.021 (1.83), 7.334 (6.44), 7.338 (2.15), 7.353 (7.41), 7.355 (8.35), 7.369 (1.99), 7.374 (6.40), 7.655 (16.00), 7.675 (4.76), 7.797 (0.59), 9.604 (1.76), 10.394 (7.49), 12.228 (2.19); LC-MS (method 2): Rₜ=1.03 min; MS (ESIpos): m/z=326 [M+H]⁺.

### Example 15

### 2-[(4-Chlorophenyl)amino]-N-(1-ethyl-1H-pyrazol-3-yl)-1,3-thiazole-4-carboxamide

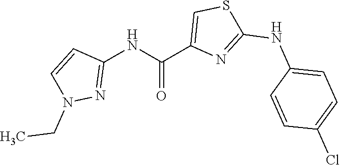 [SMILES: CCn1ccc(NC(=O)c2csc(Nc3ccc(Cl)cc3)n2)n1]

According to GP1, commercially available 1-ethyl-1H-pyrazol-3-amine (CAS: 55361-49-4, 50.0 mg, 0.45 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 115 mg, 0.45 mmol), HATU (188 mg, 0.5 mmol) and DIPEA (86 μL, 0.49 mmol) were stirred in DMF (1.0 mL) overnight. After workup, the crude product was purified using preparative HPLC (method 3) giving the desired amide 15 (78.2 mg, 47% yield) as a colourless solid. ¹H-NMR (400 MHz, DMSO-d₆) δ [ppm]: 1.354 (6.79), 1.372 (16.00), 1.390 (6.49), 2.075 (4.43), 2.518 (3.42), 2.523 (2.33), 4.047 (1.61), 4.065 (5.01), 4.083 (5.12), 4.102 (1.55), 6.545 (4.37), 6.551 (4.28), 7.361 (0.52), 7.369 (6.51), 7.374 (1.69), 7.386 (1.87), 7.391 (7.00), 7.399 (0.64), 7.673 (4.01), 7.679 (3.99), 7.710 (12.04), 7.739 (0.64), 7.747 (6.43), 7.752 (1.81), 7.764 (1.67), 7.770 (5.96), 7.778 (0.54), 9.946 (3.40), 10.509 (3.09); LC-MS (method 2): Rₜ=1.17 min; MS (ESIpos): m/z=348 [M+H]⁺.

### Example 16

### N-(5-tert-Butyl-1H-pyrazol-3-yl)-2-(phenylamino)-1,3-thiazole-4-carboxamide

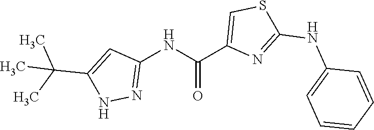 [SMILES: CC(C)(C)c1cc(NC(=O)c2csc(Nc3ccccc3)n2)n[nH]1]

According to GP1, commercially available 5-tert-butyl-1H-pyrazol-3-amine (CAS: 82560-12-1, 41.7 mg, 0.3 mmol), commercially available 2-(phenylamino)-1,3-thiazole-4-carboxylic acid (CAS: 165683-01-2, WO2011/109059A1, 60.0 mg, 0.27 mmol), HATU (114 mg, 0.3 mmol) and DIPEA (52 μL, 0.3 mmol) were stirred in DMF (660 μL) overnight. After workup, the crude product was purified using preparative HPLC (method 3) giving the desired amide 16 (57.1 mg, 53% yield) as a light orange solid; ¹H-NMR (400 MHz, DMSO-d₆) δ [ppm]: 1.283 (16.00), 2.518 (0.86), 2.522 (0.61), 2.685 (0.49), 6.380 (0.70), 6.984 (0.49), 7.003 (1.03), 7.021 (0.58), 7.336 (1.13), 7.340 (0.40), 7.354 (1.38), 7.357 (1.53), 7.376 (1.15), 7.655 (2.93), 7.674 (0.90), 9.620 (0.73), 10.398 (0.98), 12.208 (0.61); LC-MS (method 2): Rₜ=1.14 min; MS (ESIpos): m/z=342 [M+H]⁺.

### Example 17

### 2-[(4-Chlorophenyl)amino]-N-[4-(3,4-dihydroisoquinolin-2(1H)-yl)butyl]-1,3-thiazole-4-carboxamide

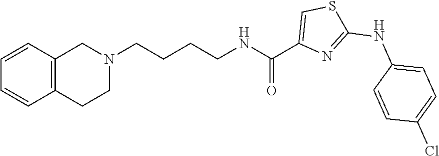 [SMILES: O=C(NCCCCN1CCc2ccccc2C1)c1csc(Nc2ccc(Cl)cc2)n1]

According to GP1, commercially available 4-(3,4-dihydroisoquinolin-2(1H)-yl)butan-1-amine dihydrochloride (CAS: 198895-32-8, WO9740051 A1 19971030, 120 mg, 0.43 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 100 mg, 0.39 mmol), HATU (164 mg, 0.43 mmol) and DIPEA (100 μL, 0.59 mmol) were stirred in DMF (700 μL) for 3 d. After workup, the crude product was purified using preparative HPLC (method 3) giving the desired amide 17 (16.0 mg, 8% yield) as a colourless solid. ¹H-NMR (400 MHz, DMSO-d₆) δ [ppm]: 1.407 (0.44), 1.560 (4.88), 1.570 (5.05), 1.576 (5.16), 2.323 (0.62), 2.327 (0.87), 2.332 (0.64), 2.447 (2.01), 2.463 (4.58), 2.480 (3.30), 2.518 (6.00), 2.523 (3.79), 2.611 (2.52), 2.626 (6.67), 2.640 (3.82), 2.665 (0.79), 2.669 (1.00), 2.673 (0.73), 2.768 (2.88), 2.782 (4.77), 2.796 (2.15), 3.275 (1.14), 3.291 (3.21), 3.306 (3.21), 3.518 (10.05), 7.002 (1.29), 7.008 (1.74), 7.020 (2.57), 7.047 (0.92), 7.060 (2.15), 7.071 (8.85), 7.076 (5.95), 7.084 (8.24), 7.093 (1.57), 7.098 (1.32), 7.311 (0.84), 7.319 (9.07), 7.324 (2.98), 7.336 (2.88), 7.341 (10.55), 7.349 (1.09), 7.489 (16.00), 7.731 (1.01), 7.739 (10.17), 7.744 (3.04), 7.756 (2.74), 7.761 (9.15), 7.770 (0.98), 8.103 (1.26), 8.118 (2.68), 8.133 (1.28), 10.436 (0.87); LC-MS (method 2): Rₜ=1.38 min; MS (ESIpos): m/z=441 [M+H]⁺.

### Example 18

### N-(5-tert-Butyl-1,2-oxazol-3-yl)-2-[(3-chloro-4-methylphenyl)amino]-1,3-thiazole-4-carboxamide

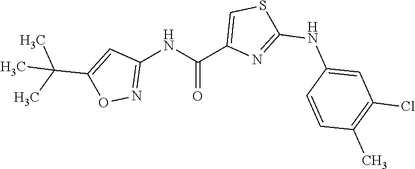 [SMILES: Cc1ccc(Nc2nc(C(=O)Nc3cc(C(C)(C)C)on3)cs2)cc1Cl]

According to GP1, commercially available 5-tert-butyl-1,2-oxazol-3-amine (CAS: 55809-36-4, 57.4 mg, 0.41 mmol), commercially available 2-[(3-chloro-4-methylphenyl)amino]-1,3-thiazole-4-carboxylic acid (CAS: 1176036-07-9, 100 mg, 0.37 mmol), HATU (156 mg, 0.41 mmol) and DIPEA (97 μL, 0.56 mmol) were stirred in DMF (660 μL) for 18 h. After workup, the crude product was purified using preparative HPLC (method 3) giving the desired amide 18 (3.00 mg, 90% purity, 2% yield) as a light browm solid. ¹H-NMR (400 MHz, DMSO-d₆) δ [ppm]: 1.324 (16.00), 2.266 (0.51), 2.283 (3.15), 2.518 (1.00), 2.523 (0.70), 6.703 (2.17), 7.289 (0.50), 7.310 (0.58), 7.596 (0.41), 7.602 (0.43), 7.771 (0.80), 7.777 (0.76), 7.833 (2.03), 10.477 (0.80), 10.655 (0.82); LC-MS (method 2): Rₜ=1.45 min; MS (ESIneg): m/z=390 [M−H]⁻.

### Example 19

### 2-[(4-Chlorophenyl)amino]-N-(1,2-oxazol-3-yl)-1,3-thiazole-4-carboxamide

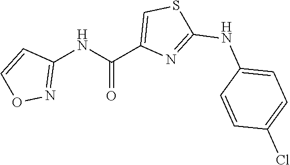 [SMILES: O=C(Nc1ccon1)c1csc(Nc2ccc(Cl)cc2)n1]

According to GP1, commercially available 1,2-oxazol-3-amine (CAS: 1750-42-1, 36.3 mg, 0.43 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 100 mg, 0.39 mmol), HATU (164 mg, 0.43 mmol) and DIPEA (100 μL, 0.59 mmol) were stirred in DMF (700 μL) for 3 d. After workup, the crude product was purified using preparative HPLC (method 3) giving the desired amide 19 (20.0 mg, 14% yield) as a colourless solid. ¹H-NMR (400 MHz, DMSO-d₆) δ [ppm]: 2.518 (1.21), 2.522 (0.84), 7.023 (8.57), 7.027 (8.65), 7.348 (0.70), 7.357 (8.64), 7.362 (2.25), 7.374 (2.56), 7.379 (9.02), 7.387 (0.79), 7.785 (0.86), 7.793 (9.01), 7.799 (2.46), 7.810 (2.39), 7.816 (8.13), 7.824 (0.71), 7.852 (16.00), 8.866 (8.58), 8.871 (8.63), 10.544 (1.95), 10.797 (1.75); LC-MS (method 2): Rₜ=1.15 min; MS (ESIpos): m/z=321 [M+H]⁺.

### Example 20

### 2-[(4-Chlorophenyl)amino]-N-(5-methyl-1,2-oxazol-3-yl)-1,3-thiazole-4-carboxamide

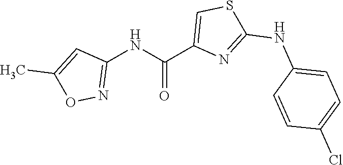 [SMILES: Cc1cc(NC(=O)c2csc(Nc3ccc(Cl)cc3)n2)no1]

According to GP1, commercially available 5-methyl-1,2-oxazol-3-amine (CAS: 1072-67-9, 21.2 mg, 0.22 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 50.0 mg, 0.2 mmol), HATU (82.1 mg, 0.22 mmol) and DIPEA (51 μL, 0.29 mmol) were stirred in DMF (350 μL) for 3 d. After workup, the crude product was purified using preparative HPLC (method 3) giving the desired amide 20 (4.00 mg, 90% purity, 5% yield) as a colourless solid. ¹H-NMR (400 MHz, CHLOROFORM-d) δ [ppm]: −0.008 (0.60), 0.008 (0.57), 0.070 (1.74), 1.124 (0.80), 1.457 (0.57), 1.631 (1.18), 2.013 (1.63), 2.441 (14.11), 2.893 (0.44), 2.966 (0.51), 3.919 (0.42), 6.816 (4.24), 6.818 (4.25), 7.298 (1.68), 7.324 (0.58), 7.331 (0.78), 7.339 (0.82), 7.356 (16.00), 7.370 (0.65), 7.379 (0.64), 7.598 (7.32), 9.429 (1.25); LC-MS (method 2): Rₜ=1.17 min; MS (ESIpos): m/z=335 [M+H]⁺.

### Example 21

### N-(5-tert-Butyl-1,2-oxazol-3-yl)-2-[(3,4-dichlorophenyl)amino]-1,3-thiazole-4-carboxamide

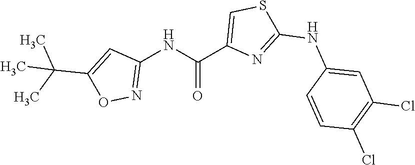 [SMILES: CC(C)(C)c1cc(NC(=O)c2csc(Nc3ccc(Cl)c(Cl)c3)n2)no1]

Commercially available 5-tert-butyl-1,2-oxazol-3-amine (CAS: 55809-36-4, 26.7 mg, 0.19 mmol) and 2-[(3,4-dichlorophenyl)amino]-1,3-thiazole-4-carboxylic acid (CAS: 952182-44-4, US2012/29029 A1, 50.0 mg, 0.17 mmol) were suspended in DMF (1.1 mL) under argon atmosphere. Then DIPEA (90 μL, 0.52 mmol) and commercially available T3P (200 μL, 50% in DMF, 0.35 mmol) were added. T3P was used here as HATU seemed not to be best mode for this case. The resulting yellow solution was stirred at RT over two nights. DMSO was added and the solution was filtered (Chromafil, O-45/15 MS, 0.45 μm, polytetrafluoroethylene). The obtained filtrate was purified using preparative HPLC (method 3) giving the desired amide 21 (8.80 mg, 11% yield) as a colourless solid. ¹H-NMR (400 MHz, DMSO-d₆) δ [ppm]: 1.324 (16.00), 2.518 (0.63), 2.523 (0.47), 6.702 (2.30), 7.540 (0.96), 7.562 (1.17), 7.732 (0.53), 7.738 (0.56), 7.753 (0.41), 7.760 (0.45), 7.887 (2.83), 8.019 (1.01), 8.026 (0.94); LC-MS (method 2): Rₜ=1.49 min; MS (ESIneg): m/z=409 [M−H]⁻.

### Example 22

### N-{2-[2-(Diethylamino)ethoxy]-4′-methylbiphenyl-4-yl}-2-(phenylamino)-1,3-thiazole-4-carboxamide

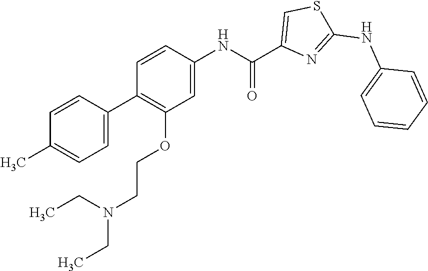 [SMILES: CCN(CC)CCOc1cc(NC(=O)c2csc(Nc3ccccc3)n2)ccc1-c1ccc(C)cc1]

According to GP1, INT-5 (85.2 mg, 0.29 mmol), 2-(phenylamino)-1,3-thiazole-4-carboxylic acid (CAS: 165683-01-2, WO2011/109059A1, 60.0 mg, 0.27 mmol), HATU (113 mg, 0.30 mmol) and DIPEA (52 μL, 0.30 mmol) were stirred in DMF (0.66 mL) at RT overnight and further 6 h at 40° C. After workup, the crude product was purified using preparative HPLC (method 3) giving the desired amide 22 (57.9 mg, 90% purity, 36% yield) as a ligh orange solid. ¹H-NMR (400 MHz, DMSO-d₆) δ [ppm]: 0.908 (6.98), 0.926 (16.00), 0.943 (7.27), 2.325 (7.94), 2.470 (2.08), 2.489 (7.92), 2.518 (0.72), 2.524 (2.09), 2.720 (1.27), 2.736 (2.99), 2.750 (1.33), 4.000 (1.28), 4.015 (2.67), 4.030 (1.22), 6.984 (0.44), 6.986 (0.73), 6.989 (0.45), 7.005 (1.51), 7.023 (0.84), 7.175 (2.22), 7.195 (2.56), 7.261 (2.34), 7.282 (2.65), 7.341 (1.75), 7.359 (2.16), 7.362 (2.37), 7.376 (0.60), 7.380 (1.71), 7.437 (3.62), 7.445 (1.63), 7.457 (2.92), 7.461 (1.61), 7.466 (1.21), 7.662 (1.99), 7.667 (1.92), 7.694 (7.27), 7.720 (2.26), 7.723 (2.61), 7.742 (2.38), 7.744 (1.89), 9.815 (2.49), 10.397 (1.61); LC-MS (method 2): Rₜ=1.62 min; MS (ESIneg): m/z=499.2 [M−H]⁻.

### Example 23

### N-(1-Methyl-1H-pyrazol-3-yl)-2-(phenylamino)-1,3-thiazole-4-carboxamide

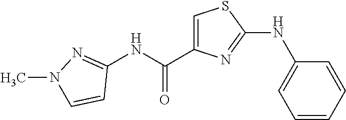 [SMILES: Cn1ccc(NC(=O)c2csc(Nc3ccccc3)n2)n1]

According to GP1, commercially available 2-(phenylamino)-1,3-thiazole-4-carboxylic acid (CAS: 165683-01-2, WO2011/109059A1, 90.0 mg, 0.41 mmol), commercially available 1-methyl-1H-pyrazol-3-amine (CAS: 1904-31-0, 39 μL, 0.50 mmol), HATU (233 mg, 0.61 mmol) and DIPEA (0.12 mL, 0.67 mmol) were stirred in DMF (1.7 mL) overnight. After workup, the crude product was purified using preparative HPLC (method 4) giving the desired amide 23 (54.0 mg, 44% yield). ¹H-NMR (400 MHz, DMSO-d₆) δ [ppm]: 2.518 (1.62), 2.523 (1.15), 3.785 (16.00), 6.547 (3.11), 6.553 (3.17), 6.979 (0.47), 6.982 (0.82), 6.985 (0.49), 7.001 (1.75), 7.016 (0.58), 7.019 (0.98), 7.022 (0.56), 7.335 (2.08), 7.339 (0.69), 7.353 (2.40), 7.357 (2.69), 7.370 (0.64), 7.375 (2.10), 7.626 (2.60), 7.632 (2.59), 7.674 (6.96), 7.677 (2.94), 7.679 (3.06), 7.694 (0.75), 7.698 (2.65), 7.702 (2.05), 9.800 (2.33), 10.389 (2.48); LC-MS (method 2): Rₜ=0.98 min; MS (ESIpos): m/z=300 [M+H]⁺.

### Example 24

### N-(5-tert-Butyl-1,2-oxazol-3-yl)-2-[(4-cyanophenyl)amino]-1,3-thiazole-4-carboxamide

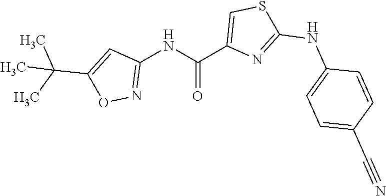 [SMILES: CC(C)(C)c1cc(NC(=O)c2csc(Nc3ccc(C#N)cc3)n2)no1]

According to GP1, commercially available 5-tert-butyl-1,2-oxazol-3-amine (CAS: 55809-36-4, 100 mg, 0.71 mmol), commercially available 2-[(4-cyanophenyl)amino]-1,3-thiazole-4-carboxylic acid (CAS: 1518535-90-4, 159 mg, 0.65 mmol), HATU (271 mg, 0.71 mmol) and DIPEA (0.12 mL, 0.71 mmol) were stirred in DMF (1.6 mL) overnight at RT and further 2 h at 40° C. After workup, the crud e product was purified using preparative HPLC (method 3) giving the desired amide 24 (137 mg, 50% yield) as a light orange solid. ¹H-NMR (400 MHz, DMSO-d₆) δ [ppm]: 1.327 (16.00), 2.518 (0.41), 6.701 (3.07), 7.756 (1.23), 7.778 (1.56), 7.930 (3.19), 7.934 (1.50), 7.956 (1.05); LC-MS (method 2): Rₜ=1.27 min; MS (ESIpos): m/z=368 [M+H]⁺.

### Example 25

### N-(5-tert-Butyl-1,2-oxazol-3-yl)-2-[(2-fluorophenyl)amino]-1,3-thiazole-4-carboxamide

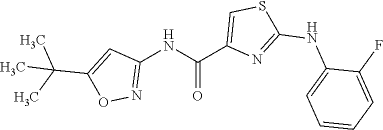 [SMILES: CC(C)(C)c1cc(NC(=O)c2csc(Nc3ccccc3F)n2)no1]

According to GP1, commercially available 5-tert-butyl-1,2-oxazol-3-amine (CAS: 55809-36-4, 100 mg, 0.71 mmol), commercially available 2-[(2-fluorophenyl)amino]-1,3-thiazole-4-carboxylic acid (CAS: 1176721-71-3, WO2012/37351 A1, 154 mg, 0.65 mmol), HATU (271 mg, 0.71 mmol) and DIPEA (0.12 mL, 0.71 mmol) were stirred in

DMF (1.6 mL) at RT overnight and further 2 h at 40° C. After workup, the crude product was purified using preparative HPLC (method 3) giving the desired amide 25 (132 mg (95%, 49% yield) as a colourless solid. ¹H-NMR (400 MHz, DMSO-d₆) δ [ppm]: 1.321 (16.00), 6.691 (2.29), 7.217 (0.41), 7.830 (1.95); LC-MS (method 2): Rₜ=1.36 min; MS (ESIneg): m/z=359 [M−H]⁻.

### Example 26

### 2-[(4-Chlorophenyl)amino]-N-[3-(3,4-dihydroisoquinolin-2(1H)-yl)propyl]-1,3-thiazole-4-carboxamide

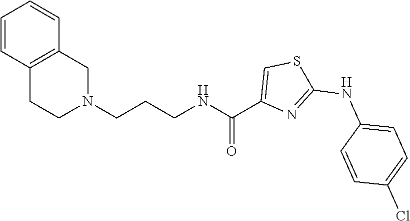 [SMILES: O=C(NCCCN1CCc2ccccc2C1)c1csc(Nc2ccc(Cl)cc2)n1]

According to GP1, commercially available 3-[3,4-dihydroisoquinolin-2(1H)-yl]propan-1-amine dihydrochloride (CAS: 93138-50-2, U.S. Pat. No. 6,235,731 B1, 2001, 114 mg, 0.43 mmol), 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 100 mg, 0.39 mmol), HATU (164 mg, 0.43 mmol) and DIPEA (0.10 mL, 0.59 mmol) were stirred in DMF (0.7 mL) for 3 d. After workup, the crude product was purified using preparative HPLC (method 3) giving the desired amide 26 (8.00 mg, 85% purity, 4% yield) as a colourless solid. ¹H-NMR (400 MHz, DMSO-d₆) δ [ppm]: 1.755 (0.51), 1.773 (1.72), 1.791 (2.55), 1.808 (1.83), 1.826 (0.58), 2.323 (0.58), 2.327 (0.81), 2.331 (0.59), 2.481 (3.50), 2.518 (4.42), 2.523 (2.48), 2.634 (1.99), 2.649 (5.16), 2.663 (3.29), 2.673 (0.78), 2.779 (2.32), 2.794 (3.73), 2.808 (1.60), 3.315 (1.29), 3.350 (2.71), 3.366 (1.13), 3.552 (8.12), 7.016 (1.16), 7.026 (1.67), 7.029 (1.78), 7.038 (2.60), 7.045 (1.70), 7.048 (1.94), 7.059 (2.91), 7.065 (2.28), 7.074 (6.88), 7.082 (3.54), 7.088 (3.26), 7.096 (2.88), 7.291 (0.90), 7.299 (8.30), 7.305 (2.49), 7.317 (2.49), 7.322 (9.68), 7.330 (0.86), 7.420 (0.75), 7.497 (16.00), 7.529 (0.74), 7.709 (0.92), 7.716 (9.14), 7.722 (2.49), 7.733 (2.37), 7.739 (7.74), 7.747 (0.66), 7.863 (0.50), 7.885 (0.43), 8.124 (1.04), 8.139 (2.18), 8.154 (1.00), 10.425 (1.28); LC-MS (method 2): Rₜ=1.36 min; MS (ESIneg): m/z=426 [M−H]⁻.

### Example 27

### {2-[(4-Chlorophenyl)amino]-1,3-thiazol-4-yl}{4-[3,4-dihydroisoquinolin-2(1H)-yl]piperidin-1-yl}methanone

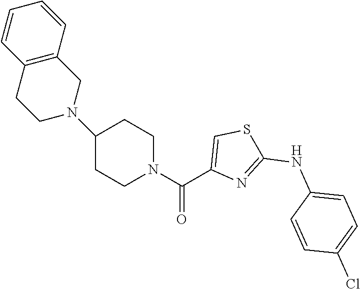 [SMILES: O=C(c1csc(Nc2ccc(Cl)cc2)n1)N1CCC(N2CCc3ccccc3C2)CC1]

According to GP1, commercially available 2-(piperidin-4-yl)-1,2,3,4-tetrahydroisoquinoline dihydrochloride (CAS: 871113-10-9, 125 mg, 0.43 mmol), commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 100 mg, 0.39 mmol), HATU (164 mg, 0.43 mmol) and DIPEA (0.10 mL, 0.59 mmol) were stirred in DMF (0.7 mL) for 3 d. After workup, the crude product was purified using preparative HPLC (method 3) giving the desired amide 27 (31.0 mg, 14% yield) as a colourless solid. ¹H-NMR (400 MHz, DMSO-d₆) δ [ppm]: 1.531 (0.83), 1.878 (1.27), 2.332 (0.48), 2.518 (2.46), 2.523 (1.73), 2.540 (0.80), 2.674 (0.52), 2.706 (0.64), 2.715 (0.52), 2.734 (1.20), 2.742 (0.88), 2.782 (11.30), 3.718 (6.18), 4.370 (0.46), 4.462 (0.48), 7.039 (0.80), 7.047 (1.78), 7.056 (1.99), 7.060 (2.10), 7.077 (3.35), 7.083 (10.14), 7.089 (3.21), 7.092 (3.59), 7.106 (1.37), 7.327 (16.00), 7.334 (0.77), 7.342 (8.06), 7.347 (2.12), 7.359 (2.44), 7.364 (8.87), 7.372 (0.86), 7.635 (0.89), 7.643 (8.81), 7.648 (2.34), 7.660 (2.21), 7.666 (7.62), 7.674 (0.66), 10.488 (2.26); LC-MS (method 2): Rₜ=1.36 min; MS (ESIneg): m/z=452 [M−H]⁻.

### Example 28

### N-[1-(2-Hydroxyethyl)-1H-pyrazol-3-yl]-2-(phenylamino)-1,3-thiazole-4-carboxamide

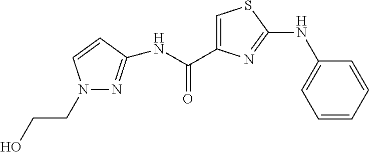 [SMILES: O=C(Nc1ccn(CCO)n1)c1csc(Nc2ccccc2)n1]

According to GP1, commercially available 2-(phenylamino)-1,3-thiazole-4-carboxylic acid (CAS: 165683-01-2, WO2011/109059A1, 90.0 mg, 0.41 mmol), 2-(3-amino-1H-pyrazol-1-yl)ethanol (CAS: 84407-13-6, 62.3 mg, 0.490 mmol), HATU (233 mg, 0.61 mmol) and DIPEA (0.12 mL, 0.67 mmol) were stirred in DMF (1.7 mL) overnight. After workup, the crude product was purified using preparative HPLC (method 4) giving the desired amide 28 (53.0 mg, 90% purity, 35% yield). ¹H-NMR (400 MHz, DMSO-d₆) δ [ppm]: 2.518 (2.28), 2.523 (1.54), 3.707 (1.51), 3.721 (4.58), 3.734 (4.77), 3.748 (1.78), 4.059 (3.32), 4.073 (6.26), 4.088 (2.85), 4.885 (2.43), 4.898 (5.71), 4.911 (2.32), 6.558 (5.64), 6.563 (5.50), 6.979 (0.86), 6.982 (1.56), 6.984 (0.92), 7.000 (3.24), 7.016 (1.07), 7.018 (1.81), 7.021 (1.02), 7.330 (0.49), 7.336 (3.74), 7.340 (1.26), 7.354 (4.37), 7.357 (4.83), 7.370 (1.17), 7.376 (3.72), 7.642 (5.08), 7.647 (5.01), 7.679 (16.00), 7.700 (4.89), 7.703 (3.83), 9.834 (4.84), 10.386 (4.63); LC-MS (method 2): Rₜ=0.88 min; MS (ESIneg): m/z=328 [M−H]⁻.

### Example 29

### N-(5-tert-Butyl-4-methyl-1,2-oxazol-3-yl)-2-[(4-chlorophenyl)amino]-1,3-thiazole-4-carboxamide

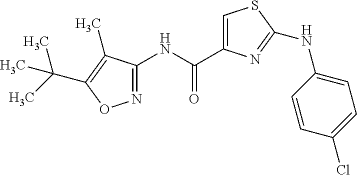 [SMILES: Cc1c(NC(=O)c2csc(Nc3ccc(Cl)cc3)n2)noc1C(C)(C)C]

According to GP1, commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 70.0 mg, 0.28 mmol), commercially available 5-tert-butyl-4-methyl-1,2-oxazol-3-amine (CAS: 120771-20-2, 39.3 mg, 98% purity, 0.25 mmol), HATU (105 mg, 0.28 mmol) and DIPEA (0.05 mL, 0.27 mmol) were stirred in DMF (2.0 mL) for 7 d. After workup, the crude product was purified using preparative HPLC (method 3) giving the desired amide 29 (4.00 mg, 85% purity, 3% yield). ¹H-NMR (400 MHz, DMSO-d₆) δ [ppm]: 1.233 (0.62), 1.287 (0.41), 1.335 (16.00), 1.959 (5.64), 2.332 (0.59), 2.518 (3.27), 2.522 (2.17), 2.673 (0.61), 7.311 (1.61), 7.316 (0.45), 7.328 (0.49), 7.333 (1.71), 7.780 (2.71), 7.823 (1.24), 7.846 (1.17); LC-MS (method 2): Rₜ=0.88 min; MS (ESIneg): m/z=328 [M−H]⁻.

### Example 30

### N-(4-Cyano-1H-pyrazol-3-yl)-2-(phenylamino)-1,3-thiazole-4-carboxamide

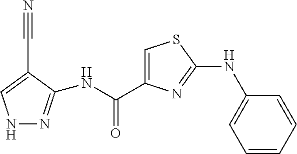 [SMILES: N#Cc1c[nH]nc1NC(=O)c1csc(Nc2ccccc2)n1]

According to GP1, commercially available 2-(phenylamino)-1,3-thiazole-4-carboxylic acid (CAS: 165683-01-2, WO2011/109059A1, 90.0 mg, 0.41 mmol), 3-amino-1H-pyrazole-4-carbonitrile (CAS: 1204396-41-7, 16617-46-2, 53.0 mg, 0.49 mmol), HATU (233 mg, 0.61 mmol) and DIPEA (0.12 mL, 0.67 mmol) were stirred in DMF (1.7 mL) overnight. After workup, the crude product was purified using preparative HPLC (method 4) giving the desired amide 30 (53.0 mg, 42% yield). ¹H-NMR (400 MHz, DMSO-d₆) δ [ppm]: 2.074 (1.03), 2.337 (0.53), 2.518 (6.32), 2.523 (4.45), 2.674 (1.16), 2.678 (0.53), 6.965 (3.16), 6.968 (1.89), 6.984 (6.71), 7.002 (3.79), 7.271 (0.55), 7.298 (1.00), 7.303 (7.97), 7.308 (2.79), 7.322 (9.58), 7.325 (10.55), 7.339 (2.53), 7.343 (7.79), 7.761 (16.00), 7.767 (9.61), 7.769 (10.24), 7.788 (9.16), 8.518 (0.82), 10.229 (0.42), 10.401 (5.87); LC-MS (method 2): Rₜ=0.91 min; MS (ESIneg): m/z=309 [M−H]⁻.

### Example 31

### 2-[(4-Chlorophenyl)amino]-N-[1-(oxetan-3-yl)-1H-pyrazol-4-yl]-1,3-thiazole-4-carboxamide

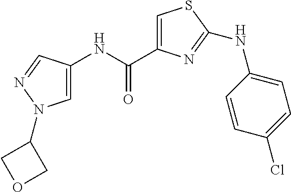 [SMILES: O=C(Nc1cnn(C2COC2)c1)c1csc(Nc2ccc(Cl)cc2)n1]

According to GP1, commercially available 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 70.0 mg, 97% purity, 0.27 mmol), commercially available 1-(oxetan-3-yl)-1H-pyrazol-4-amine (CAS: 1338719-26-8, 40.8 mg, 0.29 mmol), HATU (112 mg, 0.29 mmol) and DIPEA (51 μL, 0.29 mmol) were stirred in DMF (2.0 mL) over the weekend. After workup, the crude product was purified using preparative HPLC (method 3) giving the desired amide 31 (49.4 mg, 47% yield) as a colourless solid. ¹H-NMR (400 MHz, DMSO-d₆) δ [ppm]: 2.075 (1.91), 2.518 (4.08), 2.522 (2.72), 4.860 (2.70), 4.877 (9.47), 4.894 (8.60), 4.915 (7.89), 4.932 (2.96), 5.558 (0.67), 5.577 (1.62), 5.592 (2.76), 5.611 (1.50), 5.627 (0.53), 7.302 (0.75), 7.347 (0.79), 7.355 (9.02), 7.361 (2.51), 7.372 (2.72), 7.378 (10.20), 7.386 (0.81), 7.629 (0.55), 7.647 (16.00), 7.784 (0.93), 7.791 (9.79), 7.797 (2.78), 7.808 (2.56), 7.814 (8.74), 7.822 (0.77), 7.845 (9.31), 8.204 (9.13), 10.070 (5.72), 10.495 (4.99); LC-MS (method 2): Rₜ=1.03 min; MS (ESIpos): m/z=375 [M+H]⁺.

### Example32

### N-(5-tert-Butyl-1,2-oxazol-3-yl)-2-(cyclopropylamino)-1,3-thiazole-4-carboxamide

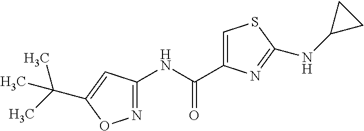 [SMILES: CC(C)(C)c1cc(NC(=O)c2csc(NC3CC3)n2)no1]

INT-6 (50.0 mg, 0.15 mmol) and commercially available cyclopropanamine (CAS: 765-30-0, 43.2 mg, 0.76 mmol) were suspended in pyridine (1 mL) under argon atmosphere. The mixture was heated to 120° C. and stirred overnight. The crude product was then dissolved in dichloromethane, washed with aqueous sodium carbonate solution, water and brine and evaporated under vacuum. The obtained residue was purified using preparative HPLC (method 3) giving the desired product 32 (3.60 mg, 7% yield) as a colourless solid. ¹H-NMR (400 MHz, DMSO-d₆) δ [ppm]: 0.532 (0.50), 0.537 (0.52), 0.541 (0.58), 0.547 (0.52), 0.747 (0.48), 0.753 (0.63), 0.764 (0.59), 0.769 (0.46), 1.308 (16.00), 1.313 (1.25), 2.518 (0.56), 6.670 (2.29), 7.612 (1.27), 7.614 (1.26), 8.262 (0.43), 10.077 (0.59); LC-MS (method 2): Rₜ=1.24 min; MS (ESIpos): m/z=306 [M+H]⁺.

### Example 33

### {2-[(4-Chlorophenyl)amino]-1,3-thiazol-4-yl}[4-(5-methyl-1,3-benzothiazol-2-yl)piperazin-1-yl]methanone

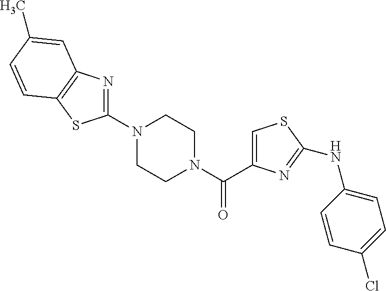 [SMILES: Cc1ccc2sc(N3CCN(C(=O)c4csc(Nc5ccc(Cl)cc5)n4)CC3)nc2c1]

According to GP1, commercially available 5-methyl-2-(piperazin-1-yl)-1,3-benzothiazole (CAS: 55745-83-0, Bio. Med. Chem., 2015, 23, 7661-7670, 50.0 mg, 0.21 mmol), 2-[(4-chlorophenyl)amino]-1,3-thiazole-4-carboxylic acid (CAS: 165682-82-6, WO2007/5785 A1, Bio. Med. Chem. Let., 2013, 23, 4979-4984, 54.6 mg, 0.21 mmol), HATU (89.6 mg, 0.24 mmol) and DIPEA (41 μL, 0.24 mmol) were stirred in DMF (1 mL). After workup, the crude product was purified using preparative HPLC (method 3) giving the desired amide 33 (5.0 mg, 4% yield) as a colourless solid. ¹H-NMR (400 MHz, DMSO-d₆) δ [ppm]: 2.310 (0.81), 2.323 (1.28), 2.327 (1.02), 2.332 (0.72), 2.348 (16.00), 2.518 (2.31), 2.523 (1.55), 2.540 (0.85), 2.669 (0.56), 2.902 (1.50), 2.955 (1.57), 3.648 (2.99), 3.659 (4.71), 3.673 (4.13), 3.732 (0.87), 3.784 (0.80), 3.936 (0.73), 6.908 (2.02), 6.911 (2.02), 6.929 (2.16), 6.931 (2.14), 7.308 (3.61), 7.310 (3.87), 7.362 (0.61), 7.371 (7.74), 7.376 (2.43), 7.388 (2.37), 7.393 (8.56), 7.401 (0.97), 7.433 (15.01), 7.634 (0.99), 7.641 (11.42), 7.647 (2.59), 7.660 (5.66), 7.664 (7.45), 7.672 (0.80), 7.827 (0.45), 7.849 (0.41), 10.531 (1.71); LC-MS (method 2): Rₜ=1.43 min; MS (ESIneg): m/z=469 [M−H]⁻.

The example compounds 34-53 were prepared according to the following procedure in analogy to GP2: To a solution of 1 mmol amine in THF (0.625 M, 400 μL, 4 eq) was added 2 mmol sodium hydride (8 eq) and the mixture was stirred for 30 min at RT. After addition of 0.25 mmol bromide INT-2 in THF (0.4 M, 400 μL), the mixture was heated at 70° C. overnight, evaporated in vaccuo, redissolved in DMSO and filtered. The filter was washed with ACN/water (9:1) and the combined filtrates were evaporated in vaccuo. The residue was dissolved in DMSO and subjected to preparative HPLC to yield the target compounds. The following commercially available amines were used: 2-amino-5-picoline (CAS: 1603-41-4), 2-amino-chloropyridin (CAS: 1072-98-6), 2-chloropyridin-2-amine (CAS: 19798-80-2), 2-phenylethanamine (CAS: 64-04-0), 2,3-dihydro-1H-inden-5-amine (CAS: 24425-40-9), 4-chloro-3-fluoroaniline (CAS: 367-22-6), benzofurane-6-amine (CAS: 110677-54-8), 2-fluoro-4-methylaniline (CAS: 452-80-2), 1-(3-fluorophenyl)methanamine (CAS: 100-82-3), m-toluidin (CAS: 108-44-1), 2-aminomethylfurane (CAS: 617-89-0); 3,4-difluorobenzamine (CAS: 3863-11-4), 3-chloro-4-fluoro-phenylamine (CAS: 367-21-5), 2-amino-N-isoquinolin-3-yl-benzamide (CAS: 267891-89-4), m-chloroaniline (CAS: 108-42-9), 2-aminomethyltetrahydrofurane (CAS: 4795-29-3), 4-fluoro-2-methylaniline (CAS: 452-71-1), 3-ethynylaniline (CAS: 54060-30-9), 3-ethynylaniline (CAS: 54060-30-9), 2,3-difluoroaniline (CAS: 4519-40-8), 2-fluoro-5-aminopyridine (CAS: 1827-27-6).

| Ex- | |
|---|---|
| ample | Structure, name and analytics |
| 34 | 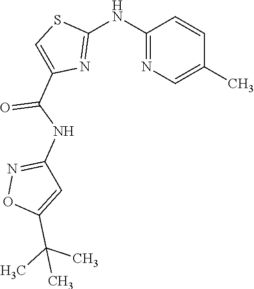 [SMILES: Cc1ccc(Nc2nc(C(=O)Nc3cc(C(C)(C)C)on3)cs2)nc1] |
| | N-(5-tert-Butyl-1,2-oxazol-3-yl)-2-[(5-methylpyridin-2-yl) |
| | amino]-1,3-thiazole-4-carboxamide |
| | LC-MS (method 5): Rₜ = 1.32 min; MS (ESIpos): m/z = 358 |
| | [M + H⁺] |
| 35 | 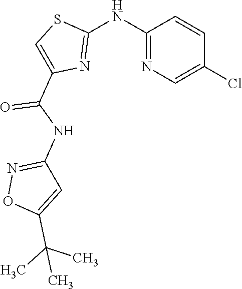 [SMILES: CC(C)(C)c1cc(NC(=O)c2csc(Nc3ccc(Cl)cn3)n2)no1] |
| | N-(5-tert-Butyl-1,2-oxazol-3-yl)-2-[(5-chloropyridin-2-yl)amino]- |
| | 1,3-thiazole-4-carboxamide |
| | LC-MS (method 5): Rₜ = 1.40 min; MS (ESIpos): m/z = 379 |
| | [M + H⁺] |
| 36 | 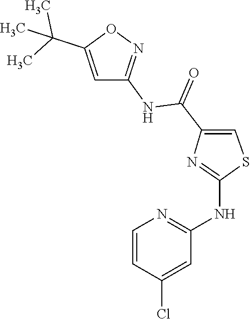 [SMILES: CC(C)(C)c1cc(NC(=O)c2csc(Nc3cc(Cl)ccn3)n2)no1] |
| | N-(5-tert-Butyl-1,2-oxazol-3-yl)-2-[(4-chloropyridin-2-yl)amino]- |
| | 1,3-thiazole-4-carboxamide |
| | LC-MS (method 5): Rₜ = 1.38 min; MS (ESIpos): m/z = 379 |
| | [M + H⁺] |
| 37 | 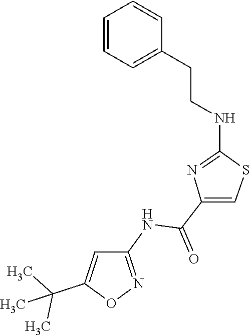 [SMILES: CC(C)(C)c1cc(NC(=O)c2csc(NCCc3ccccc3)n2)no1] |
| | N-(5-tert-Butyl-1,2-oxazol-3-yl)-2-[(2-phenylethyl)amino]-1,3- |
| | thiazole-4-carboxamide |
| | LC-MS (method 5): Rₜ = 1.41 min; MS (ESIpos): m/z = 372 |
| | [M + H⁺] |
| 38 | 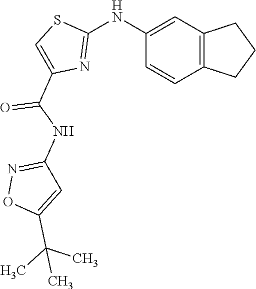 [SMILES: CC(C)(C)c1cc(NC(=O)c2csc(Nc3ccc4c(c3)CCC4)n2)no1] |
| | N-(5-tert-Butyl-1,2-oxazol-3-yl)-2-(2,3-dihydro-1H-inden-5- |
| | ylamino)-1,3-thiazole-4-carboxamide |
| | LC-MS (method 5): Rₜ = 1.49 min; MS (ESIpos): m/z = 383 |
| | [M + H⁺] |
| 39 | 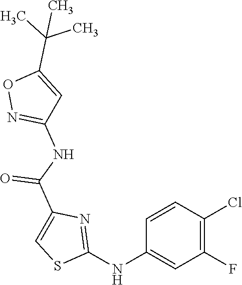 [SMILES: CC(C)(C)c1cc(NC(=O)c2csc(Nc3ccc(Cl)c(F)c3)n2)no1] |
| | N-(5-tert-Butyl-1,2-oxazol-3-yl)-2-[(4-chloro-3-fluorophenyl) |
| | amino]-1,3-thiazole-4-carboxamide |
| | LC-MS (method 5): Rₜ = 1.32 min; MS (ESIpos): m/z = 396 |
| | [M + H⁺] |
| 40 | 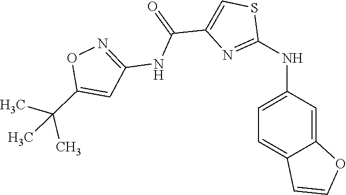 [SMILES: CC(C)(C)c1cc(NC(=O)c2csc(Nc3ccc4ccoc4c3)n2)no1] |
| | 2-(1-Benzofuran-6-ylamino)-N-(5-tert-butyl-1,2-oxazol-3-yl)- |
| | 1,3-thiazole-4-carboxamide |
| | LC-MS (method 5): Rₜ = 1.36 min; MS (ESIpos): m/z = 383 |
| | [M + H⁺] |
| 41 | 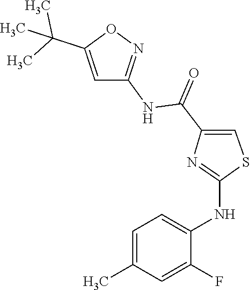 [SMILES: Cc1ccc(Nc2nc(C(=O)Nc3cc(C(C)(C)C)on3)cs2)c(F)c1] |
| | N-(5-tert-Butyl-1,2-oxazol-3-yl)-2-[(2-fluoro-4-methylphenyl) |
| | amino]-1,3-thiazole-4-carboxamide |
| | LC-MS (method 5): Rₜ = 1.41 min; MS (ESIpos): m/z = 375 |
| | [M + H⁺] |
| 42 | 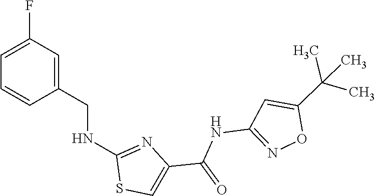 [SMILES: CC(C)(C)c1cc(NC(=O)c2csc(NCc3cccc(F)c3)n2)no1] |
| | N-(5-tert-Butyl-1,2-oxazol-3-yl)-2-[(3-fluorobenzyl)amino]-1,3- |
| | thiazole-4-carboxamide |
| | LC-MS (method 5): Rₜ = 1.35 min; MS (ESIpos): m/z = 376 |
| | [M + H⁺] |
| 43 | 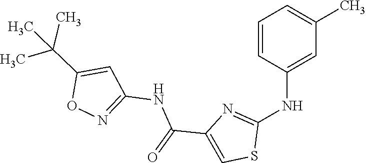 [SMILES: Cc1cccc(Nc2nc(C(=O)Nc3cc(C(C)(C)C)on3)cs2)c1] |
| | N-(5-tert-Butyl-1,2-oxazol-3-yl)-2-[(3-methylphenyl)amino]-1,3- |
| | thiazole-4-carboxamide |
| | LC-MS (method 5): Rₜ = 1.40 min; MS (ESIpos): m/z = 357 |
| | [M + H⁺] |
| 44 | 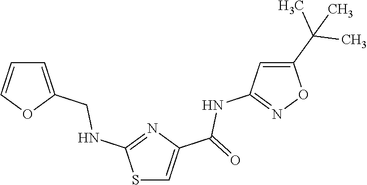 [SMILES: CC(C)(C)c1cc(NC(=O)c2csc(NCc3ccco3)n2)no1] |
| | N-(5-tert-Butyl-1,2-oxazol-3-yl)-2-[(furan-2-ylmethyl)amino]- |
| | 1,3-thiazole-4-carboxamide |
| | LC-MS (method 5): Rₜ = 1.27 min; MS (ESIpos): m/z = 347 |
| | [M + H⁺] |
| 45 | 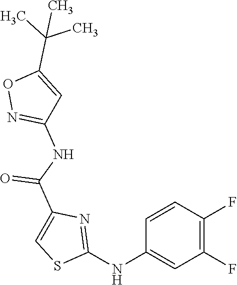 [SMILES: CC(C)(C)c1cc(NC(=O)c2csc(Nc3ccc(F)c(F)c3)n2)no1] |
| | N-(5-tert-Butyl-1,2-oxazol-3-yl)-2-[(3,4-difluorophenyl)amino]- |
| | 1,3-thiazole-4-carboxamide |
| | LC-MS (method 5): Rₜ = 1.37 min; MS (ESIpos): m/z = 379 |
| | [M + H⁺] |
| 46 | 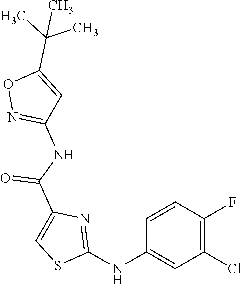 [SMILES: CC(C)(C)c1cc(NC(=O)c2csc(Nc3ccc(F)c(Cl)c3)n2)no1] |
| | N-(5-tert-Butyl-1,2-oxazol-3-yl)-2-[(3-chloro-4-fluorophenyl) |
| | amino]-1,3-thiazole-4-carboxamide |
| | LC-MS (method 5): Rₜ = 1.43 min; MS (ESIpos): m/z = 396 |
| | [M + H⁺] |
| 47 | 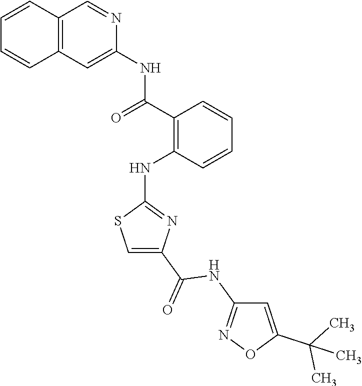 [SMILES: CC(C)(C)c1cc(NC(=O)c2csc(Nc3ccccc3C(=O)Nc3cc4ccccc4cn3)n2)no1] |
| | N-(5-tert-Butyl-1,2-oxazol-3-yl)-2-{[2-(isoquinolin-3- |
| | ylcarbamoyl)phenyl]amino}-1,3-thiazole-4-carboxamide |
| | LC-MS (method 5): Rₜ = 1.49 min; MS (ESIpos): m/z = 514 |
| | [M + H⁺] |
| 48 | 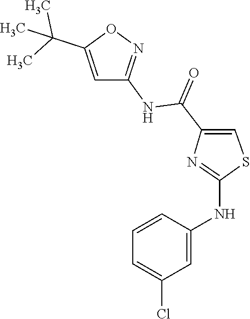 [SMILES: CC(C)(C)c1cc(NC(=O)c2csc(Nc3cccc(Cl)c3)n2)no1] |
| | N-(5-tert-Butyl-1,2-oxazol-3-yl)-2-[(3-chlorophenyl)amino]-1,3- |
| | thiazole-4-carboxamide |
| | LC-MS (method 5): Rₜ = 1.42 min; MS (ESIpos): m/z = 378 |
| | [M + H⁺] |
| 49 | 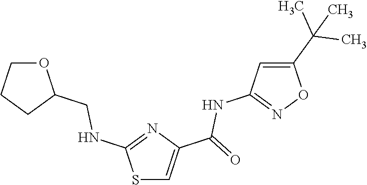 [SMILES: CC(C)(C)c1cc(NC(=O)c2csc(NCC3CCCO3)n2)no1] |
| | (rac)-N-(5-tert-Butyl-1,2-oxazol-3-yl)-2-{[-tetrahydrofuran-2- |
| | ylmethyl]amino}-1,3-thiazole-4-carboxamide |
| | LC-MS (method 5): Rₜ = 1.21 min; MS (ESIpos): m/z = 351 |
| | [M + H⁺] |
| 50 | 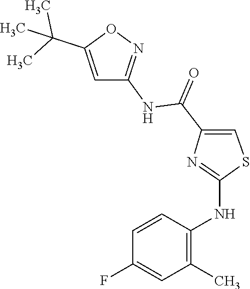 [SMILES: Cc1cc(F)ccc1Nc1nc(C(=O)Nc2cc(C(C)(C)C)on2)cs1] |
| | N-(5-tert-Butyl-1,2-oxazol-3-yl)-2-[(4-fluoro-2-methylphenyl) |
| | amino]-1,3-thiazole-4-carboxamide |
| | LC-MS (method 5): Rₜ = 1.38 min; MS (ESIpos): m/z = 375 |
| | [M + H⁺] |
| 51 | 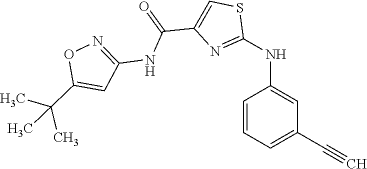 [SMILES: C#Cc1cccc(Nc2nc(C(=O)Nc3cc(C(C)(C)C)on3)cs2)c1] |
| | N-(5-tert-Butyl-1,2-oxazol-3-yl)-2-[(3-ethynylphenyl)amino]-1,3- |
| | thiazole-4-carboxamide |
| | LC-MS (method 5): Rₜ = 1.36 min; MS (ESIpos): m/z = 367 |
| | [M + H⁺] |
| 52 | 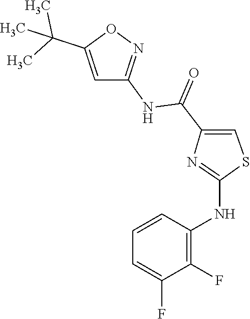 [SMILES: CC(C)(C)c1cc(NC(=O)c2csc(Nc3cccc(F)c3F)n2)no1] |
| | N-(5-tert-Butyl-1,2-oxazol-3-yl)-2-[(2,3-difluorophenyl)amino]- |
| | 1,3-thiazole-4-carboxamide |
| | LC-MS (method 5): Rₜ = 1.37 min; MS (ESIpos): m/z = 379 |
| | [M + H⁺] |
| 53 | 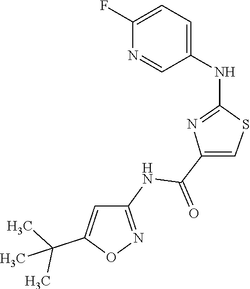 [SMILES: CC(C)(C)c1cc(NC(=O)c2csc(Nc3ccc(F)nc3)n2)no1] |
| | N-(5-tert-Butyl-1,2-oxazol-3-yl)-2-[(6-fluoropyridin-3-yl)amino]- |
| | 1,3-thiazole-4-carboxamide |
| | LC-MS (method 5): Rₜ = 1.22 min; MS (ESIpos): m/z = 362 |
| | [M + H⁺] |

### EXPERIMENTAL SECTION

### Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

The in vitro activity of the compounds of the present invention can be demonstrated in the following assays:

In Vitro Assay 1: PRMT5 SPA Assay (“EAA”)

PRMT5 inhibitory activities of the compounds described in the present invention were quantified using a scintillation proximity assay (SPA) which measures methylation by the enzyme of a synthetic, biotinylated peptide corresponding to the N-terminal sequence of Histone H4 (a.a. 1-24). The peptide of sequence SGRGKGGKGLGKGGAKRHRKVLRD-K(Btn)-amide is commercially available (e.g. from Biosyntan) and will be herein referred to as H4(1-24). The PRMT5:MEP50 protein complex used in the assay was produced in house following standard protocols. Briefly, full length PRMT5 and MEP50 constructs respectively tagged with N-terminal FLAG and 6× His sequences were co-expressed in Hi5 insect cells, and the recombinant protein complex was purified by FLAG affinity and size exclusion chromatography.

In a typical assay 11 different concentrations of each compound (0.1 nM, 0.33 nM, 1.1 nM, 3.8 nM, 13 nM, 44 nM, 0.15 μM, 0.51 μM, 1.7 μM, 5.9 μM and 20 μM) were tested in duplicate within the same microtiter plate. To this end, 100-fold concentrated compound solutions (in DMSO) were previously prepared by serial dilution (1:3.4) of 2 mM stocks in a clear low volume 384-well source microtiter plate (Greiner Bio-One), from which 50 nl of compounds were transferred into a white low volume test microtiter plate from the same supplier. Subsequently, 2.5 μL PRMT5 in aqueous assay buffer [20 mM Bicine pH 8.0 (Alfa Aesar), 7.5 mM Tris(2-carboxyethyl) phosphine hydrochloride (TCEP, Sigma), 0.1% (w/v) bovine gamma globuline (BGG, Sigma), 0.002% (v/v) Tween-20 (Sigma)] were added to the compounds in the test plate to a final enzyme concentration of—typically—7.5 nM (this parameter was adjusted depending on the activity of the enzyme lot in order to be within the linear dynamic range of the assay). The samples were then incubated for 15 min at 22° C. to allow pre-equilibration of the putative enzyme-inhibitor complexes before the start of the methylation reaction, which was initiated by the addition of 2.5 μL 2-fold concentrated solution (in assay buffer) of “cold” S-Adenosyl-L-Methionine (SAM, Sigma, final concentration: 0.5 μM), tritiated 3H-SAM (Perkin Elmer, final concentration: 0.5 μM) and H4(1-24) peptide substrate (final concentration: 0.06 μM). The resulting mixture (5 μL final volume) was shortly centrifuged (2 min., 1500 rpm) and incubated at 22° C. during 60 min. Thereupon the reaction was stopped by adding 3 μL of Streptavidin PS SPA imaging beads (Perkin Elmer, final concentration of 0.375 μg/μL) and “cold” SAM (AK Scientific, 250 μM final concentration) for non-specific binding reduction. Plates containing the stopped reaction were sealed with transparent adhesive foil (Perkin Elmer), centrifuged (2 min., 1500 rpm), and further incubated either 1 h at 22° C. or overnight at 4° C. in order to allow the SPA signals to develop. Subsequently, the amount of product was evaluated by measuring the energy transfer from the β-particles emitted by the 3H-labeled substrate to the Europium scintillator co-polymerized in the polystyrene matrix of the PS imaging beads, using the standard settings for this purpose of a Viewlux (Perkin-Elmer) CCD plate imaging device (emission filter 613/55 (IFP).

The resulting scintillation counts were taken as indicator for the amount of methylated peptide per well. The data were normalised using two sets of control wells (typically 16 each) for high—(=enzyme reaction with DMSO instead of test compound=0%=minimum inhibition) and low—(=all assay components without enzyme=100%=maximum inhibition) PRMT5 activity. IC₅₀ values were calculated by fitting the normalized inhibition data to a 4-parameter logistic equation using the “Screener” analysis software from Genedata.

In Vitro Assay 2: Quantification of SDMA (Symmetric Dimethylarginine) Expression Levels (H4 Arg3) After Small Molecule Treatment of Cancer Cells

The SDMA-assay based on High-Content Analysis (HCA) enables the biological characterization of compounds inhibiting PRMT5. By this assay, substances are benchmarked on their cellular activity to modulate histone H4 Arg3 levels over broad inhibitor concentration ranges.

Cultivated cells of the human mammary gland tumor cell line MDA-MB-468 (ATCC HTB-132) were plated at a density of 2500 cells per well in a 384-well microtiter plate in 20 μL of Roswell Park Memorial Institute (RPMI) 1640 medium supplemented with 1% (v/v) glutamine, 1% (v/v) penicillin, 1% (v/v) streptomycin and 10% (v/v) fetal calf serum. After pre-incubation at 37° C. overnight, test compo unds solubilized in DMSO were added at various concentrations (0 μM, as well as in the range of 0.005 μM-30 μM; the final concentration of the solvent DMSO was 0.5% (v/v)). Cells were incubated at 37° C. for 72 h in the presence of test compounds. Thereafter, cells were fixed with 4% (v/v) paraformaldehyde in phosphate buffered saline (PBS) at room temperature for 1 h, permeabilized using 0.5% (v/v) Triton X™ 100 in PBS for 15 min and blocked with 1% (v/v) bovine serum albumin (BSA) in PBS for 30 min. In the following, cells were incubated with anti-dimethyl-histone H4 (Arg3) symmetric antibody (Millipore #07-947) at RT for 2 h. After a single washing step with blocking solution, cells were incubated with species-specific fluorescent conjugated secondary antibody Alexa Fluor™ 488 (Jackson Immuno Research #111-546-003) at RT for 2 h.

Image acquisition for cells was carried out using the confocal imaging system OPERA® (PerkinElmer). Images were captured using two channels: Hoechst 33342 stain (DNA) and Alexa Fluor™ 488 (SDMA staining); and were analyzed using the MetaXpress® software (Molecular Devices). Exposure times were chosen for individual experiments to prevent saturation and allow for quantification in the linear range. A minimum of 500 cells in 5 field views were analyzed per well using 10× water immersion objective with a two-fold binning.

The image analysis routine was developed in MetaXpress®. The standardized image-analysis module “Count Nuclei” and the images of the DNA-channel were used for nuclei segmentation. The segmented nuclei were filtered for size, shape and signal intensity to exclude possible apoptotic and non-viable cells along with metaphase nuclei. These phenotypes result in overestimated antibody signals, due to their strong chromatin compaction. For further processing, binary object masks were calculated from the segmented and positively selected nuclei. The binary masks served as templates to be superimposed over the images of the SDMA channel. The SDMA average signal intensity per cell was quantified for every image set. The assay raw data were further analyzed by four parameter logistic regression analysis using Genedata's Assay Analyzer® and Condoseo® software to determine the IC₅₀ value for each tested compound.

In Vitro Assay 3: Determination of Cell Number After Small Molecule Treatment of Cancer Cells

To determine total cell number by nuclear count, chromatin is labeled with Hoechst 33342 stain at room temperature for 10 min of the samples as described in in vitro assay 2. Cells are washed with PBS and stored in PBS at 4° C. until analysis. Image acquisition for cells is carried out as described in in vitro assay 2.

TABLE 1
IC₅₀ values of examples in in vitro assay 1

| | PRMT5 |
|---|---|
| Example | IC₅₀ [M] |
| 1 | 5,48E-8 |
| 2 | 7,80E-8 |
| 3 | 1,77E-7 |
| 4 | 1,04E-7 |
| 5 | 1,13E-7 |
| 6 | 1,18E-7 |
| 7 | 1,49E-7 |
| 8 | 1,11E-7 |
| 9 | 2,39E-7 |
| 10 | 2,38E-7 |
| 11 | 2,80E-7 |
| 12 | 2,95E-7 |
| 13 | 3,44E-7 |
| 14 | 3,03E-7 |
| 15 | 4,00E-7 |
| 16 | 4,38E-7 |
| 17 | 5,85E-7 |
| 18 | 8,19E-7 |
| 19 | 7,75E-7 |
| 20 | 9,71E-7 |
| 21 | 1,39E-6 |
| 22 | 9,41E-7 |
| 23 | 1,61E-6 |
| 24 | 2,54E-6 |
| 25 | 2,49E-6 |
| 26 | 7,85E-6 |
| 27 | 7,61E-6 |
| 28 | 9,10E-6 |
| 29 | 9,68E-6 |
| 30 | 1,20E-5 |
| 31 | 1,51E-5 |
| 32 | 1,47E-5 |
| 33 | ! 2,97E-5 |
| 34 | 4,18E-8 |
| 35 | 4,96E-8 |
| 36 | 1,05E-7 |
| 37 | 1,19E-7 |
| 38 | 1,60E-7 |
| 39 | 1,83E-7 |
| 40 | 3,50E-7 |
| 41 | 4,26E-7 |
| 42 | 5,08E-7 |
| 43 | 5,12E-7 |
| 44 | 6,06E-7 |
| 45 | 8,15E-7 |
| 46 | 1,20E-6 |
| 47 | 1,30E-6 |
| 48 | 2,99E-6 |
| 49 | 5,68E-6 |
| 50 | 6,71E-6 |
| 51 | 1,05E-5 |
| 52 | 1,23E-5 |
| 53 | 1,52E-5 |

TABLE 2
IC₅₀ values of examples in in vitro assay 2

| | PRMT5 |
|---|---|
| Example | IC₅₀ [M] |
| 1 | 3,00E-5 |
| 2 | 2,45E-6 |
| 3 | 2,54E-5 |
| 4 | 2,08E-5 |
| 5 | 3,00E-5 |
| 7 | 1,76E-5 |
| 8 | 3,00E-5 |
| 10 | 2,79E-5 |
| 12 | 2,21E-5 |
| 13 | 2,61E-5 |
| 14 | 1,86E-5 |
| 15 | 3,00E-5 |
| 17 | 1,00E-5 |
| 18 | 3,00E-5 |
| 19 | 3,00E-5 |
| 20 | 3,00E-5 |
| 21 | 2,67E-5 |
| 22 | 2,34E-5 |
| 23 | 2,32E-5 |

1. A compound of general formula (I): 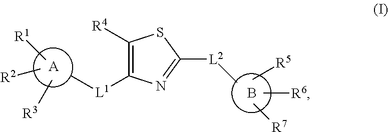
in which:
R¹ is selected from a hydrogen atom, a halogen atom, a C₁-C₆-alkyl group, a C₁-C₆-haloalkyl group, a C₃-C₆-cycloalkyl group, a C₁-C₆-alkoxy group and a phenyl group which is optionally substituted with a halogen atom, a C₁-C₄-alkoxy group, a C₁-C₄-alkyl group;
R² is selected from a hydrogen atom, a C₁-C₆-alkyl group, a —O—(CH₂)₂—NR⁸R⁹ group and a cyano group;
R³ is selected from a hydrogen atom, a cyano group, a NR⁸R⁹ group, a C₁-C₆-alkyl group, a C₁-C₆-hydroxyalkyl group and a heterocycloalkyl group;
R⁴ is hydrogen;
R⁵ is selected from a hydrogen atom, a halogen atom, a cyano group, a C₁-C₆-alkyl group and a N(O)₂ group;
R⁶ is selected from a hydrogen atom, a halogen atom, a C₁-C₆-alkyl group, a C₂-C₆-alkenyl group and a C₂-C₆-alkynyl group;
R⁷ is selected from a hydrogen atom, a halogen atom and a C₁-C₆-alkyl group;
R⁸, R⁹ is independently selected from a hydrogen atom and a C₁-C₆-alkyl group;
A is selected from a monocyclic or bicyclic aryl group, a partially saturated monocyclic or bicyclic aryl group, a monocyclic or bicyclic heteroaryl group, a partially saturated monocyclic or bicyclic heteroaryl group, 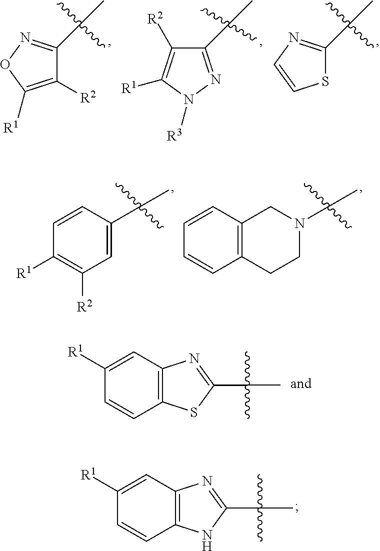
L¹ is selected from ^{$}—NH—C(O)—^{$$}, ^{$}—(CH₂)₃—NH—C(O)—^{$$}, ^{$}—(CH₂)₄—NH—C(O)—^{$$}, 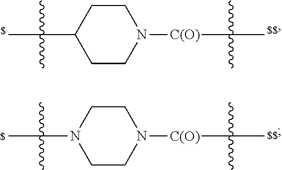
^{$} is the point of attachment of L¹ to ring A and ^{$$} is the point of attachment of L¹ to the thiazole carbon atom;
L² is selected from a bond, *—NH—**, *—NH—CH₂—**, *—NH—(CH₂)₂—**, 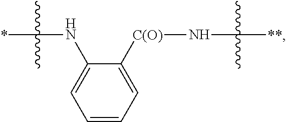
*—CH₂—**; and * is the point of attachment of L² to the thiazole carbon atom and ** is the point of attachment of L² to ring B;
B is selected from 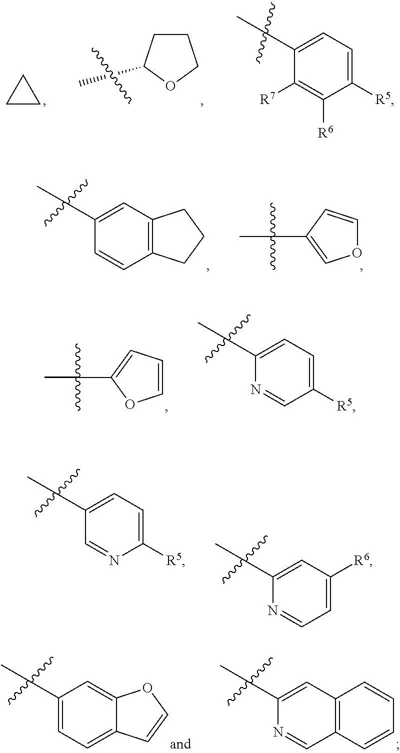
or a stereoisomer, a tautomer, a hydrate, a solvate, or a salt thereof, or a mixture of same.

2. The compound according to claim 1, wherein
R¹ is selected from a hydrogen atom, a halogen atom, a C₁-C₄-alkyl group, a C₁-C₄-haloalkyl group, a C₃-C₆-cycloalkyl group, a C₁-C₄-alkoxy group and a phenyl group which is optionally substituted with a halogen atom, a C₁-C₃-alkoxy group, a C₁-C₃-alkyl group;
R² is selected from a hydrogen atom, a C₁-C₄-alkyl group, a —O—(CH₂)₂—NR⁸R⁹ group and a cyano group;
R³ is selected from a hydrogen atom, a cyano group, a NR⁸R⁹ group, a C₁-C₄-alkyl group, a C₁-C₄-hydroxyalkyl group and a heterocycloalkyl group;
R⁴ is hydrogen;
R⁵ is selected from a hydrogen atom, a halogen atom, a cyano group, a C₁-C₄-alkyl group and a N(O)₂ group;
R⁶ is selected from a hydrogen atom, a halogen atom, a C₁-C₄-alkyl group, a C₂-C₄-alkenyl group and a C₂-C₄-alkynyl group;
R⁷ is selected from a hydrogen atom, a halogen atom and a C₁-C₄-alkyl group;
R⁸, R⁹ is independently selected from a hydrogen atom and a C₁-C₄-alkyl group;
A is selected from 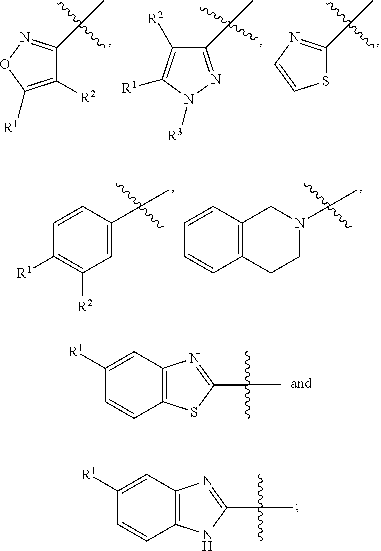
L¹ is selected from ^{$}—NH—C(O)—^{$$}, ^{$}—(CH₂)₃—NH—C(O)—^{$$}, ^{$}—(CH₂)₄—NH—C(O)—^{$$}, 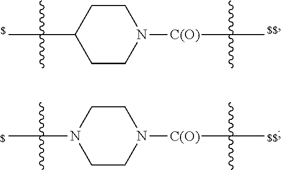
^{$} is the point of attachment of L¹ to ring A and ^{$$} is the point of attachment of L¹ to the thiazole carbon atom;
L² is selected from a bond, *—NH—**, *—NH—CH₂—**, *—NH—(CH₂)₂—**, 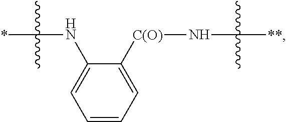
*—CH₂—**; and * is the point of attachment of L² to the thiazole carbon atom and ** is the point of attachment of L² to ring B;
B is selected from 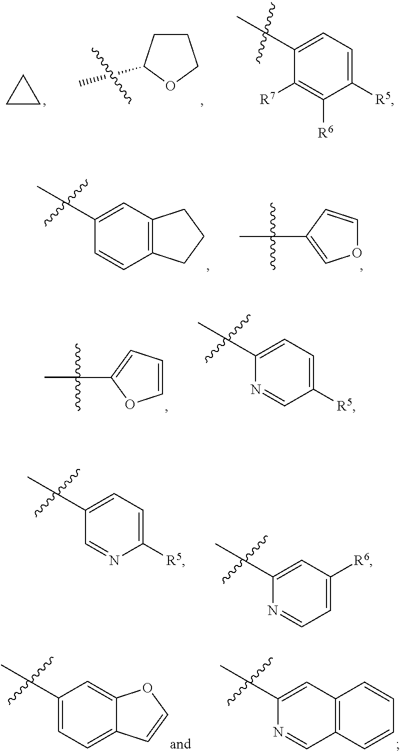
or a stereoisomer, a tautomer, a hydrate, a solvate, or a salt thereof, or a mixture of same.

3. The compound according to claim 1, wherein:
R¹ is selected from a hydrogen atom, a C₁-C₄-alkyl group, a C₁-C₃-haloalkyl group, a C₃-C₆-cycloalkyl group, a C₁-C₃-alkoxy group and a phenyl group which is optionally substituted with a halogen atom, a C₁-C₃-alkoxy group, a C₁-C₃-alkyl group;
R² is selected from a hydrogen atom, a C₁-C₃-alkyl group, a —O—(CH₂)₂—NR⁸R⁹ group and a cyano group;
R³ is selected from a hydrogen atom, a cyano group, a NR⁸R⁹ group, a C₁-C₃-alkyl group, a C₁-C₃-hydroxyalkyl group and a heterocycloalkyl group;
R⁴ is hydrogen;
R⁵ is selected from a hydrogen atom, a fluorine atom, a chlorine atom, a cyano group, a C₁-C₃-alkyl group and a N(O)₂ group;
R⁶ is selected from a hydrogen atom, a fluorine atom, a chlorine atom, a C₁-C₃-alkyl group and a C₂-C₃-alkynyl group;
R⁷ is selected from a hydrogen atom, a fluorine atom and a C₁-C₃-alkyl group;
R⁸, R⁹ is independently selected from a hydrogen atom and a C₁-C₃-alkyl group;
A is selected from 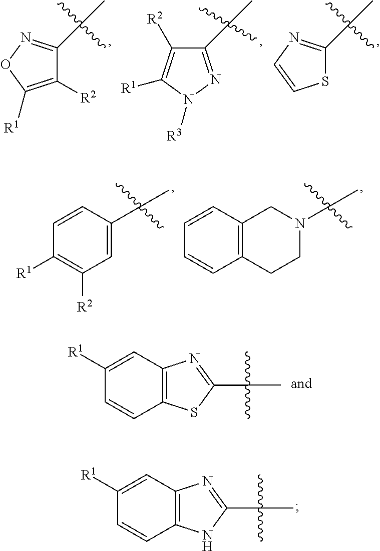
L¹ is selected from ^{$}—NH—C(O)—^{$$}, ^{$}—(CH₂)₃—NH—C(O)—^{$$}, ^{$}—(CH₂)₄—NH—C(O)—^{$$}, 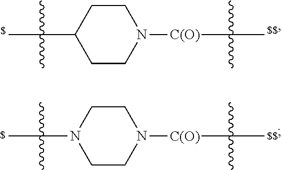
and ^{$} is the point of attachment of L¹ to ring A and ^{$$} is the point of attachment of L¹ to the thiazole carbon atom;
L² is selected from a bond, *—NH—**, *—NH—CH₂—**, *—NH—(CH₂)₂—**, 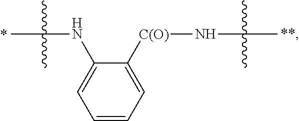
*—CH₂—**; and * is the point of attachment of L² to the thiazole carbon atom and ** is the point of attachment of L² to ring B;
B is selected from 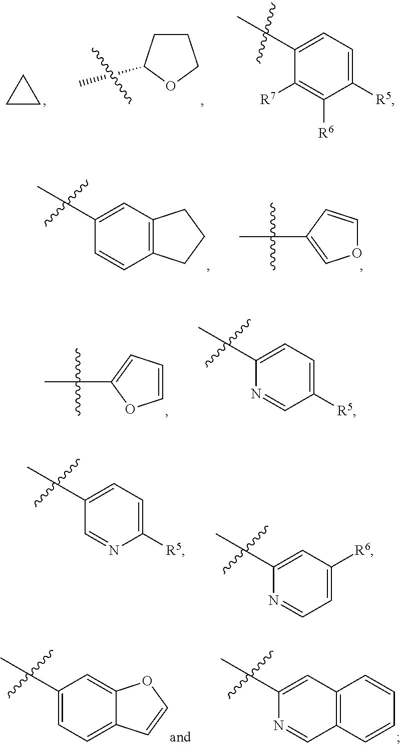
or a stereoisomer, a tautomer, a hydrate, a solvate, or a salt thereof, or a mixture of same.

4. The compound according to claim 1, wherein:
R¹ is selected from a hydrogen atom, a C₁-C₄-alkyl group, a C₁-C₃-haloalkyl group, a C₃-C₆-cycloalkyl group, a C₁-C₄-alkoxy group and a phenyl group which is optionally substituted with a C₁-C₄-alkyl group;
R² is selected from a hydrogen atom, a C₁-C₃-alkyl group, a —O—(CH₂)₂—NR⁸R⁹ group and a cyano group;
R³ is selected from a hydrogen atom, a cyano group, a C₁-C₃-alkyl group, a heterocycloalkyl group and a C₁-C₃-hydroxyalkyl group;
R⁴ is hydrogen;
R⁵ is selected from a hydrogen atom, a fluorine atom, a chlorine atom, a cyano group, a C₁-C₃-alkyl group, and a N(O)₂ group,
R⁶ is selected from a hydrogen atom, a fluorine atom, a chlorine atom, a C₁-C₃-alkyl group, and a C₂-C₄-alkynyl group,
R⁷ is selected from a hydrogen atom, a fluorine atom, and a C₁-C₃-alkyl group,
R⁸, R⁹ is independently selected from a hydrogen atom and a C₁-C₃-alkyl group;
A is selected from 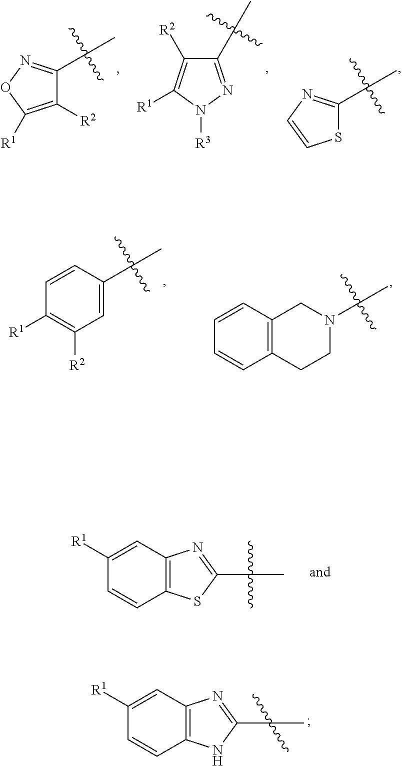
L¹ is selected from ^{$}—NH—C(O)—^{$$}, ^{$}—(CH₂)₃—NH—C(O)—^{$$}, ^{$}—(CH₂)₄—NH—C(O)—^{$$}, 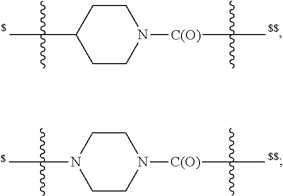
and ^{$} is the point of attachment of L¹ to ring A and ^{$$} is the point of attachment of L¹ to the thiazole carbon atom;
L² is selected from a bond, *—NH—**, *—NH—CH₂—**, *—NH—(CH₂)₂—**, 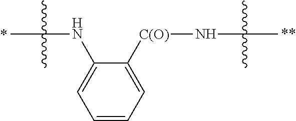
, *—CH₂—**; and * is the point of attachment of L² to the thiazole carbon atom and ** is the point of attachment of L² to ring B;
B is selected from 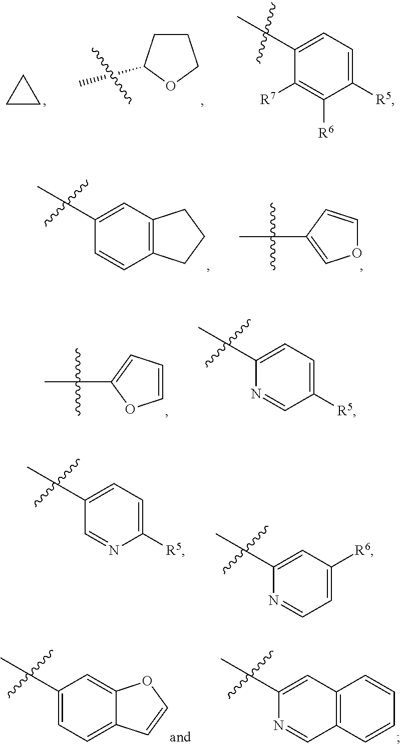
or a stereoisomer, a tautomer, a hydrate, a solvate, or a salt thereof, or a mixture of same.

5. The compound according to claim 1, wherein:
R¹ is selected from a hydrogen atom, a methyl group, a tert-butyl group, a trifluoromethyl group, a cyclopropyl group, a methoxy group, a phenyl group and a 4-methylphenyl group;
R² is selected from a hydrogen atom, a methyl group, a —O—(CH₂)₂—N(CH₂—CH₃)₂ group and a cyano group;
R³ is selected from a hydrogen atom, a cyano group, a methyl group, a oxetan-3-yl-group and a hydroxyethyl group;
R⁴ is hydrogen;
R⁵ is selected from a hydrogen atom, a fluorine atom, a chlorine atom, a cyano group, a methyl group and a N(O)₂ group,
R⁶ is selected from a hydrogen atom, a fluorine atom, a chlorine atom, a methyl group and a ethinyl group,
R⁷ is selected from a hydrogen atom, a fluorine atom, and a methyl group,
R⁸, R⁹ is independently selected from a hydrogen atom and a methyl group;
A is selected from 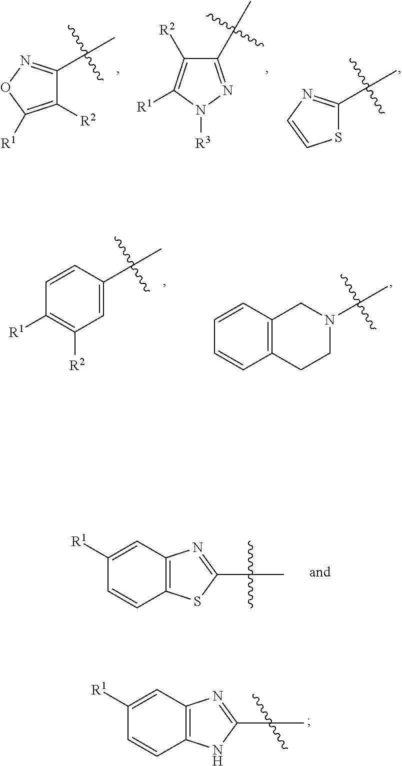
L¹ is selected from ^{$}—NH—C(O)—^{$$}, ^{$}—(CH₂)₃—NH—C(O)—^{$$}, ^{$}—(CH₂)₄—NH—C(O)—^{$$}, 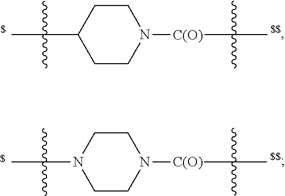
and ^{$} is the point of attachment of L¹ to ring A and $^{$$} is the point of attachment of L¹ to the thiazole carbon atom;
L² is selected from a bond, *—NH—**, *—NH—CH₂—**, *—NH—(CH₂)₂—**, 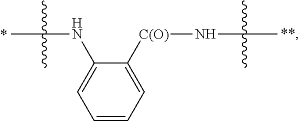
*—CH₂—**; and * is the point of attachment of L² to the thiazole carbon atom and ** is the point of attachment of L² to ring B;
B is selected from 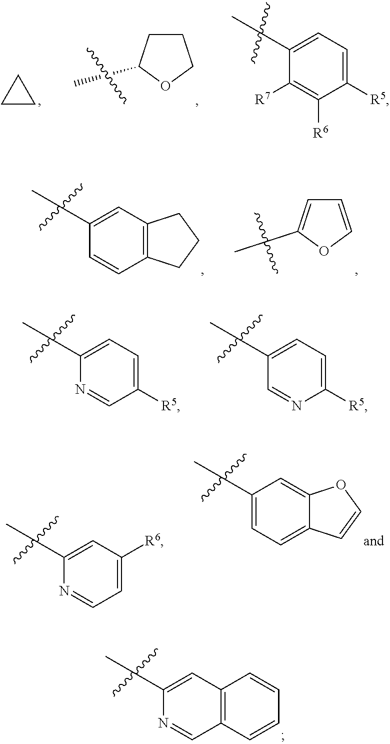
or a stereoisomer, a tautomer, a hydrate, a solvate, or a salt thereof, or a mixture of same.

6. The compound according to claim 1 which is selected from the group consisting of:
2-[(4-Chlorophenyl)amino]-N-(5-phenyl-1H-pyrazol-3-yl)-1,3-thiazole-4-carboxamide,
N-(5-tert-Butyl-1,2-oxazol-3-yl)-2-(pyridin-2-ylamino)-1,3-thiazole-4-carboxamide,
N-(5-tert-Butyl-1,2-oxazol-3-yl)-2-(phenylamino)-1,3-thiazole-4-carboxamide,
N-(5-tert-Butyl-1,2-oxazol-3-yl)-2-[(4-chlorophenyl)amino]-1,3-thiazole-4-carboxamide,
2-[(4-Chlorophenyl)amino]-N-(3-cyclopropyl-1H-pyrazol-5-yl)-1,3-thiazole-4-carboxamide,
N-(5-tert-Butyl-1,2-oxazol-3-yl)-2-[(5-fluoropyridin-2-yl)amino]-1,3-thiazole-4-carboxamide,
N-(5-tert-Butyl-1,2-oxazol-3-yl)-2-[(4-fluorophenyl)amino]-1,3-thiazole-4-carboxamide,
N-(1,3-Benzothiazol-2-yl)-2-[(4-chlorophenyl)amino]-1,3-thiazole-4-carboxamide,
2-[(4-Chlorophenyl)amino]-N-(5-methoxy-1H-benzimidazol-2-yl)-1,3-thiazole-4-carboxamide,
N-[1-Methyl-5-(trifluoromethyl)-1H-pyrazol-3-yl]-2-(phenylamino)-1,3-thiazole-4-carboxamide,
2-[(4-Chlorophenyl)amino]-N-(1,3-thiazol-2-yl)-1,3-thiazole-4-carboxamide,
N-(5-tert-Butyl-1H-pyrazol-3-yl)-2-[(4-chlorophenyl)amino]-1,3-thiazole-4-carboxamide,
N-(5-tert-Butyl-1,2-oxazol-3-yl)-2-[(4-nitrophenyl)amino]-1,3-thiazole-4-carboxamide,
N-(3-Cyclopropyl-1H-pyrazol-5-yl)-2-(phenylamino)-1,3-thiazole-4-carboxamide,
2-[(4-Chlorophenyl)amino]-N-(1-ethyl-1H-pyrazol-3-yl)-1,3-thiazole-4-carboxamide,
N-(5-tert-Butyl-1H-pyrazol-3-yl)-2-(phenylamino)-1,3-thiazole-4-carboxamide,
2-[(4-Chlorophenyl)amino]-N-[4-(3,4-dihydroisoquinolin-2(1H)-yl)butyl]-1,3-thiazole-4-carboxamide,
N-(5-tert-Butyl-1,2-oxazol-3-yl)-2-[(3-chloro-4-methylphenyl)amino]-1,3-thiazole-4-carboxamide,
2-[(4-Chlorophenyl)amino]-N-(1,2-oxazol-3-yl)-1,3-thiazole-4-carboxamide,
2-[(4-Chlorophenyl)amino]-N-(5-methyl-1,2-oxazol-3-yl)-1,3-thiazole-4-carboxamide,
N-(5-tert-Butyl-1,2-oxazol-3-yl)-2-[(3,4-dichlorophenyl)amino]-1,3-thiazole-4-carboxamide,
N-{2-[2-(Diethylamino)ethoxy]-4′-methylbiphenyl-4-yl}-2-(phenylamino)-1,3-thiazole-4-carboxamide,
N-(1-Methyl-1H-pyrazol-3-yl)-2-(phenylamino)-1,3-thiazole-4-carboxamide,
N-(5-tert-Butyl-1,2-oxazol-3-yl)-2-[(4-cyanophenyl)amino]-1,3-thiazole-4-carboxamide,
N-(5-tert-Butyl-1,2-oxazol-3-yl)-2-[(2-fluorophenyl)amino]-1,3-thiazole-4-carboxamide,
2-[(4-Chlorophenyl)amino]-N-[3-(3,4-dihydroisoquinolin-2(1H)-yl)propyl]-1,3-thiazole-4-carboxamide,
{2-[(4-Chlorophenyl)amino]-1,3-thiazol-4-yl}{4-[3,4-dihydroisoquinolin-2(1H)-yl]piperidin-1-yl}methanone,
N-[1-(2-Hydroxyethyl)-1H-pyrazol-3-yl]-2-(phenylamino)-1,3-thiazole-4-carboxamide,
N-(5-tert-Butyl-4-methyl-1,2-oxazol-3-yl)-2-[(4-chlorophenyl)amino]-1,3-thiazole-4-carboxamide,
N-(4-Cyano-1H-pyrazol-3-yl)-2-(phenylamino)-1,3-thiazole-4-carboxamide,
2-[(4-Chlorophenyl)amino]-N-[1-(oxetan-3-yl)-1H-pyrazol-4-yl]-1,3-thiazole-4-carboxamide,
N-(5-tert-Butyl-1,2-oxazol-3-yl)-2-(cyclopropylamino)-1,3-thiazole-4-carboxamide, and
{2-[(4-Chlorophenyl)amino]-1,3-thiazol-4-yl}[4-(5-methyl-1,3-benzothiazol-2-yl)piperazin-1-yl]methanone
N-(5-tert-Butyl-1,2-oxazol-3-yl)-2-[(5-methylpyridin-2-yl)amino]-1,3-thiazole-4-carboxamide,
N-(5-tert-Butyl-1,2-oxazol-3-yl)-2-[(5-chloropyridin-2-yl)amino]-1,3-thiazole-4-carboxamide,
N-(5-tert-Butyl-1,2-oxazol-3-yl)-2-[(4-chloropyridin-2-yl)amino]-1,3-thiazole-4-carboxamide,
N-(5-tert-Butyl-1,2-oxazol-3-yl)-2-[(2-phenylethyl)amino]-1,3-thiazole-4-carboxamide,
N-(5-tert-Butyl-1,2-oxazol-3-yl)-2-(2,3-dihydro-1H-inden-5-ylamino)-1,3-thiazole-4-carboxamide,
N-(5-tert-Butyl-1,2-oxazol-3-yl)-2-[(4-chloro-3-fluorophenyl)amino]-1,3-thiazole-4-carboxamide,
2-(1-Benzofuran-6-ylamino)-N-(5-tert-butyl-1,2-oxazol-3-yl)-1,3-thiazole-4-carboxamide,
N-(5-tert-Butyl-1,2-oxazol-3-yl)-2-[(2-fluoro-4-methylphenyl)amino]-1,3-thiazole-4-carboxamide,
N-(5-tert-Butyl-1,2-oxazol-3-yl)-2-[(3-fluorobenzyl)amino]-1,3-thiazole-4-carboxamide,
N-(5-tert-Butyl-1,2-oxazol-3-yl)-2-[(3-methylphenyl)amino]-1,3-thiazole-4-carboxamide,
N-(5-tert-Butyl-1,2-oxazol-3-yl)-2-[(furan-2-ylmethyl)amino]-1,3-thiazole-4-carboxamide,
N-(5-tert-Butyl-1,2-oxazol-3-yl)-2-[(3,4-difluorophenyl)amino]-1,3-thiazole-4-carboxamide,
N-(5-tert-Butyl-1,2-oxazol-3-yl)-2-[(3-chloro-4-fluorophenyl)amino]-1,3-thiazole-4-carboxamide,
N-(5-tert-Butyl-1,2-oxazol-3-yl)-2-{[2-(isoquinolin-3-ylcarbamoyl)phenyl]amine}-1,3-thiazole-4-carboxamide,
N-(5-tert-Butyl-1,2-oxazol-3-yl)-2-[(3-chlorophenyl)amino]-1,3-thiazole-4-carboxamide,
(rac)-N-(5-tert-Butyl-1,2-oxazol-3-yl)-2-{[-tetrahydrofuran-2-ylmethyl]amine}-1,3-thiazole-4-carboxamide,
N-(5-tert-Butyl-1,2-oxazol-3-yl)-2-[(4-fluoro-2-methylphenyl)amino]-1,3-thiazole-4-carboxamide,
N-(5-tert-Butyl-1,2-oxazol-3-yl)-2-[(3-ethynylphenyl)amino]-1,3-thiazole-4-carboxamide,
N-(5-tert-Butyl-1,2-oxazol-3-yl)-2-[(2,3-difluorophenyl)amino]-1,3-thiazole-4-carboxamide and
N-(5-tert-Butyl-1,2-oxazol-3-yl)-2-[(6-fluoropyridin-3-yl)amino]-1,3-thiazole-4-carboxamide
or a stereoisomer, a tautomer, a hydrate, a solvate, or a salt thereof, or a mixture of same.

7. A method of preparing a compound according to claim 1, said method comprising the step of reacting an intermediate compound of general formula (II): 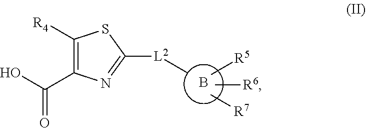
in which R⁴, R⁵, R⁶, R⁷ and L² are as defined in claim 1,
with a compound of general formula (III): 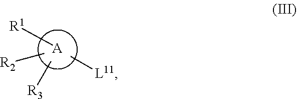
in which R¹, R², R³ are as defined in claim 1, and 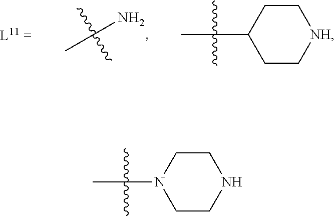
thereby giving a compound of general formula (I): 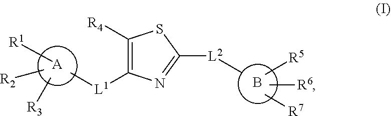
in which R¹, R², R³ R⁴, R⁵, R⁶, R⁷ L¹ and L² are as defined in claim 1, then optionally removing any protecting groups used and optionally converting said compound into solvates, salts and/or solvates of such salts using the corresponding (i) solvents and/or (ii) bases or acids.

8. A method of preparing a compound according to claim 1, said method comprising the step of reacting an intermediate compound of general formula (IV): 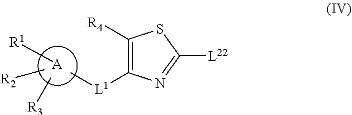
in which R¹, R², R³, R⁴ and L¹ are as defined in claim 1 and,
L²² is a halogenide,
with a compound of general formula (V): 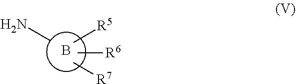
in which R⁵, R⁶, R⁷ and R⁴ are as defined in claim 1, in the presence of a base alone or additionally in the presence of a palladium or copper catalyst,
thereby giving a compound of general formula (I): 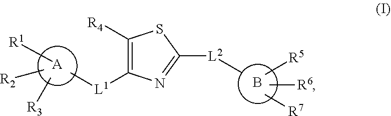
in which R¹, R², R³ R⁴, R⁵, R⁶, R⁷ L¹ and L² are as defined in claim 1,
then optionally removing any protecting groups used and optionally converting said compound into solvates, salts and/or solvates of such salts using the corresponding (i) solvents and/or (ii) bases or acids.

9. A pharmaceutical composition comprising a compound according to claim 1 and one or more pharmaceutically acceptable excipients.

10. A pharmaceutical combination comprising:
one or more first active ingredients, selected from compounds according to claim 1, and
one or more further active ingredients.

11. The pharmaceutical combination according to claim 10, wherein the one or more further active ingredients are selected from anti-cancer agents.

12. (canceled)

13. A method for the treatment or prophylaxis of a hyperproliferative disorder comprising administering to a subject in need thereof a therapeutically or prophylactically effective amount of a compound according to claim 1.

14. The method according to claim 13, wherein the hyperproliferative disorder is cancer.

15. A compound of general formula (II) or (IV): 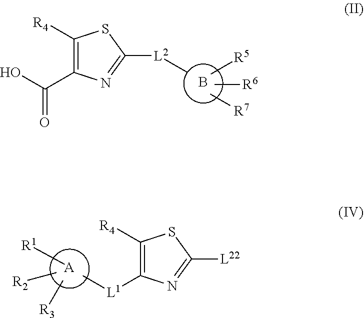
in which R¹, R², R³, R⁴, R⁵, R⁶, R⁷ and L¹ and L² are as defined in claim 1 and,
L²² is a halogenide,

16. (canceled)
